(19) United States
(12) Patent Application Publication
(10) Pub. No.: US 2022/0002396 A1
(43) Pub. Date: Jan. 6, 2022

(54) METHODS OF TREATING CANCER WITH FARNESYLTRANSFERASE INHIBITORS

(71) Applicant: Kura Oncology, Inc., San Diego, CA (US)

(72) Inventor: Antonio Gualberto, Boston, MA (US)

(21) Appl. No.: 17/290,157

(22) PCT Filed: Oct. 31, 2019
(86) PCT No.: PCT/US2019/059084

§ 371 Date: Apr. 29, 2021

Related U.S. Application Data
(60) Provisional application No. 62/793,547, filed on Jan. 17, 2019.
(60) Provisional application No. 62/754,438, filed on Nov. 1, 2018.

(51) Int. Cl. C07K 16/22; A61K 31/7068; A61K 31/53; A61K 38/07; A61K 31/5377; A61K 31/437; A61K 31/52; A61K 31/427; A61K 31/498; A61K 31/4745; A61K 31/519; A61K 31/366; A61K 31/675; A61K 31/404; A61K 31/4418; A61K 31/7064; A61K 31/4545; A61K 31/352; A61K 39/395; A61K 31/395; A61K 38/12; A61K 31/4706; A61K 31/5513; A61K 31/4704; A61K 31/365; A61K 31/05; A61K 31/223; A61K 31/4406; A61K 31/496; A61P 35/00

(52) U.S. Cl. CPC C07K 16/22; A61P 35/00; A61K 31/53; A61K 38/07; A61K 31/5377; A61K 31/437; A61K 31/52; A61K 31/427; A61K 31/498; A61K 31/4745; A61K 31/519; A61K 31/366; A61K 31/675; A61K 31/404; A61K 31/4418; A61K 31/7064; A61K 31/4545; A61K 31/352; A61K 39/3955; A61K 31/395; A61K 38/12; A61K 31/4706; A61K 31/5513; A61K 31/4704; A61K 31/365; A61K 31/05; A61K 31/223; A61K 31/4406; A61K 31/496; A61K 31/7068

(57) ABSTRACT

The present invention relates to the field of cancer therapy. Specifically, provided are methods of treating cancer in a subject with a farnesyltransferase inhibitor (FTI) that include determining whether the subject is likely to be responsive to the FTI treatment based on the activity of the CXCL12/CXCR4 pathway, and/or the activity of the IGF1R pathway. Provided herein are also combination therapy of cancer treatment using FTI and either an IGF1R inhibitor or a CXCR4 antagonist.

This application claims benefit of priority of U.S. Provisional Application No. 62/754,438, filed on Nov. 01, 2018, and U.S. Provisional Application No. 62/793,547, filed on Jan. 17, 2019, the entire contents of which are incorporated herein by reference.

### FIELD

The present invention relates to the field of cancer therapy. In particular, provided herein are methods of treating cancers with farnesyltransferase inhibitors.

### BACKGROUND

Stratification of patient populations to improve therapeutic response rate is increasingly valuable in the clinical management of cancer patients. Farnesyltransferase inhibitors (FTI) are therapeutic agents that have utility in the treatment of cancers. However, patients respond differently to an FTI treatment. Therefore, methods to predict the responsiveness of a subject having cancer to an FTI treatment, or methods to select cancer patients for an FTI treatment, represent unmet needs. The methods and compositions provided herein meet these needs and provide other related advantages.

### SUMMARY

Provided herein is a method of treating a KRAS wild type cancer in a subject, comprising administering a therapeutically effective amount of a farnesyltransferase inhibitor (FTI) to the subject.

In some embodiments, the subject has a greater C-X-C motif chemokine ligand 12 (CXCL12) expression than a reference level of CXCL12 expression.

In some embodiments, the cancer is a solid tumor.

In some embodiments, the solid tumor is pancreatic cancer.

In some embodiments, the cancer is pancreatic ductal adenocarcinoma (PDAC).

In some embodiments, the tumor has a KRAS Variant Allele Frequency (VAF) of less than or equal to 5%.

In some embodiments, the KRAS status is assessed at primary diagnosis or in recurrent or metastatic disease.

Provided herein is a method of treating a cancer in a subject comprising administering a therapeutically effective amount of a farnesyltransferase inhibitor (FTI) to the subject, wherein the subject has (i) (a) a greater C-X-C motif chemokine ligand 12 (CXCL12) expression than a reference level of CXCL12 expression; or (b) a CXCR4 expression greater than a reference level of CXCR4 expression; and (ii) (a) a lower insulin-like growth factor 1 (IGF1) expression than a reference level of IGF1 expression; or (b) a greater insulin-like growth factor binding protein 7 (IGFBP7) expression than a reference level of IGFBP7 expression.

In some embodiments, the subject has an IGF1 expression that is non-detectable.

In some embodiments, the subject further has (i) a lower insulin-like growth factor 2 (IGF2) expression than a reference level of IGF2 expression, or (ii) a greater insulin-like growth factor 2 receptor (IGF2R) expression than a reference level of IGF2R expression. In some embodiments, the subject has an IGF2 expression that is non-detectable. In some embodiments, the subject does not have a loss of heterozygosity or loss of imprinting of the IGF2 gene.

In some embodiments, the subject does not carry the IGFBP7 variant L11F (rs11573021).

In some embodiments, the subject has a greater ratio of expression of CXCL12 to C-X-C chemokine receptor type 4 (CXCR4) than a reference ratio.

In some embodiments, the subject further has an activating mutation in the CXCR4 gene.

In some embodiments, the subject further has a greater ratio of CXCR4 to CXCR2 expression than a reference ratio.

In some embodiments, the subject has a KRAS mutation allele frequency that is less than 15%, less than 12%, less than 10%, less than 8%, less than 7%, less than 6%, or less than 5%. In some embodiments, the subject does not have an activating mutation in the KRAS gene.

In some embodiments, the subject has a TP53 mutation allele frequency that is less than 15%, less than 12%, less than 10%, less than 8%, less than 7%, less than 6%, or less than 5%. In some embodiments, the subject does not have a mutation in the TP53 gene.

In some embodiments, the subject does not have an activating mutation in PI3K or AKT.

In some embodiments, the methods provided herein further comprise measuring the expression level of CXCL12, IGF1, IGFBP7, IGF2, IGF2R, CXCR4, CXCR2, or any combination thereof, in a sample of the subject.

In some embodiments, the methods provided herein further comprise measuring the protein level of CXCL12, IGF1, IGFBP7, IGF2, IGF2R, CXCR4, CXCR2, or any combination thereof, in the sample.

In some embodiments, the protein level is determined using a immunohistochemistry (IHC) approach, an immunoblotting assay, flow cytometry (FACS), or ELISA.

In some embodiments, the methods provided herein further comprise measuring the mRNA level of CXCL12, IGF1, IGFBP7, IGF2, IGF2R, CXCR4, CXCR2, or any combination thereof, in the sample.

In some embodiments, the mRNA level is measured using qPCR, RT-PCR, RNA-seq, microarray analysis, SAGE, MassARRAY technique, or FISH.

In some embodiments, the methods provided herein further comprise determining the mutation status of IGFBP7, KRAS, TP53, PI3K, AKT, CXCR4 or any combination thereof.

In some embodiments, the sample is a tissue biopsy. In some embodiments, the sample is a tumor biopsy. In some embodiments, the sample is isolated cells.

In some embodiments, the cancer is a hematological cancer. In some embodiments, the hematological cancer is selected from the group consisting of acute myeloid leukemia (AML), myeloproliferative neoplasm (MPN), myelodysplastic syndrome (MDS), chronic myelomonocytic leukemia (CMML) and chronic myeloid leukemia (CML). In some embodiments, the hematological cancer is AML.

In some embodiments, the hematological cancer is a lymphoid hematological cancer selected from the group consisting of natural killer cell lymphoma (NK lymphoma), natural killer cell leukemia (NK leukemia), cutaneous T-Cell lymphoma (CTCL), and peripheral T-cell lymphoma (PTCL). In some embodiments, the hematological cancer is PTCL.

In some embodiments, the cancer is a solid tumor selected from the group consisting of pancreatic cancer, bladder cancer, breast cancer, gastric cancer colorectal cancer, head and neck cancer, mesothelioma, uveal melanoma, glioblastoma, adrenocortical carcinoma, esophageal cancer, melanoma, lung adenocarcinoma, prostate cancer, lung squamous carcinoma, ovarian cancer, hepatocellular carcinoma, sarcoma, and prostate cancer. In some embodiments, the solid tumor is pancreatic cancer, bladder cancer, breast cancer or gastric cancer.

In some embodiments, the solid tumor is breast cancer. In some embodiments, the breast cancer is progesterone receptor (PR) positive. In some embodiments, the breast cancer is estrogen receptor (ER) negative.

In some embodiments, provided herein are methods of treating a pancreatic cancer in a subject comprising administering a therapeutically effective amount of a FTI to said subject, wherein the subject has (i) liver metastases; and (ii) (1) an aspartate transaminase (AST) level, (2) an alanine transaminase level, (3) an alkaline phosphatase, and/or (4) a total bilirubin level that is no more than the normal upper limit.

In some embodiments, provided herein are methods of treating a pancreatic cancer in a subject comprising administering a therapeutically effective amount of a FTI to said subject, wherein the subject (i) has nodal metastasis, and (ii) does not have abdominal pain.

In some embodiments, the solid tumor is pancreatic ductal adenocarcinoma (PDAC).

In some embodiments, the methods provided herein further comprise administering an inhibitor of IGF1R pathway to said subject. In some embodiments, the FTI is administered before, during, or after the administration of the inhibitor of IGF1R pathway.

In some embodiments, the inhibitor of IGF1R pathway is selected from the group consisting of an IGF1 inhibitor, an IGF1R inhibitor, a PI3K inhibitor, and an AKT inhibitor.

In some embodiments, the inhibitor of IGF1R pathway is an anti-IGF1 antibody. In some embodiments, the inhibitor of IGF1R pathway can be an IGF1R inhibitor. In some embodiments, the IGF1R inhibitor is selected from the group consisting of dalotuzumab, robatumumab, figitumumab, cixutumumab, ganitumab, AVE1642, OSI-906, NVP-AEW541 and NVP-ADW742.

In some embodiments, the inhibitor of IGF1R pathway is a PI3K inhibitor. In some embodiments, the PI3K inhibitor is selected from the group consisting of SF1126, TGX-221, PIK-75, PI-103, SN36093, IC87114, AS-252424, AS-605240, NVP-BEZ235, GDC-0941, ZSTK474, LY294002 and wortmannin.

In some embodiments, the inhibitor of IGF1R pathway is an AKT inhibitor. In some embodiments, the AKT inhibitor is selected from the group consisting of perifosine, SR13668, A-443654, triciribine phosphate monohydrate, GSK690693, and deguelin.

In some embodiments, the methods provided herein further comprise administering a radiation therapy.

In some embodiments, the methods provided herein further comprise administering a therapeutically effective amount of an additional active agent.

In some embodiments, the additional active agent is selected from the group consisting of a DNA-hypomethylating agent, an alkylating agent, a topoisomerase inhibitor, a therapeutic antibody that specifically binds to a cancer antigen, a hematopoietic growth factor, a cytokine, an antibiotic, a cox-2 inhibitor, a CDK inhibitor, an immunomodulatory agent, an anti-thymocyte globulin, an immunosuppressive agent, and a corticosteroid or a pharmacological derivative thereof.

In some embodiments, said additional active agent is capecitabine. In some embodiments, the capecitabine is administered at a dose of 1-1000 mg/m². In some embodiments, the capecitabine is administered twice a day. In some embodiments, the capecitabine is administered on days 1-7 of 21-day cycles. In some embodiments, the capecitabine is administered on days 1-14 of 21-day cycle.

In some embodiments, the additional active agent is an anti-PD1 antibody, an anti-PDL1 antibody, or an anti-CTLA-4 antibody.

In some embodiments, provided herein are methods of treating a cancer in a subject, comprising administering a therapeutically effective amount of a FTI and a therapeutically effective amount of (i) an inhibitor of IGF1R pathway or (ii) an CXCR4 antagonist to said subject.

In some embodiments, the cancer is a solid tumor. In some embodiments, the solid tumor is selected from the group consisting of pancreatic cancer, bladder cancer, breast cancer, gastric cancer colorectal cancer, head and neck cancer, mesothelioma, uveal melanoma, glioblastoma, adrenocortical carcinoma, esophageal cancer, melanoma, lung adenocarcinoma, prostate cancer, lung squamous carcinoma, ovarian cancer, hepatocellular carcinoma, sarcoma, and prostate cancer.

In some embodiments, the solid tumor is pancreatic cancer, bladder cancer, breast cancer or gastric cancer. In some embodiments, the solid tumor is pancreatic cancer. In some embodiments, the pancreatic cancer is pancreatic ductal adenocarcinoma (PDAC). In some embodiments, the solid tumor is breast cancer.

In some embodiments, the cancer is a hematological cancer. In some embodiments, the hematological cancer is selected from the group consisting of acute myeloid leukemia (AML), myeloproliferative neoplasm (MPN), myelodysplastic syndrome (MDS), chronic myelomonocytic leukemia (CMML), chronic myeloid leukemia (CML), natural killer cell lymphoma (NK lymphoma), natural killer cell leukemia (NK leukemia), cutaneous T-Cell lymphoma (CTCL), and peripheral T-cell lymphoma (PTCL).

In some embodiments, the FTI is administered before, during, or after the administration of the inhibitor of IGF1R pathway or the CXCR4 antagonist.

In some embodiments, methods provided herein comprise administering an FTI with an CXCR4 antagonist that is selected from the group consisting of AMD-3100, BL-8040, chloroquine, and plerixafor.

In some embodiments, methods provided herein further comprise administering an FTI with an inhibitor of IGF1R pathway that is selected from the group consisting of an IGF1 inhibitor, an IGF1R inhibitor, a PI3K inhibitor, and an AKT inhibitor.

In some embodiments, the inhibitor of IGF1R pathway is an anti-IGF1 antibody. In some embodiments, the inhibitor of IGF1R pathway is an IGF1R inhibitor. In some embodiments, the IGF1R inhibitor is selected from the group consisting of dalotuzumab, robatumumab, figitumumab, cixutumumab, ganitumab, AVE1642, OSI-906, NVP-AEW541 and NVP-ADW742.

In some embodiments, the inhibitor of IGF1R pathway is a PI3K inhibitor. In some embodiments, the PI3K is inhibitor selected from the group consisting of SF1126, TGX-221, PIK-75, PI-103, SN36093, IC87114, AS-252424, AS-605240, NVP-BEZ235, GDC-0941, ZSTK474, LY294002 and wortmannin.

In some embodiments, the inhibitor of IGF1R pathway is an AKT inhibitor. In some embodiments, the AKT inhibitor is selected from the group consisting of perifosine, SR13668, A-443654, triciribine phosphate monohydrate, GSK690693, and deguelin.

In some embodiments, the FTI used in the methods provided herein is selected from the group consisting of tipifarnib, lonafarnib, arglabin, perrilyl alcohol, L778123, L739749, FTI-277, L744832, CP-609,754, R208176, AZD3409, and BMS-214662.

In some embodiments, the FTI is lonafarnib. In some embodiments, the FTI is BMS-214662.

In some embodiments, the FTI is tipifarnib. In some embodiments, the tipifarnib is administered at a dose of 0.05-500 mg/kg body weight.

In some embodiments, the tipifarnib is administered twice a day. In some embodiments, the tipifarnib is administered at a dose of 100-1200 mg twice a day. In some embodiments, the tipifarnib is administered at a dose of 100 mg, 200 mg, 300 mg, 400 mg, 600 mg, 900 mg or 1200 mg twice a day.

In some embodiments, the tipifarnib is administered on days 1-7 and 15-21 of a 28-day treatment cycle. In some embodiments, the tipifarnib is administered on days 1-21 of a 28-day treatment cycle. In some embodiments, the tipifarnib is administered on days 1-7 of a 28-day treatment cycle.

In some embodiments, the tipifarnib is administered on days 1-7 of 21-day cycles. In some embodiments, the tipifarnib is administered on days 1-14 of 21-day cycles. In some embodiments, the tipifarnib is administered at a dose of 300 mg twice daily on days 1-14 of 21-day cycles and the capecitabine is administered at a dose of 1,000 mg/m2 twice daily on days 1-14 of 21-day cycles.

In some embodiments, the tipifarnib is administered for at least 2 cycles. In some embodiments, the tipifarnib is administered for at least 3 cycles, 6 cycles, 9 cycles, or 12 cycles. In some embodiments, the therapy can be maintained for at least 6 months beyond the start of the response.

### BRIEF DESCRIPTION OF THE DRAWINGS

The foregoing and other objects, features and advantages will be apparent from the following description of particular embodiments of the invention, as illustrated in the accompanying drawings. The drawings are not necessarily to scale, emphasis instead being placed upon illustrating the principles of various embodiments of the invention.

FIG. 1. Correlation of the expression of the CXCL12 and IGF1 genes in breast cancer, pancreatic cancer and acute myeloid leukemia.

FIGS. 2A-2E. The effect of IGF1/CXCL12 co-expression on patients' responsiveness to tipifarnib treatment in patients with previously untreated AML. FIG. 2A: Three subsets of AML patients were identified with respect to IGF1/CXCL12 expression. FIG. 2B: Subset of patients with high IGF1 and high CXCL12. FIG. 2C: Subset of patients with intermediate IGF1, low CXCL12. FIG. 2D: Subset of patients with low IGF1 and variable CXCL12. FIG. 2E: ROC curves for the prediction of a complete response based on pre-treatment CXCL12/IGF1 expression in bone marrow of AML patients.

FIG. 3: Pancreatic cancer expresses the highest levels of IGFBP (TCGA, Provisional).

FIG. 4: Schema of subsets of pancreatic tumors that are likely to respond to tipifarnib treatment based on CXCL12/CXCR4 and IGF1/IGF1R-CXCR4 mechanisms.

FIGS. 5A-5C. Kaplan Meier curves generated from study INT-11 of gemcitabine+tipifarnib versus gemcitabine+placebo in patients with local or nodal pancreatic adenocarcinoma and in those who do not report abdominal pain. FIG. 5A: Kaplan Meier curves of survival probability for all patients in the study (left), for those with locally advanced disease (middle), and for those with nodal metastases (right). FIG. 5B: Kaplan Meier curves of survival probability according to patient reported abdominal pain for patients receiving placebo+gemcitabine (left) or tipifarnib+gemcitabine (right). FIG. 5C: Kaplan Meier curves of survival probability for patients in the study who reported no abdominal pain.

FIGS. 6A-6C: Kaplan Meier curves generated from study INT-11 of gemcitabine+tipifarnib versus gemcitabine+placebo in patients with advanced pancreatic adenocarcinoma that metastasize to the liver. FIG. 6A: Kaplan Meier curves generated from study INT-11 of gemcitabine+tipifarnib versus gemcitabine+placebo for patients with metastases only to the liver and no elevated aspartate transaminase. FIG. 6B: Kaplan Meier curves generated from study INT-11 of gemcitabine+tipifarnib versus gemcitabine+placebo for patients with metastases only to the liver and had been previously treated with folfirinox. FIG. 6C: Kaplan Meier curves generated from study INT-11 of gemcitabine+tipifarnib versus gemcitabine+placebo for patients with liver metastases and no hyperglycemia (left), and for patients with liver metastases, no elevated aspartate transaminase and no hyperglycemia (right).

FIGS. 7A-7B: Low allele frequency of KRAS or TP53 mutation identifies pancreatic patients with high CXCL12, IGF1 and IGFBP1 expression. FIG. 7A: CXCL12, IGF1 and IGFBP7 gene expression levels in pancreatic adenocarcinoma of TCGA study according to KRAS mutation subsets. FIG. 7B: Optimal cut-off of TP53 mutation allele frequency for CXCL12 overexpression was <9% (left) and optimal cut-off of KRAS mutation allele frequency for CXCL12 overexpression was <7% (right).

FIG. 8: Administration of tipifarnib treatment regimen (tipifarnib alone or tipifarnib-atorvastatin-celecoxib combination) reduces tumor volume in PDX models of KRAS wild type (WT), CXCL12^{high} expressing-PDAC.

FIG. 9: Administration of tipifarnib was more effective at reducing tumor volume in KRAS wild type (WT) tumors than tumors with G12D KRAS mutation in PDX models of CXCL12^{high} expressing-PDAC. PA3546 model treated with tipifarnib-atorvastatin-celecoxib combination.

FIG. 10: Administration of tipifarnib treatment regimen (tipifarnib-atorvastatin-celecoxib combination) was more effective at reducing tumor volume in tumors that expressed high levels of CXCL12 in PDX models of KRAS wild type (WT) PDAC.

### DETAILED DESCRIPTION

As used herein, the articles “a,” “an,” and “the” refer to one or to more than one of the grammatical object of the article. By way of example, a sample refers to one sample or two or more samples.

As used herein, the term “subject” refers to a mammal. A subject can be a human or a non-human mammal such as a dog, cat, bovid, equine, mouse, rat, rabbit, or transgenic species thereof. A subject can be a human.

As used herein, the term “sample” refers to a material or mixture of materials containing one or more components of interest. A sample from a subject refers to a sample obtained from the subject, including samples of biological tissue or fluid origin, obtained, reached, or collected in vivo or in situ. A sample can be obtained from a region of a subject containing precancerous or cancer cells or tissues. Such samples can be, but are not limited to, organs, tissues, fractions and cells isolated from a mammal. Exemplary samples include lymph node, whole blood, partially purified blood, serum, plasma, bone marrow, and peripheral blood mononuclear cells (“PBMC”). A sample also can be a tissue biopsy. Exemplary samples also include cell lysate, a cell culture, a cell line, a tissue, oral tissue, gastrointestinal tissue, an organ, an organelle, a biological fluid, a blood sample, a urine sample, a skin sample, and the like.

As used herein, the term “analyzing” a sample refers to carrying that an art-recognized assay to make an assessment regarding a particular property or characteristic of the sample. The property or characteristic of the sample can be, for example, the type of the cells in the sample, or the expression level of a gene in the sample.

As used herein, the terms “treat,” “treating,” and “treatment,” when used in reference to a cancer patient, can refer to an action that reduces the severity of the cancer, or retards or slows the progression of the cancer, including (a) inhibiting the cancer growth, or arresting development of the cancer, or (b) causing regression of the cancer, or (c) delaying, ameliorating or minimizing one or more symptoms associated with the presence of the cancer. For example, “treating” a cancer, such as pancreatic cancer in a subject refers to an action inhibiting the cancer growth in the subject.

As used herein, the term “administer,” “administering,” or “administration” refers to the act of delivering, or causing to be delivered, a compound or a pharmaceutical composition to the body of a subject by a method described herein or otherwise known in the art. Administering a compound or a pharmaceutical composition includes prescribing a compound or a pharmaceutical composition to be delivered into the body of a patient. Exemplary forms of administration include oral dosage forms, such as tablets, capsules, syrups, suspensions; injectable dosage forms, such as intravenous (IV), intramuscular (IM), or intraperitoneal (IP); transdermal dosage forms, including creams, jellies, powders, or patches; buccal dosage forms; inhalation powders, sprays, suspensions, and rectal suppositories.

As used herein, the term “selecting” and “selected” in reference to a subject is used to mean that a particular subject is specifically chosen from a larger group of subjects on the basis of (due to) the particular subject meeting a predetermined criterion or a set of predetermined criteria, e.g., having a lower IGF1 expression than a reference level. Similarly, “selectively treating” a subject refers to providing treatment to a subject meeting a predetermined criterion or a set of predetermined criteria. Similarly, “selectively administering” refers to administering a drug to a subject meeting a predetermined criterion or a set of predetermined criteria. By selecting, selectively treating and selectively administering, it is meant that a subject having a cancer is delivered a personalized therapy based on the subject's biology, rather than being delivered a standard treatment regimen based solely on having the cancer.

As used herein, the term “therapeutically effective amount” of a compound when used in connection with a disease or disorder refers to an amount sufficient to provide a therapeutic benefit in the treatment of the disease or disorder or to delay or minimize one or more symptoms associated with the disease or disorder. The disease or disorder can be a cancer.

As used herein, the term “express” or “expression” when used in connection with a gene refers to the process by which the information carried by the gene becomes manifest as the phenotype, including transcription of the gene to a messenger RNA (mRNA), the subsequent translation of the mRNA molecule to a polypeptide chain and its assembly into the ultimate protein.

As used herein, the term “expression level” of a gene refers to the amount or accumulation of the expression product of the gene, such as, for example, the amount of a RNA product of the gene (the mRNA level of the gene) or the amount of a protein product of the gene (the protein level of the gene). If the gene has more than one allele, the expression level of a gene refers to the total amount of accumulation of the expression product of all existing alleles for this gene, unless otherwise specified. An expression level that is not-detectable means that the expression of the gene cannot be detected by standard assays known in the art to measure such expression.

As used herein, the term “reference” when used in connection with a quantifiable value refers to a predetermined value that one can use to determine the significance of the value as measured in a sample.

As used herein, the term “reference expression level” refers to a predetermined expression level of a gene that one can use to determine the significance of the expression level of the gene in a sample. The sample can be a cell, a group of cells, or a tissue. For example, a reference expression level of a gene can also be a cut-off value determined by a person of ordinary skill in the art through statistical analysis of the expression levels of the gene in various sample cell populations. In some embodiments, the reference expression level of IGF1 can be the median expression level of IGF1 in a population of healthy subjects. In some embodiments, the reference expression level of IGF1 can be the median expression level of IGF1 in a population of subjects having the same type of tumor. For example, the reference expression level for pancreatic cancer patients can be the median expression level of IGF1 in a population of pancreatic cancer patients.

The term “reference ratio” as used herein in connection with the expression levels of two or more genes refers to a ratio predetermined by a person of ordinary skill in the art that can be used to determine the significance of the ratio of the levels of these genes in a sample. The sample can be a cell, a group of cells, or a tissue. For example, a reference ratio of IGFBP7 expression to IGF1R expression can be a predetermined ratio of IGFBP7 expression to IGF1R expression. The reference ratio of the expression levels of two or more genes can be the median ratio of expression levels of these genes in a population of subjects. The reference ratio can also be a cutoff percentile of the expression ratio in a population of subjects having the same type of tumor. The cutoff percentile can be the top 10%, 15%, 20%, 25%, 30%, 35%, 40%, 45%, or 50% cutoff. A reference ratio can also be a cut-off value determined by a person of ordinary skill in the art through, for example, statistical analysis of ratios of expression levels of the two genes in various sample cell populations. In certain embodiments, the reference ratio is 1/10, 1/9, 1/8, 1/7, 1/6, 1/5, 1/4, 1/3, 1/2, 1, 2, 3, 4, 5, 6, 7, 8, 9, 10, 15 or 20. In some embodiments, the reference ratio is 1/10. In some embodiments, the reference ratio is 1/9. In some embodiments, the reference ratio is 1/8. In some embodiments, the reference ratio is 1/7. In some embodiments, the reference ratio is 1/6. In some embodiments, the reference ratio is 1/5. In some embodiments, the reference ratio is 1/4. In some embodiments, the reference ratio is 1/3. In some embodiments, the reference ratio is 1/2. In some embodiments, the reference ratio is 1. In some embodiments, the reference ratio is 2. In some embodiments, the reference ratio is 3. In some embodiments, the reference ratio is 4. In some embodiments, the reference ratio is 5. In some embodiments, the reference ratio is 6. In some embodiments, the reference ratio is 7. In some embodiments, the reference ratio is 8. In some embodiments, the reference ratio is 9. In some embodiments, the reference ratio is 10. In some embodiments, the reference ratio is 15. In some embodiments, the reference ratio is 20.

As used herein, the term “mutation” when used in connection with a gene refers to a change in the DNA sequence of the gene. As well understood in the art, the mutation can be silent that does not result in any change in the amino acid sequence of the corresponding protein. The mutation cause amino acid changes, including conservative and non-conservative changes. The change includes substitutions, deletions, additions, or truncations of the amino acid sequence. A mutant molecule can have one or more mutations. In some embodiments, the genetic alternation in the DNA sequence results in activation of the corresponding protein. Such mutations are referred to as “activating mutations.” As such, an “activating mutation” does not include an alternation in a gene that does not result in the activation of the corresponding protein. Accordingly, a sample or a subject that does not have any “activating mutation” in a particular gene can still have a mutation in the gene that does not affect the activity of the corresponding protein or a mutation that impairs the activity of the protein.

As used herein, the term “mutation allele frequency” or “Variant Allele Frequency (VAF)” when used in connection with a gene in a cancer sample or an individual refers to the proportions of cells that the mutations reside in. For example, the KRAS mutation allele frequency in a subject having a cancer refers to the proportions of the cancer cells in the subject that have a KRAS mutation allele.

As used herein, the term “KRAS wild type cancer” refers to a cancer wherein the KRAS is not mutated or wherein the cancer has a low KRAS Variant Allele Frequency (is or is less than 5%). As used herein, “KRAS” refers to the gene that encodes either of the KRAS4A or KRAS4B isoforms.

As used herein, the term “responsiveness” or “responsive” when used in connection with a treatment refers to the effectiveness of the treatment in lessening or decreasing the symptoms of the disease being treated. In connection with a cancer patient, the patient is responsive to an FTI treatment if the FTI treatment effectively inhibits the growth, or arrests development of the cancer, causes regression of the cancer, or delays or minimizes one or more symptoms associated with the presence of the cancer in the patient.

As used herein, the term “likelihood” refers to the probability of an event. A subject is “likely” to be responsive to a particular treatment when a condition is met means that the probability of the subject to be responsive to a particular treatment is higher when the condition is met than when the condition is not met. The probability to be responsive to a particular treatment can be higher by, for example, 5%, 10%, 25%, 50%, 100%, 200%, or more in a subject who meets a particular condition compared to a subject who does not meet the condition. For example, a subject having a cancer is “likely” responsive to an FTI treatment when the subject has a lower IGF1 expression than a reference ratio means that the probability of a subject to be responsive to FTI treatment is 5%, 10%, 25%, 50%, 100%, 200%, or more higher in a subject who has lower IGF1 expression than a reference ratio compared to a subject who has higher IGF1 expression than the reference ratio.

CXCL12 (or Stroma Derived Factor 1) is a strong chemotactic agent for lymphocytes. During embryogenesis, CXCL12 directs the migration of hematopoietic cells from fetal liver to bone, and in adulthood, CXCL12 plays an important role in angiogenesis by recruiting endothelial progenitor cells through a CXCR4-dependent mechanism. CXCL12 is also expressed within the splenic red pulp and lymph node medullary cords. See Pitt et al., 2015, Cancer Cell 27:755-768 and Zhao et al., 2011, Proc. Natl. Acad. Sci. USA 108:337-342. An exemplary amino acid sequence and a corresponding encoding nucleic acid sequence of human CXCL12 can be found at GENBANK ACCESSION NOS.: NP_000600.1 and NM_000609.6, respectively.

CXCR4 (also known as fusin or CD184) is a receptor specific for CXCL12. The protein has 7 transmembrane regions and is located on the cell surface. It acts with the CD4 protein to support HIV entry into cells and is also highly expressed in breast cancer cells. Alternate transcriptional splice variants, encoding different isoforms, have been characterized. An exemplary amino acid sequence and a corresponding encoding nucleic acid sequence of human CXCR4 can be found at GENBANK ACCESSION NOS.: NP_001008540.1 and NM_001008540.1, respectively.

CXCR2 is a receptor that recognizes CXC chemokines that possess an E-L-R amino acid motif immediately adjacent to their CXC motif. ELR-positive chemokines, such as CXCL1 to CXCL7, bind to CXCR2. CXCR2 is expressed on the surface of neutrophils in mammals. Contrary to CXCR4, CXCR2 plays important roles in stem cell mobilization from the bone marrow to peripheral blood and is known as “mobilizing receptor.” Exemplary encoding nucleic acid sequences of human CXCR2 transcript variants can be found at GENBANK ACCESSION NOS.: NM_001168298.1 and NM_001557.3.

Insulin-like growth factor 1 (IGF1) is similar to insulin in function and structure and is a member of a family of proteins involved in mediating growth and development. The protein is processed from a precursor, bound by a specific receptor, and secreted. Defects in this gene are a cause of IGF1 deficiency. Alternative splicing results in multiple transcript variants encoding different isoforms that may undergo similar processing to generate mature protein. Exemplary amino acid sequences and corresponding encoding nucleic acid sequences of human IGF1 transcript variants can be found at GENBANK ACCESSION NOS.: NM_000618.4, and NP_000609.1; NM_001111283.2, and NP_001104753.1; NM_001111284.1, and NP_001104754.1; or NM_001111285.2, and NP_001104755.1.

Insulin-like growth factor 2 (IGF2) is another member of the insulin family of polypeptide growth factors, which are involved in development and growth. It is an imprinted gene, expressed only from the paternal allele, and epigenetic changes at this locus are associated with a number of human diseases, including Wilms tumour, Beckwith-Wiedemann syndrome, rhabdomyosarcoma, and Silver-Russell syndrome. Loss of imprinting and/or loss of heterozygosity of IGFII are associated with development of cancer, such as ovarian cancer and colorectal cancer. Chen et al., Clinical cancer research 6 (2) (2000): 474-479; Cui et al., Cancer research 62(22) (2002): 6442-6446. A read-through INS-IGF2 gene exists, whose 5′ region overlaps the INS gene and the 3′ region overlaps this gene. Alternatively spliced transcript variants encoding different isoforms have been found for this gene. Exemplary amino acid sequences and corresponding encoding nucleic acid sequences of human IGF2 transcript variants can be found at GENBANK ACCESSION NOS.: NM_000612.5, and NP_000603.1; NM_001007139.5, and NP_001007140.2; NM_001127598.2, and NP_001121070.1; NM_001291861.2, and NP_001278790.1; NM_001291862.2, and NP_001278791.1.

Insulin-like growth factor binding protein 7 (IGFBP7) is a member of the insulin-like growth factor (IGF)-binding protein (IGFBP) family. IGFBPs bind IGFs with high affinity, and regulate IGF availability in body fluids and tissues and modulate IGF binding to its receptors. This protein binds IGFI and IGFII with relatively low affinity, and belongs to a subfamily of low-affinity IGFBPs. It also stimulates prostacyclin production and cell adhesion. Alternatively spliced transcript variants encoding different isoforms have been described for this gene. Exemplary amino acid sequences and corresponding encoding nucleic acid sequences of human IGF1 transcript variants can be found at GENBANK ACCESSION NOS.: NP_001240764.1, and NM_001253835.1; or NM_001553.2, and NP_001544.1.

Insulin-like growth factor 1 receptor (IGF1R) binds insulin-like growth factor with a high affinity. It has tyrosine kinase activity. The IGF1R plays a critical role in transformation events. Cleavage of the precursor generates alpha and beta subunits. It is highly overexpressed in most malignant tissues where it functions as an anti-apoptotic agent by enhancing cell survival. Alternatively spliced transcript variants encoding distinct isoforms have been found for this gene. Exemplary amino acid sequences and corresponding encoding nucleic acid sequences of human IGF1R transcript variants can be found at GENBANK ACCESSION NOS.: NM_000875.4 or NP_000866.1; and NM_001291858.1 or NP_001278787.1.

Insulin-like growth factor 2 receptor (IGF2R) is a receptor for both insulin-like growth factor 2 and mannose 6-phosphate. This receptor has various functions, including in the intracellular trafficking of lysosomal enzymes, the activation of transforming growth factor beta, and the degradation of insulin-like growth factor 2. Mutation or loss of heterozygosity of this gene has been association with risk of hepatocellular carcinoma. The orthologous mouse gene is imprinted and shows exclusive expression from the maternal allele; however, imprinting of the human gene may be polymorphic, as only a minority of individuals showed biased expression from the maternal allele. Exemplary amino acid sequence and corresponding encoding nucleic acid sequence of human IGF2R can be found at GENBANK ACCESSION NOS.: NM_000876.3, and NP_000867.2.

KRAS, an oncogene, and a member of the small GTPase superfamily. A single amino acid substitution is responsible for an activating mutation. The transforming protein that results is implicated in various malignancies, including for example, lung adenocarcinoma, mucinous adenoma, ductal carcinoma of the pancreas and colorectal carcinoma. Alternative splicing leads to variants encoding two isoforms. Exemplary amino acid sequences and corresponding encoding nucleic acid sequences of human IGF1 transcript variants can be found at GENBANK ACCESSION NOS.: NM_004985.4, and NP_004976.2 (isoform b); and NM_033360.3 and NP_203524.1 (isoform a).

Tumor protein 53 (TP53), a tumor suppressor protein containing transcriptional activation, DNA binding, and oligomerization domains. The encoded protein responds to diverse cellular stresses to regulate expression of target genes, thereby inducing cell cycle arrest, apoptosis, senescence, DNA repair, or changes in metabolism. Mutations in this gene are associated with a variety of human cancers, including hereditary cancers such as Li-Fraumeni syndrome. Alternative splicing of this gene and the use of alternate promoters result in multiple transcript variants and isoforms. Additional isoforms have also been shown to result from the use of alternate translation initiation codons from identical transcript variants. Exemplary amino acid sequences and corresponding encoding nucleic acid sequences of human TP53 transcript variants can be found at GENBANK ACCESSION NOS.: NM_000546.5, and NP_000537.3; NM_001126112.2, and NP_001119584.1; or NM_001126113.2 and NP_001119585.1.

Phosphatidylinositol 3-kinase (PI3K) is composed of an 85 kDa regulatory subunit and a 110 kDa catalytic subunit. Phosphatidylinositol-4,5-bisphosphate 3-kinase catalytic subunit alpha (PIK3CA) is the catalytic subunit, which uses ATP to phosphorylate PtdIns, PtdIns4P and PtdIns(4,5)P2. This gene has been found to be oncogenic and its mutation has been implicated in a number of human cancers. Martini et al., Annals of Medicine, 46 (6) 372-383 (2014). Exemplary amino acid sequences and corresponding encoding nucleic acid sequences of human PIK3CA transcript variants can be found at GENBANK ACCESSION NOS.: XM 006713658.4 and XP 006713721.1; or XM 011512894.2 and XP 0115111961

AKT serine/threonine kinase 1 (AKT), a serine-threonine protein kinase encoded by the AKT1 gene, is catalytically inactive in serum-starved primary and immortalized fibroblasts. AKT1 and the related AKT2 are activated by platelet-derived growth factor. The activation is rapid and specific, and it is abrogated by mutations in the pleckstrin homology domain of AKT1. It was shown that the activation occurs through PI3K. AKT mutation has also been implicated in a number of human cancers. Martini et al. (2014). Multiple alternatively spliced transcript variants have been found for this gene. Exemplary amino acid sequences and corresponding encoding nucleic acid sequences of human AKT transcript variants can be found at GENBANK ACCESSION NOS.: NM_001014431.1 and NP_001014431.1; NM_001014432.1 and NP_001014432.1; or NM_005163.2 and NP_005154.2.

### 1. METHODS

Provided herein are methods for selecting a subject having a cancer for treatment with an FTI. The methods provided herein are based, in part, on the discovery that cancer patients with different gene expression level, mutation status, or clinical presentation respond differently to an FTI treatment, and that the clinical benefits FTIs of treatment are associated with the gene expression levels and/or mutation status of certain genes, or clinical presentation of the cancer. Disclosed herein is the discovery of the crosstalk between the IGF1R and CXCL12/CXCR4 pathways that defines objective responses to the FTI. Specifically, it was found that the IGF1 pathway mediates resistance to FTI (e.g. tipifarnib), while the CXCL12/CXCR4 pathway activation can predict objectives responses. As such, patients having cancer that is characterized by an active CXCL12/CXCR4 pathway and inactive IGF1R pathway are likely responsive to an FTI treatment, and selection of such patients for an FTI treatment can increase the overall response rate of the treatment.

### 1.1. Biomarkers

As such, provided herein are methods to select cancer patients for an FTI treatment, methods to predict the responsiveness to an FTI treatment in a cancer patient, and methods to increase the overall responsiveness of an FTI treatment in a cancer patient population, based on an active CXCL12/CXCR4 pathway and an inactive IGF1R pathway. The CXCL12/CXCR4 pathway activity is relatively high when (a) the CXCL12 expression level is relatively high, (b) the expression ratio of CXCL12 to CXCR4 is relatively high, and/or (c) the CXCR4 expression is relatively high. Thus, subjects with an active CXCL12/CXCR4 pathway can have a greater CXCL12 expression level than a reference level of CXCL12 expression, a greater expression ration of CXCL12 to CXCR4 than a reference ratio, or a greater CXCR4 expression level than a reference level of CXCR4 expression.

IGF1R pathway can be activated by the binding of IGF1 to IGF1R, and inhibited by binding proteins such as IGFBP7. Thus, the activity of IGF1R pathway is relatively low in a cancer with a relatively low level of IGF1 expression. The activity of IGF1R pathway can be relatively low in a cancer irrespective of the IGF1 expression, if the IGFBP7 expression level is relatively high. As the IGF1R pathway can be activated by activating mutation of the IGF1R pathway proteins, such as PI3K or AKT, subjects with inactive IGF1R pathway can also be selected based on the absence of activating mutation in PI3K or AKT. The activating mutation in PI3K can be an activating mutation in PIK3CA. Thus, subjects with inactive IGF1R pathway can also be selected based on the absence of activating mutation in PI3K or AKT

In some embodiments, provided herein are methods of treating cancer in a subject by administering a therapeutically effective amount of FTI to the subject, wherein the subject has a greater CXCL12 expression than a reference level of CXCL12 expression, and a lower IGF1 expression than a reference level of IGF1 expression. In some embodiments, the subject has an IGF1 expression that is non-detectable. In some embodiments, provided herein are methods of treating cancer in a subject by administering a therapeutically effective amount of FTI to the subject, wherein the subject has a greater CXCL12 expression than a reference level of CXCL12 expression, and a greater IGFBP7 expression than a reference level of IGFBP7 expression.

Also disclosed herein is the finding that the IGFBP7 variant L11F (rs11573021) can indicate resistance of a cancer to an FTI treatment. Accordingly, in some embodiments, the subject selected for an FTI treatment further does not carry the IGFBP7 variant L11F (rs11573021). In some embodiments, provided herein are methods of treating cancer in a subject by administering a therapeutically effective amount of FTI to the subject, wherein the subject has a greater CXCL12 expression than a reference level of CXCL12 expression, and wherein the subject further does not carry the IGFBP7 variant L11F (rs11573021). In some embodiments, provided herein are methods of treating cancer in a subject by administering a therapeutically effective amount of FTI to the subject, wherein the subject has a greater CXCL12 expression than a reference level of CXCL12 expression, and wherein the subject further does not carry an activating mutation in PIK3CA. In some embodiments, provided herein are methods of treating cancer in a subject by administering a therapeutically effective amount of FTI to the subject, wherein the subject has a greater CXCL12 expression than a reference level of CXCL12 expression, and wherein the subject further does not carry an activating mutation in AKT.

In some embodiments, provided herein are methods of treating cancer in a subject by administering a therapeutically effective amount of FTI to the subject, wherein the subject has a greater CXCL12 expression than a reference level of CXCL12 expression, and wherein the subject has (1) a lower IGF1 expression than a reference level of IGF1 expression, or (2) a greater IGFBP7 expression than a reference level of IGFBP7 expression, wherein the subject further does not carry the IGFBP7 variant L11F (rs11573021), an activating mutation in PIK3CA, and/or activating mutation in AKT.

IGF2 is normally silenced by imprinting. Loss of imprinting (LOI) and loss of heterozygosity (LOH) are common alterations in cancer that typically involve the activation of the normally silent maternal allele of the IGF2 gene. LOI or LOH of IGF2 can lead to increased IGF2 expression and activation of the IGF1R pathway by binding of IGF2 to IGF1R, which can result in resistance to FTI treatment. IGF2 can be inactivated by IGF2R, which mediates the degradation of IGF2. As such, the activity of IGF1R pathway can be relatively low in a cancer even with a relatively high level of IGF2 expression, if the IGF2R level is relatively high.

Accordingly, the methods provided herein for selecting subjects having a cancer for an FTI treatment further include measuring IGF2 expression in the subject. In some embodiments, provided herein are methods of treating cancer in a subject by administering a therapeutically effective amount of FTI to the subject, wherein the subject has a greater CXCL12 expression than a reference level of CXCL12 expression, and a lower IGF2 expression than a reference level of IGF2 expression. In some embodiments, the subject has an IGF2 expression that is non-detectable. In some embodiments, provided herein are methods of treating cancer in a subject by administering a therapeutically effective amount of FTI to the subject, wherein the subject has a greater CXCL12 expression than a reference level of CXCL12 expression, and a greater IGF2R expression than a reference level of IGF2R expression. In some embodiments, the subject does not have a loss of heterozygosity of IGF2. In some embodiments, the subject does not have a loss of imprinting of IGF2. In some embodiments, the subject does not have a loss of heterozygosity of IGF2 or a loss of imprinting of IGF2.

In some embodiments, methods provided herein use both IGF1 and IGF2 expression levels to select cancer patients for an FTI treatment. In some embodiments, provided herein are methods of treating cancer in a subject by administering a therapeutically effective amount of FTI to the subject, wherein the subject has a greater CXCL12 expression than a reference level of CXCL12 expression, a lower IGF1 expression than a reference level of IGF1 expression, and a lower IGF2 expression than a reference level of IGF2 expression. In some embodiments, the subject has an IGF1 expression that is non-detectable. In some embodiments, the subject has an IGF2 expression that is non-detectable. In some embodiments, the subject has an IGF1 expression that is non-detectable and an IGF2 expression that is non-detectable.

In addition to CXCL12 expression level, the expression ratio of CXCL12 to CXCR4 also indicates the activation of the CXCL12/CXCR4 pathway, and can predict the likelihood of responsiveness of a cancer to an FTI treatment. As such, provided herein are also methods to select cancer patients for an FTI treatment, methods to predict the responsiveness of an FTI treatment in a cancer patient, and methods to increase the responsiveness to an FTI treatment in a cancer patient population based the expression ratio of CXCL12 to CXCR4. In some embodiments, provided herein are methods of treating cancer in a subject by administering a therapeutically effective amount of an FTI to the subject, wherein the subject has a greater expression ratio of CXCL12 to CXCR4 than a reference ratio, and an inactive IGF1R pathway.

The subject having an inactive IGF1R pathway can have a lower IGF1 expression than a reference level of IGF1 expression, or a greater IGFBP7 expression than a reference level of IGFBP7 expression. In some embodiments, provided herein are methods of treating cancer in a subject by administering a therapeutically effective amount of an FTI to the subject, wherein the subject has a greater expression ratio of CXCL12 to CXCR4 than a reference ratio, and a lower IGF1 expression than a reference level of IGF1 expression. In some embodiments, the subject has an IGF1 expression that is non-detectable. In some embodiments, provided herein are methods of treating cancer in a subject by administering a therapeutically effective amount of an FTI to the subject, wherein the subject has a greater expression ratio of CXCL12 to CXCR4 than a reference ratio, and a greater IGFBP7 expression than a reference level of IGFBP7 expression.

In some embodiments, provided herein are methods of treating cancer in a subject by administering a therapeutically effective amount of FTI to the subject, wherein the subject has a greater expression ratio of CXCL12 to CXCR4 than a reference ratio, and wherein the subject further does not carry the IGFBP7 variant L11F (rs11573021). In some embodiments, provided herein are methods of treating cancer in a subject by administering a therapeutically effective amount of FTI to the subject, wherein the subject has a greater expression ratio of CXCL12 to CXCR4 than a reference ratio, and wherein the subject further does not carry an activating mutation in PIK3CA. In some embodiments, provided herein are methods of treating cancer in a subject by administering a therapeutically effective amount of FTI to the subject, wherein the subject has a greater expression ratio of CXCL12 to CXCR4 than a reference ratio, and wherein the subject further does not carry an activating mutation in AKT.

In some embodiments, provided herein are methods of treating cancer in a subject by administering a therapeutically effective amount of FTI to the subject, wherein the subject has a greater expression ratio of CXCL12 to CXCR4 than a reference ratio, and wherein the subject has (1) a lower IGF1 expression than a reference level of IGF1 expression, or (2) a greater IGFBP7 expression than a reference level of IGFBP7 expression, wherein the subject further does not carry the IGFBP7 variant L11F (rs11573021), an activating mutation in PIK3CA, and/or activating mutation in AKT.

In some embodiments, the subjects selected for an FTI treatment based on a greater expression ratio of CXCL12 to CXCR4 than a reference ratio further has a lower IGF2 expression than a reference level of IGF2 expression. In some embodiments, the subject has an IGF2 expression that is non-detectable. In some embodiments, provided herein are methods of treating cancer in a subject by administering a therapeutically effective amount of an FTI to the subject, wherein the subject has a greater expression ratio of CXCL12 to CXCR4, and a greater IGF2R expression than a reference level of IGF2R expression. In some embodiments, the subject does not have a loss of heterozygosity of IGF2. In some embodiments, the subject does not have a loss of imprinting of IGF2. In some embodiments, the subject does not have a loss of heterozygosity of IGF2 or a loss of imprinting of IGF2.

In some embodiments, the subjects selected for an FTI treatment based on a greater expression ratio of CXCL12 to CXCR4 than a reference ratio further has a lower IGF1 expression than a reference level of IGF1 expression, and a lower IGF2 expression than a reference level of IGF2 expression. In some embodiments, the subject has an IGF1 expression that is non-detectable. In some embodiments, the subject has an IGF2 expression that is non-detectable. In some embodiments, the subject has an IGF1 expression that is non-detectable and an IGF2 expression that is non-detectable.

In addition, the activation level of CXCR4 can also predict the likelihood of responsiveness of a cancer to an FTI treatment. As such, provided herein are also methods to select cancer patients for an FTI treatment, methods to predict the responsiveness of an FTI treatment in a cancer patient, and methods to increase the responsiveness to an FTI treatment in a cancer patient population based the expression level of CXCR4. In some embodiments, provided herein are methods of treating cancer in a subject by administering a therapeutically effective amount of an FTI to the subject, wherein the subject has a greater expression level of CXCR4 than a reference expression level of CXCR4, and an inactive IGF1R pathway. In some embodiments, provided herein are methods of treating cancer in a subject by administering a therapeutically effective amount of an FTI to the subject, wherein the subject has a greater expression ratio of CXCR4 to CXCR2 than a reference ratio, and an inactive IGF1R pathway. In some embodiments, provided herein are methods of treating cancer in a subject by administering a therapeutically effective amount of an FTI to the subject, wherein the subject has an activating mutation in CXCR4, and an inactive IGF1R pathway.

The subject having an inactive IGF1R pathway can have a lower IGF1 expression than a reference level of IGF1 expression. In some embodiments, provided herein are methods of treating cancer in a subject by administering a therapeutically effective amount of an FTI to the subject, wherein the subject has a greater expression level of CXCR4 than a reference expression level of CXCR4, and a lower IGF1 expression than a reference level of IGF1 expression. In some embodiments, provided herein are methods of treating cancer in a subject by administering a therapeutically effective amount of an FTI to the subject, wherein the subject has a greater expression ratio of CXCR4 to CXCR2 than a reference expression ratio, and a lower IGF1 expression than a reference level of IGF1 expression. In some embodiments, provided herein are methods of treating cancer in a subject by administering a therapeutically effective amount of an FTI to the subject, wherein the subject has an activating mutation of CXCR4, and a lower IGF1 expression than a reference level of IGF1 expression. In some embodiments, the subject has an IGF1 expression that is non-detectable.

In other embodiments, the subject having an inactive IGF1R pathway can be a greater IGFBP7 expression than a reference level of IGFBP7 expression. In some embodiments, provided herein are methods of treating cancer in a subject by administering a therapeutically effective amount of an FTI to the subject, wherein the subject has a greater expression level of CXCR4 than a reference expression level of CXCR4, and a greater IGFBP7 expression than a reference level of IGFBP7 expression. In some embodiments, provided herein are methods of treating cancer in a subject by administering a therapeutically effective amount of an FTI to the subject, wherein the subject has a greater expression ratio of CXCR4 to CXCR2 than a reference expression ratio, and a greater IGFBP7 expression than a reference level of IGFBP7 expression. In some embodiments, provided herein are methods of treating cancer in a subject by administering a therapeutically effective amount of an FTI to the subject, wherein the subject has an activating mutation of CXCR4, and a greater IGFBP7 expression than a reference level of IGFBP7 expression.

In some embodiments, the subjects selected for an FTI treatment based on a greater expression ratio of CXCL12 to CXCR4 than a reference ratio further has a lower IGF2 expression than a reference level of IGF2 expression. In some embodiments, the subject has an IGF2 expression that is non-detectable. In some embodiments, provided herein are methods of treating cancer in a subject by administering a therapeutically effective amount of an FTI to the subject, wherein the subject has a greater expression ratio of CXCL12 to CXCR4, and a greater IGF2R expression than a reference level of IGF2R expression. In some embodiments, the subject does not have a loss of heterozygosity of IGF2. In some embodiments, the subject does not have a loss of imprinting of IGF2. In some embodiments, the subject does not have a loss of heterozygosity of IGF2 or a loss of imprinting of IGF2.

In some embodiments, the subjects selected for an FTI treatment based on a greater expression ratio of CXCL12 to CXCR4 than a reference ratio further has a lower IGF1 expression than a reference level of IGF1 expression, and a lower IGF2 expression than a reference level of IGF2 expression. In some embodiments, the subject has an IGF1 expression that is non-detectable. In some embodiments, the subject has an IGF2 expression that is non-detectable. In some embodiments, the subject has an IGF1 expression that is non-detectable and an IGF2 expression that is non-detectable.

Disclosed herein is also the finding that KRAS activity mediates FTI resistance. As such, provided herein are methods to select cancer patients for an FTI treatment, methods to predict the responsiveness of an FTI treatment in a cancer patient, and methods to increase the responsiveness to an FTI treatment in a cancer patient population based the mutation status of KRAS. In some embodiments, provided herein are methods of treating cancer in a subject by administering a therapeutically effective amount of an FTI to the subject, wherein the subject has a low KRAS mutation allele frequency (Variant Allele Frequency, VAF). In some embodiments, the subject has a KRAS mutation allele frequency that is less than 15%. In some embodiments, the subject has a KRAS mutation allele frequency that is less than 12%. In some embodiments, the subject has a KRAS mutation allele frequency that is less than 10%. In some embodiments, the subject has a KRAS mutation allele frequency that is less than 8%. In some embodiments, the subject has a KRAS mutation allele frequency that is less than 7%. In some embodiments, the subject has a KRAS mutation allele frequency that is less than 6%. In some embodiments, the subject has a KRAS mutation allele frequency that is or less than 5%. In some embodiments, the subject does not have an activating mutation in KRAS. In some embodiments, the KRAS status is assessed at primary diagnosis or in recurrent or metastatic disease. In some embodiments, if several samples are available, KRAS testing should be performed in the most recently obtained tumor sample.

In some embodiments, provided herein are methods of treating cancer in a subject by administering a therapeutically effective amount of an FTI to the subject, wherein the subject has a greater CXCL12 expression than a reference level of CXCL12 expression, and wherein the KRAS mutation allele frequency (Variant Allele Frequency, VAF) is or is less than 5%.

Disclosed herein is also the finding that TP53 activity mediates FTI resistance. As such, provided herein are methods to select cancer patients for an FTI treatment, methods to predict the responsiveness of an FTI treatment in a cancer patient, and methods to increase the responsiveness to an FTI treatment in a cancer patient population based the mutation status of TP53. In some embodiments, provided herein are methods of treating cancer in a subject by administering a therapeutically effective amount of an FTI to the subject, wherein the subject has a low TP53 mutation allele frequency. In some embodiments, the subject has a TP53 mutation allele frequency that is less than 15%. In some embodiments, the subject has a TP53 mutation allele frequency that is less than 12%. In some embodiments, the subject has a TP53 mutation allele frequency that is less than 10%. In some embodiments, the subject has a TP53 mutation allele frequency that is less than 8%. In some embodiments, the subject has a TP53 mutation allele frequency that is less than 7%. In some embodiments, the subject has a TP53 mutation allele frequency that is less than 6%. In some embodiments, the subject has a TP53 mutation allele frequency that is or less than 5%. In some embodiments, the subject does not have an activating mutation in the TP53 gene.

In some embodiments, provided herein are methods to select cancer patients for an FTI treatment, methods to predict the responsiveness of an FTI treatment in a cancer patient, and methods to increase the responsiveness to an FTI treatment in a cancer patient population based the mutation status of KRAS and TP53. In some embodiments, provided herein are methods of treating cancer in a subject by administering a therapeutically effective amount of an FTI to the subject, wherein the subject has a low KRAS mutation allele frequency and a low TP53 mutation allele frequency. In some embodiments, the subject has a TP53 mutation allele frequency that is less than 15% and a KRAS mutation allele frequency that is less than 15%. In some embodiments, the subject has a TP53 mutation allele frequency that is less than 12% and a KRAS mutation allele frequency that is less than 12%. In some embodiments, the subject has a TP53 mutation allele frequency that is less than 10% and a KRAS mutation allele frequency that is less than 10%. In some embodiments, the subject has a TP53 mutation allele frequency that is less than 8% and a KRAS mutation allele frequency that is less than 8%. In some embodiments, the subject has a TP53 mutation allele frequency that is less than 7% and a KRAS mutation allele frequency that is less than 7%. In some embodiments, the subject has a TP53 mutation allele frequency that is less than 6% and a KRAS mutation allele frequency that is less than 6%. In some embodiments, the subject has a TP53 mutation allele frequency that is less than 5% and a KRAS mutation allele frequency that is less than 5%. In some embodiment, the subject has In some embodiments, the subject does not have an activating mutation in KRAS or TP53.

As a person skill in the art would understand, the markers for patient selection for an FTI treatment as described herein can be used independently or in any combination. As disclosed herein, both CXCL12 and CXCR4 can drive the sensitivity of a cancer to an FTI treatment, and factors including, for example, IGF1R pathway activity and the mutation status of TP53 or KRAS can confer resistance to an FTI treatment. Accordingly, for example, provided herein are methods for selecting cancer patients for an FTI treatment wherein the patients have (1) either CXCL12 activation (high CXCL12 expression level or high CXCL12/CXCR4 ratio) or CXCR4 activation (high CXCR4 level, high CXCR4/CXCR2 ratio, or activating mutation in CXCR4), and (2) inactive IGF1R pathway (low IGF1 expression, low IGF2 expression, high IGFBP7 expression, and/or absence of activating mutation in PIK3CA and AKT); and low mutation allele frequency for TP53 and/or KRAS.

The FTI can be any FTI, including those described herein. For example, the FTI can be tipifarnib, lonafarnib, arglabin, perrilyl alcohol, L778123, L739749, FTI-277, L744832, CP-609,754, R208176, AZD3409, or BMS-214662. In some embodiments, the FTI is tipifarnib. As such, provided herein are methods to select cancer patients for tipifarnib treatment, methods to predict the responsiveness to tipifarnib treatment in a cancer patient, and methods to increase the overall responsiveness of tipifarnib treatment in a cancer patient population, based on an active CXCL12/CXCR4 pathway and an inactive IGF1R pathway. The CXCL12/CXCR4 pathway activity is relatively high when the CXCL12 expression level is relatively high, the expression ratio of CXCL12 to CXCR4 is relatively high, or the CXCR4 expression level is relatively high. Thus, subjects with an active CXCL12/CXCR4 pathway can have a greater CXCL12 expression level than a reference level of CXCL12 expression, a greater expression ration of CXCL12 to CXCR4 than a reference ratio, or a greater CXCR4 expression level than a reference level of CXCR4 expression.

In some embodiments, provided herein are methods of treating cancer in a subject by administering a therapeutically effective amount of tipifarnib to the subject, wherein the subject has a greater CXCL12 expression than a reference level of CXCL12 expression, and a lower IGF1 expression than a reference level of IGF1 expression. In some embodiments, the subject has an IGF1 expression that is non-detectable. In some embodiments, provided herein are methods of treating cancer in a subject by administering a therapeutically effective amount of tipifarnib to the subject, wherein the subject has a greater CXCL12 expression than a reference level of CXCL12 expression, and a greater IGFBP7 expression than a reference level of IGFBP7 expression.

In some embodiments, the subject selected for tipifarnib treatment further does not carry the IGFBP7 variant L11F (rs11573021). In some embodiments, provided herein are methods of treating cancer in a subject by administering a therapeutically effective amount of tipifarnib to the subject, wherein the subject has a greater CXCL12 expression than a reference level of CXCL12 expression, and wherein the subject further does not carry the IGFBP7 variant L11F (rs11573021). In some embodiments, provided herein are methods of treating cancer in a subject by administering a therapeutically effective amount of tipifarnib to the subject, wherein the subject has a greater CXCL12 expression than a reference level of CXCL12 expression, and wherein the subject further does not carry an activating mutation in PIK3CA. In some embodiments, provided herein are methods of treating cancer in a subject by administering a therapeutically effective amount of tipifarnib to the subject, wherein the subject has a greater CXCL12 expression than a reference level of CXCL12 expression, and wherein the subject further does not carry an activating mutation in AKT.

In some embodiments, provided herein are methods of treating cancer in a subject by administering a therapeutically effective amount of tipifarnib to the subject, wherein the subject has a greater CXCL12 expression than a reference level of CXCL12 expression, and wherein the subject has (1) a lower IGF1 expression than a reference level of IGF1 expression, or (2) a greater IGFBP7 expression than a reference level of IGFBP7 expression, wherein the subject further does not carry the IGFBP7 variant L11F (rs11573021), an activating mutation in PIK3CA, and/or activating mutation in AKT.

In some embodiments, provided herein are methods of treating cancer in a subject by administering a therapeutically effective amount of tipifarnib to the subject, wherein the subject has a greater CXCL12 expression than a reference level of CXCL12 expression, and a lower IGF2 expression than a reference level of IGF2 expression. In some embodiments, the subject has an IGF2 expression that is non-detectable. In some embodiments, provided herein are methods of treating cancer in a subject by administering a therapeutically effective amount of tipifarnib to the subject, wherein the subject has a greater CXCL12 expression than a reference level of CXCL12 expression, and a greater IGF2R expression than a reference level of IGF2R expression. In some embodiments, the subject does not have a loss of heterozygosity of IGF2. In some embodiments, the subject does not have a loss of imprinting of IGF2. In some embodiments, the subject does not have a loss of heterozygosity of IGF2 or a loss of imprinting of IGF2.

In some embodiments, methods provided herein use both IGF1 and IGF2 expression levels to select cancer patients for tipifarnib treatment. In some embodiments, provided herein are methods of treating cancer in a subject by administering a therapeutically effective amount of tipifarnib to the subject, wherein the subject has a greater CXCL12 expression than a reference level of CXCL12 expression, a lower IGF1 expression than a reference level of IGF1 expression, and a lower IGF2 expression than a reference level of IGF2 expression. In some embodiments, the subject has an IGF1 expression that is non-detectable. In some embodiments, the subject has an IGF2 expression that is non-detectable. In some embodiments, the subject has an IGF1 expression that is non-detectable and an IGF2 expression that is non-detectable.

In some embodiments, provided herein are methods of treating cancer in a subject by administering a therapeutically effective amount of tipifarnib to the subject, wherein the subject has a greater expression ratio of CXCL12 to CXCR4 than a reference ratio, and an inactive IGF1R pathway. In some embodiments, the reference ratio is 1/10, 1/9, 1/8, 1/7, 1/6, 1/5, 1/4, 1/3, 1/2, 1, 2, 3, 4, 5, 6, 7, 8, 9, 10, or any numeric value between 1/10 and 10. In some embodiments, provided herein are methods of treating cancer in a subject by administering a therapeutically effective amount of tipifarnib to the subject, wherein the subject has a greater expression ratio of CXCL12 to CXCR4 than a reference ratio, and a lower IGF1 expression than a reference level of IGF1 expression. In some embodiments, the subject has an IGF1 expression that is non-detectable. In some embodiments, provided herein are methods of treating cancer in a subject by administering a therapeutically effective amount of tipifarnib to the subject, wherein the subject has a greater expression ratio of CXCL12 to CXCR4 than a reference ratio, and a greater IGFBP7 expression than a reference level of IGFBP7 expression.

In some embodiments, provided herein are methods of treating cancer in a subject by administering a therapeutically effective amount of tipifarnib to the subject, wherein the subject has a greater expression ratio of CXCL12 to CXCR4 than a reference ratio, and wherein the subject further does not carry the IGFBP7 variant L11F (rs11573021). In some embodiments, provided herein are methods of treating cancer in a subject by administering a therapeutically effective amount of tipifarnib to the subject, wherein the subject has a greater expression ratio of CXCL12 to CXCR4 than a reference ratio, and wherein the subject further does not carry an activating mutation in PIK3CA. In some embodiments, provided herein are methods of treating cancer in a subject by administering a therapeutically effective amount of tipifarnib to the subject, wherein the subject has a greater expression ratio of CXCL12 to CXCR4 than a reference ratio, and wherein the subject further does not carry an activating mutation in AKT.

In some embodiments, provided herein are methods of treating cancer in a subject by administering a therapeutically effective amount of tipifarnib to the subject, wherein the subject has a greater expression ratio of CXCL12 to CXCR4 than a reference ratio, and wherein the subject has (1) a lower IGF1 expression than a reference level of IGF1 expression, or (2) a greater IGFBP7 expression than a reference level of IGFBP7 expression, wherein the subject further does not carry the IGFBP7 variant L11F (rs11573021), an activating mutation in PIK3CA, and/or activating mutation in AKT.

In some embodiments, the subjects selected for tipifarnib treatment based on a greater expression ratio of CXCL12 to CXCR4 than a reference ratio further has a lower IGF2 expression than a reference level of IGF2 expression. In some embodiments, the subject has an IGF2 expression that is non-detectable. In some embodiments, provided herein are methods of treating cancer in a subject by administering a therapeutically effective amount of tipifarnib to the subject, wherein the subject has a greater expression ratio of CXCL12 to CXCR4, and a greater IGF2R expression than a reference level of IGF2R expression. In some embodiments, the subject does not have a loss of heterozygosity of IGF2. In some embodiments, the subject does not have a loss of imprinting of IGF2. In some embodiments, the subject does not have a loss of heterozygosity of IGF2 or a loss of imprinting of IGF2.

In some embodiments, the subjects selected for tipifarnib treatment based on a greater expression ratio of CXCL12 to CXCR4 than a reference ratio further has a lower IGF1 expression than a reference level of IGF1 expression, and a lower IGF2 expression than a reference level of IGF2 expression. In some embodiments, the subject has an IGF1 expression that is non-detectable. In some embodiments, the subject has an IGF2 expression that is non-detectable. In some embodiments, the subject has an IGF1 expression that is non-detectable and an IGF2 expression that is non-detectable.

In addition, the activity level of CXCR4 can also predict the likelihood of responsiveness of a cancer to tipifarnib treatment. As such, provided herein are also methods to select cancer patients for tipifarnib treatment, methods to predict the responsiveness of tipifarnib treatment in a cancer patient, and methods to increase the responsiveness to tipifarnib treatment in a cancer patient population based the expression level of CXCR4. In some embodiments, provided herein are methods of treating cancer in a subject by administering a therapeutically effective amount of tipifarnib to the subject, wherein the subject has a greater expression level of CXCR4 than a reference expression level of CXCR4, and an inactive IGF1R pathway. In some embodiments, provided herein are methods of treating cancer in a subject by administering a therapeutically effective amount of tipifarnib to the subject, wherein the subject has a greater expression ratio of CXCR4 to CXCR2 than a reference ratio, and an inactive IGF1R pathway. In some embodiments, provided herein are methods of treating cancer in a subject by administering a therapeutically effective amount of tipifarnib to the subject, wherein the subject has an activating mutation in CXCR4, and an inactive IGF1R pathway.

The subject having an inactive IGF1R pathway can have a lower IGF1 expression than a reference level of IGF1 expression. In some embodiments, provided herein are methods of treating cancer in a subject by administering a therapeutically effective amount of tipifarnib to the subject, wherein the subject has a greater expression level of CXCR4 than a reference expression level of CXCR4, and a lower IGF1 expression than a reference level of IGF1 expression. In some embodiments, provided herein are methods of treating cancer in a subject by administering a therapeutically effective amount of tipifarnib to the subject, wherein the subject has a greater expression ratio of CXCR4 to CXCR2 than a reference expression ratio, and a lower IGF1 expression than a reference level of IGF1 expression. In some embodiments, provided herein are methods of treating cancer in a subject by administering a therapeutically effective amount of tipifarnib to the subject, wherein the subject has an activating mutation of CXCR4, and a lower IGF1 expression than a reference level of IGF1 expression. In some embodiments, the subject has an IGF1 expression that is non-detectable.

The subject having an inactive IGF1R pathway can be a greater IGFBP7 expression than a reference level of IGFBP7 expression. In some embodiments, provided herein are methods of treating cancer in a subject by administering a therapeutically effective amount of tipifarnib to the subject, wherein the subject has a greater expression level of CXCR4 than a reference expression level of CXCR4, and a greater IGFBP7 expression than a reference level of IGFBP7 expression. In some embodiments, provided herein are methods of treating cancer in a subject by administering a therapeutically effective amount of tipifarnib to the subject, wherein the subject has a greater expression ratio of CXCR4 to CXCR2 than a reference expression ratio, and a greater IGFBP7 expression than a reference level of IGFBP7 expression. In some embodiments, provided herein are methods of treating cancer in a subject by administering a therapeutically effective amount of tipifarnib to the subject, wherein the subject has an activating mutation of CXCR4, and a greater IGFBP7 expression than a reference level of IGFBP7 expression.

In some embodiments, the reference ratio is 1/10, 1/9, 1/8, 1/7, 1/6, 1/5, 1/4, 1/3, 1/2, 1, 2, 3, 4, 5, 6, 7, 8, 9, 10, or any numeric value between 1/10 and 10.

In some embodiments, the subjects selected for tipifarnib treatment based on a greater expression ratio of CXCL12 to CXCR4 than a reference ratio further has a lower IGF2 expression than a reference level of IGF2 expression. In some embodiments, the subject has an IGF2 expression that is non-detectable. In some embodiments, provided herein are methods of treating cancer in a subject by administering a therapeutically effective amount of tipifarnib to the subject, wherein the subject has a greater expression ratio of CXCL12 to CXCR4, and a greater IGF2R expression than a reference level of IGF2R expression. In some embodiments, the subject does not have a loss of heterozygosity of IGF2. In some embodiments, the subject does not have a loss of imprinting of IGF2. In some embodiments, the subject does not have a loss of heterozygosity of IGF2 or a loss of imprinting of IGF2.

In some embodiments, the subjects selected for tipifarnib treatment based on a greater expression ratio of CXCL12 to CXCR4 than a reference ratio further has a lower IGF1 expression than a reference level of IGF1 expression, and a lower IGF2 expression than a reference level of IGF2 expression. In some embodiments, the subject has an IGF1 expression that is non-detectable. In some embodiments, the subject has an IGF2 expression that is non-detectable. In some embodiments, the subject has an IGF1 expression that is non-detectable and an IGF2 expression that is non-detectable.

Disclosed herein is also the finding that KRAS activity mediates tipifarnib resistance. As such, provided herein are methods to select cancer patients for tipifarnib treatment, methods to predict the responsiveness of tipifarnib treatment in a cancer patient, and methods to increase the responsiveness to tipifarnib treatment in a cancer patient population based the mutation status of KRAS. In some embodiments, provided herein are methods of treating cancer in a subject by administering a therapeutically effective amount of tipifarnib to the subject, wherein the subject has a low KRAS mutation allele frequency (Variant Allele Frequency, VAF). In some embodiments, the subject has a KRAS mutation allele frequency that is less than 15%. In some embodiments, the subject has a KRAS mutation allele frequency that is less than 12%. In some embodiments, the subject has a KRAS mutation allele frequency that is less than 10%. In some embodiments, the subject has a KRAS mutation allele frequency that is less than 8%. In some embodiments, the subject has a KRAS mutation allele frequency that is less than 7%. In some embodiments, the subject has a KRAS mutation allele frequency that is less than 6%. In some embodiments, the subject has a KRAS mutation allele frequency that is or less than 5%. In some embodiments, the subject does not have an activating mutation in KRAS. In some embodiments, if several samples are available, KRAS testing should be performed in the most recently obtained tumor sample.

In some embodiments, provided herein are methods of treating cancer in a subject by administering a therapeutically effective amount of tipifarnib to the subject, wherein the subject has a greater CXCL12 expression than a reference level of CXCL12 expression, and wherein the KRAS mutation allele frequency (Variant Allele Frequency, VAF) is or is less than 5%.

Disclosed herein is also the finding that TP53 mutation mediates tipifarnib resistance. As such, provided herein are methods to select cancer patients for tipifarnib treatment, methods to predict the responsiveness of tipifarnib treatment in a cancer patient, and methods to increase the responsiveness to tipifarnib treatment in a cancer patient population based the mutation status of TP53. In some embodiments, provided herein are methods of treating cancer in a subject by administering a therapeutically effective amount of tipifarnib to the subject, wherein the subject has a low TP53 mutation allele frequency. In some embodiments, the subject has a TP53 mutation allele frequency that is less than 15%. In some embodiments, the subject has a TP53 mutation allele frequency that is less than 12%. In some embodiments, the subject has a TP53 mutation allele frequency that is less than 10%. In some embodiments, the subject has a TP53 mutation allele frequency that is less than 8%. In some embodiments, the subject has a TP53 mutation allele frequency that is less than 7%. In some embodiments, the subject has a TP53 mutation allele frequency that is less than 6%. In some embodiments, the subject has a TP53 mutation allele frequency that is or less than 5%. In some embodiments, the subject does not have an activating mutation in the TP53 gene.

In some embodiments, provided herein are methods to select cancer patients for tipifarnib treatment, methods to predict the responsiveness of tipifarnib treatment in a cancer patient, and methods to increase the responsiveness to tipifarnib treatment in a cancer patient population based the mutation status of KRAS and TP53. In some embodiments, provided herein are methods of treating cancer in a subject by administering a therapeutically effective amount of tipifarnib to the subject, wherein the subject has a low KRAS mutation allele frequency and a low TP53 mutation allele frequency. In some embodiments, the subject has a TP53 mutation allele frequency that is less than 15% and a KRAS mutation allele frequency that is less than 15%. In some embodiments, the subject has a TP53 mutation allele frequency that is less than 12% and a KRAS mutation allele frequency that is less than 12%. In some embodiments, the subject has a TP53 mutation allele frequency that is less than 10% and a KRAS mutation allele frequency that is less than 10%. In some embodiments, the subject has a TP53 mutation allele frequency that is less than 8% and a KRAS mutation allele frequency that is less than 8%. In some embodiments, the subject has a TP53 mutation allele frequency that is less than 7% and a KRAS mutation allele frequency that is less than 7%. In some embodiments, the subject has a TP53 mutation allele frequency that is less than 6% and a KRAS mutation allele frequency that is less than 6%. In some embodiments, the subject has a TP53 mutation allele frequency that is less than 5% and a KRAS mutation allele frequency that is less than 5%. In some embodiment, the subject has In some embodiments, the subject does not have an activating mutation in KRAS or TP53.

As a person skill in the art would understand, the markers for patient selection for tipifarnib treatment as described herein can be used independently or in any combination. As disclosed herein, both CXCL12 and CXCR4 can drive the sensitivity of a cancer to tipifarnib treatment, and factors including, for example, IGF1R pathway activity and the mutation status of TP53 or KRAS can confer resistance to tipifarnib treatment. Accordingly, for example, provided herein are methods for selecting cancer patients for tipifarnib treatment wherein the patients have (1) either CXCL12 activation (high CXCL12 expression level or high CXCL12/CXCR4 ratio) or CXCR4 activation (high CXCR4 level, high CXCR4/CXCR2 ratio, or activating mutation in CXCR4), and (2) inactive IGF1R pathway (low IGF1 expression, low IGF2 expression, high IGFBP7 expression, and/or absence of activating mutation in PIK3CA and AKT) and/or low mutation allele frequency for TP53 and/or KRAS.

### 1.2. Cancers

Provided herein are methods of treating a cancer in a subject comprising administering a therapeutically effective amount of an FTI to the subject, based on the expression level or mutation status of certain genes as described herein. As a person skilled in the art would understand, the methods described herein can be applied to a number of different types of cancers, including solid tumors and hematological cancers. In some embodiments, the cancer is a hematological cancer. In some embodiments, the cancer is a solid tumor.

In some embodiments, the cancer is hematological cancer, and provided herein are methods of treating a hematological cancer in a subject by administering a therapeutically effective amount of an FTI based on the expression level or mutation status of certain genes as described herein. In some embodiments, the hematological cancer is a myeloid hematological cancer. The myeloid hematological cancer can be selected from the group consisting of acute myeloid leukemia (AML), myeloproliferative neoplasm (MPN), myelodysplastic syndrome (MDS), chronic myelomonocytic leukemia (CMML) and chronic myeloid leukemia (CML). In some embodiments, the hematological cancer is AML. In some embodiments, the hematological cancer is CMML. In some embodiments, the hematological cancer is MPN. In some embodiments, the hematological cancer is MDS.

In some embodiments, the hematological cancer is a lymphoid hematological cancer. The lymphoid hematological cancer can be selected from the group consisting of natural killer cell lymphoma (NK lymphoma), natural killer cell leukemia (NK leukemia), cutaneous T-Cell lymphoma (CTCL), and peripheral T-cell lymphoma (PTCL). In some embodiments, the hematological cancer is NK lymphoma. In some embodiments, the hematological cancer is NK leukemia. In some embodiments, the hematological cancer is CTCL. In some embodiments, the hematological cancer is PTCL.

In some embodiments, provided herein are methods of treating a hematological cancer (e.g. AML, PTCL, CMML) in a subject by administering a therapeutically effective amount of FTI (e.g. tipifarnib) to the subject, wherein the subject has a greater CXCL12 expression than a reference level of CXCL12 expression, and a lower IGF1 expression than a reference level of IGF1 expression. In some embodiments, the subject has an IGF1 expression that is non-detectable. In some embodiments, provided herein are methods of treating a hematological cancer (e.g. AML, PTCL, CMML) in a subject by administering a therapeutically effective amount of FTI (e.g. tipifarnib) to the subject, wherein the subject has a greater CXCL12 expression than a reference level of CXCL12 expression, and a greater IGFBP7 expression than a reference level of IGFBP7 expression.

Also disclosed herein is the finding that the IGFBP7 variant L11F (rs11573021) can indicate resistance of a hematological cancer (e.g. AML, PTCL, CMML) to an FTI (e.g. tipifarnib) treatment. Accordingly, in some embodiments, the subject selected for an FTI (e.g. tipifarnib) treatment further does not carry the IGFBP7 variant L11F (rs11573021). In some embodiments, provided herein are methods of treating a hematological cancer (e.g. AML, PTCL, CMML) in a subject by administering a therapeutically effective amount of FTI (e.g. tipifarnib) to the subject, wherein the subject has a greater CXCL12 expression than a reference level of CXCL12 expression, and wherein the subject further does not carry the IGFBP7 variant L11F (rs11573021). In some embodiments, provided herein are methods of treating a hematological cancer (e.g. AML, PTCL, CMML) in a subject by administering a therapeutically effective amount of FTI (e.g. tipifarnib) to the subject, wherein the subject has a greater CXCL12 expression than a reference level of CXCL12 expression, and wherein the subject further does not carry an activating mutation in PIK3CA. In some embodiments, provided herein are methods of treating a hematological cancer (e.g. AML, PTCL, CMML) in a subject by administering a therapeutically effective amount of FTI (e.g. tipifarnib) to the subject, wherein the subject has a greater CXCL12 expression than a reference level of CXCL12 expression, and wherein the subject further does not carry an activating mutation in AKT.

In some embodiments, provided herein are methods of treating a hematological cancer (e.g. AML, PTCL, CMML) in a subject by administering a therapeutically effective amount of FTI (e.g. tipifarnib) to the subject, wherein the subject has a greater CXCL12 expression than a reference level of CXCL12 expression, and wherein the subject has (1) a lower IGF1 expression than a reference level of IGF1 expression, or (2) a greater IGFBP7 expression than a reference level of IGFBP7 expression, wherein the subject further does not carry the IGFBP7 variant L11F (rs11573021), an activating mutation in PIK3CA, and/or activating mutation in AKT.

Accordingly, the methods provided herein for selecting subjects having a hematological cancer (e.g. AML, PTCL, CMML) for an FTI (e.g. tipifarnib) treatment further include measuring IGF2 expression in the subject. In some embodiments, provided herein are methods of treating a hematological cancer (e.g. AML, PTCL, CMML) in a subject by administering a therapeutically effective amount of FTI (e.g. tipifarnib) to the subject, wherein the subject has a greater CXCL12 expression than a reference level of CXCL12 expression, and a lower IGF2 expression than a reference level of IGF2 expression. In some embodiments, the subject has an IGF2 expression that is non-detectable. In some embodiments, provided herein are methods of treating hematological cancer in a subject by administering a therapeutically effective amount of FTI (e.g. tipifarnib) to the subject, wherein the subject has a greater CXCL12 expression than a reference level of CXCL12 expression, and a greater IGF2R expression than a reference level of IGF2R expression. In some embodiments, the subject does not have a loss of heterozygosity of IGF2. In some embodiments, the subject does not have a loss of imprinting of IGF2. In some embodiments, the subject does not have a loss of heterozygosity of IGF2 or a loss of imprinting of IGF2.

In some embodiments, methods provided herein use both IGF1 and IGF2 expression levels to select hematological cancer patients for an FTI (e.g. tipifarnib) treatment. In some embodiments, provided herein are methods of treating a hematological cancer in a subject by administering a therapeutically effective amount of FTI (e.g. tipifarnib) to the subject, wherein the subject has a greater CXCL12 expression than a reference level of CXCL12 expression, a lower IGF1 expression than a reference level of IGF1 expression, and a lower IGF2 expression than a reference level of IGF2 expression. In some embodiments, the subject has an IGF1 expression that is non-detectable. In some embodiments, the subject has an IGF2 expression that is non-detectable. In some embodiments, the subject has an IGF1 expression that is non-detectable and an IGF2 expression that is non-detectable.

The expression ratio of CXCL12 to CXCR4 also indicates the activation of the CXCL12/CXCR4 pathway, and can predict the likelihood of responsiveness of a hematological cancer (e.g. AML, PTCL, CMML) to an FTI (e.g. tipifarnib) treatment. As such, provided herein are also methods to select hematological cancer patients for an FTI treatment, methods to predict the responsiveness of an FTI treatment in a hematological cancer (e.g. AML, PTCL, CMML) patient, and methods to increase the responsiveness to an FTI treatment in a hematological cancer (e.g. AML, PTCL, CMML) patient population based the expression ratio of CXCL12 to CXCR4. In some embodiments, provided herein are methods of treating hematological cancer in a subject by administering a therapeutically effective amount of an FTI to the subject, wherein the subject has a greater expression ratio of CXCL12 to CXCR4 than a reference ratio, and an inactive IGF1R pathway. In some embodiments, the reference ratio is 1/10, 1/9, 1/8, 1/7, 1/6, 1/5, 1/4, 1/3, 1/2, 1, 2, 3, 4, 5, 6, 7, 8, 9, 10, or any numeric value between 1/10 and 10.

The subject having an inactive IGF1R pathway can have a lower IGF1 expression than a reference level of IGF1 expression, or a greater IGFBP7 expression than a reference level of IGFBP7 expression. In some embodiments, provided herein are methods of treating a hematological cancer in a subject by administering a therapeutically effective amount of an FTI (e.g. tipifarnib) to the subject, wherein the subject has a greater expression ratio of CXCL12 to CXCR4 than a reference ratio, and a lower IGF1 expression than a reference level of IGF1 expression. In some embodiments, the subject has an IGF1 expression that is non-detectable. In some embodiments, provided herein are methods of treating hematological cancer in a subject by administering a therapeutically effective amount of an FTI (e.g. tipifarnib) to the subject, wherein the subject has a greater expression ratio of CXCL12 to CXCR4 than a reference ratio, and a greater IGFBP7 expression than a reference level of IGFBP7 expression.

In some embodiments, provided herein are methods of treating hematological cancer in a subject by administering a therapeutically effective amount of FTI (e.g. tipifarnib) to the subject, wherein the subject has a greater expression ratio of CXCL12 to CXCR4 than a reference ratio, and wherein the subject further does not carry the IGFBP7 variant L11F (rs11573021). In some embodiments, provided herein are methods of treating hematological cancer in a subject by administering a therapeutically effective amount of FTI (e.g. tipifarnib) to the subject, wherein the subject has a greater expression ratio of CXCL12 to CXCR4 than a reference ratio, and wherein the subject further does not carry an activating mutation in PIK3CA. In some embodiments, provided herein are methods of treating hematological cancer in a subject by administering a therapeutically effective amount of FTI (e.g. tipifarnib) to the subject, wherein the subject has a greater expression ratio of CXCL12 to CXCR4 than a reference ratio, and wherein the subject further does not carry an activating mutation in AKT.

In some embodiments, provided herein are methods of treating hematological cancer in a subject by administering a therapeutically effective amount of FTI (e.g. tipifarnib) to the subject, wherein the subject has a greater expression ratio of CXCL12 to CXCR4 than a reference ratio, and wherein the subject has (1) a lower IGF1 expression than a reference level of IGF1 expression, or (2) a greater IGFBP7 expression than a reference level of IGFBP7 expression, wherein the subject further does not carry the IGFBP7 variant L11F (rs11573021), an activating mutation in PIK3CA, and/or activating mutation in AKT.

In some embodiments, the subjects selected for an FTI (e.g. tipifarnib) treatment based on a greater expression ratio of CXCL12 to CXCR4 than a reference ratio further has a lower IGF2 expression than a reference level of IGF2 expression. In some embodiments, the subject has an IGF2 expression that is non-detectable. In some embodiments, provided herein are methods of treating hematological cancer in a subject by administering a therapeutically effective amount of an FTI (e.g. tipifarnib) to the subject, wherein the subject has a greater expression ratio of CXCL12 to CXCR4, and a greater IGF2R expression than a reference level of IGF2R expression. In some embodiments, the subject does not have a loss of heterozygosity of IGF2. In some embodiments, the subject does not have a loss of imprinting of IGF2. In some embodiments, the subject does not have a loss of heterozygosity of IGF2 or a loss of imprinting of IGF2.

In some embodiments, the subjects selected for an FTI (e.g. tipifarnib) treatment based on a greater expression ratio of CXCL12 to CXCR4 than a reference ratio further has a lower IGF1 expression than a reference level of IGF1 expression, and a lower IGF2 expression than a reference level of IGF2 expression. In some embodiments, the subject has an IGF1 expression that is non-detectable. In some embodiments, the subject has an IGF2 expression that is non-detectable. In some embodiments, the subject has an IGF1 expression that is non-detectable and an IGF2 expression that is non-detectable.

In addition, the expression level of CXCR4 can also predict the likelihood of responsiveness of a hematological cancer (e.g. AML, PTCL, CMML) to an FTI (e.g. tipifarnib) treatment. As such, provided herein are also methods to select hematological cancer patients for an FTI (e.g. tipifarnib) treatment, methods to predict the responsiveness of an FTI (e.g. tipifarnib) treatment in a hematological cancer (e.g. AML, PTCL, CMML) patient, and methods to increase the responsiveness to an FTI (e.g. tipifarnib) treatment in a hematological cancer (e.g. AML, PTCL, CMML) patient population based the expression level of CXCR4. In some embodiments, provided herein are methods of treating hematological cancer in a subject by administering a therapeutically effective amount of an FTI (e.g. tipifarnib) to the subject, wherein the subject has a greater expression level of CXCR4 than a reference expression level of CXCR4, and an inactive IGF1R pathway. In some embodiments, provided herein are methods of treating hematological cancer in a subject by administering a therapeutically effective amount of an FTI (e.g. tipifarnib) to the subject, wherein the subject has a greater expression ratio of CXCR4 to CXCR2 than a reference ratio, and an inactive IGF1R pathway. In some embodiments, provided herein are methods of treating hematological cancer in a subject by administering a therapeutically effective amount of an FTI (e.g. tipifarnib) to the subject, wherein the subject has an activating mutation in CXCR4, and an inactive IGF1R pathway.

The subject having an inactive IGF1R pathway can have a lower IGF1 expression than a reference level of IGF1 expression. In some embodiments, provided herein are methods of treating hematological cancer in a subject by administering a therapeutically effective amount of an FTI (e.g. tipifarnib) to the subject, wherein the subject has a greater expression level of CXCR4 than a reference expression level of CXCR4, and a lower IGF1 expression than a reference level of IGF1 expression. In some embodiments, provided herein are methods of treating hematological cancer in a subject by administering a therapeutically effective amount of an FTI (e.g. tipifarnib) to the subject, wherein the subject has a greater expression ratio of CXCR4 to CXCR2 than a reference expression ratio, and a lower IGF1 expression than a reference level of IGF1 expression. In some embodiments, provided herein are methods of treating hematological cancer in a subject by administering a therapeutically effective amount of an FTI (e.g. tipifarnib) to the subject, wherein the subject has an activating mutation of CXCR4, and a lower IGF1 expression than a reference level of IGF1 expression. In some embodiments, the subject has an IGF1 expression that is non-detectable.

The subject having an inactive IGF1R pathway can be a greater IGFBP7 expression than a reference level of IGFBP7 expression. In some embodiments, provided herein are methods of treating a hematological cancer in a subject by administering a therapeutically effective amount of an FTI (e.g. tipifarnib) to the subject, wherein the subject has a greater expression level of CXCR4 than a reference expression level of CXCR4, and a greater IGFBP7 expression than a reference level of IGFBP7 expression. In some embodiments, provided herein are methods of treating hematological cancer in a subject by administering a therapeutically effective amount of an FTI (e.g. tipifarnib) to the subject, wherein the subject has a greater expression ratio of CXCR4 to CXCR2 than a reference expression ratio, and a greater IGFBP7 expression than a reference level of IGFBP7 expression. In some embodiments, provided herein are methods of treating hematological cancer in a subject by administering a therapeutically effective amount of an FTI (e.g. tipifarnib) to the subject, wherein the subject has an activating mutation of CXCR4, and a greater IGFBP7 expression than a reference level of IGFBP7 expression.

In some embodiments, the reference ratio is 1/10, 1/9, 1/8, 1/7, 1/6, 1/5, 1/4, 1/3, 1/2, 1, 2, 3, 4, 5, 6, 7, 8, 9, 10, or any numeric value between 1/10 and 10.

In some embodiments, the subjects selected for an FTI (e.g. tipifarnib) treatment based on a greater expression ratio of CXCL12 to CXCR4 than a reference ratio further has a lower IGF2 expression than a reference level of IGF2 expression. In some embodiments, the subject has an IGF2 expression that is non-detectable. In some embodiments, provided herein are methods of treating hematological cancer in a subject by administering a therapeutically effective amount of an FTI (e.g. tipifarnib) to the subject, wherein the subject has a greater expression ratio of CXCL12 to CXCR4, and a greater IGF2R expression than a reference level of IGF2R expression. In some embodiments, the subject does not have a loss of heterozygosity of IGF2. In some embodiments, the subject does not have a loss of imprinting of IGF2. In some embodiments, the subject does not have a loss of heterozygosity of IGF2 or a loss of imprinting of IGF2.

In some embodiments, the subjects selected for an FTI (e.g. tipifarnib) treatment based on a greater expression ratio of CXCL12 to CXCR4 than a reference ratio further has a lower IGF1 expression than a reference level of IGF1 expression, and a lower IGF2 expression than a reference level of IGF2 expression. In some embodiments, the subject has an IGF1 expression that is non-detectable. In some embodiments, the subject has an IGF2 expression that is non-detectable. In some embodiments, the subject has an IGF1 expression that is non-detectable and an IGF2 expression that is non-detectable.

In some embodiments, the reference ratio is 1/10, 1/9, 1/8, 1/7, 1/6, 1/5, 1/4, 1/3, 1/2, 1, 2, 3, 4, 5, 6, 7, 8, 9, 10, or any numeric value between 1/10 and 10.

Disclosed herein is also the finding that KRAS activity mediates FTI (e.g. tipifarnib) resistance in treating select a hematological cancer (e.g. AML, PTCL, CMML). As such, provided herein are methods to select hematological cancer patients for an FTI treatment, methods to predict the responsiveness of an FTI treatment in a hematological cancer patient, and methods to increase the responsiveness to an FTI treatment in a hematological cancer patient population based the mutation status of KRAS. In some embodiments, provided herein are methods of treating hematological cancer in a subject by administering a therapeutically effective amount of an FTI to the subject, wherein the subject has a low KRAS mutation allele frequency. In some embodiments, the subject has a KRAS mutation allele frequency that is less than 15%. In some embodiments, the subject has a KRAS mutation allele frequency that is less than 12%. In some embodiments, the subject has a KRAS mutation allele frequency that is less than 10%. In some embodiments, the subject has a KRAS mutation allele frequency that is less than 8%. In some embodiments, the subject has a KRAS mutation allele frequency that is less than 7%. In some embodiments, the subject has a KRAS mutation allele frequency that is less than 6%. In some embodiments, the subject has a KRAS mutation allele frequency that is or less than 5%. In some embodiments, the subject does not have an activating mutation in KRAS.

Disclosed herein is also the finding that TP53 activity mediates FTI (e.g. tipifarnib) resistance in treating select a hematological cancer (e.g. AML, PTCL, CMML). As such, provided herein are methods to select a hematological cancer patients for an FTI (e.g. tipifarnib) treatment, methods to predict the responsiveness of an FTI treatment in a hematological cancer patient, and methods to increase the responsiveness to an FTI treatment in a hematological cancer patient population based the mutation status of TP53. In some embodiments, provided herein are methods of treating hematological cancer in a subject by administering a therapeutically effective amount of an FTI to the subject, wherein the subject has a low TP53 mutation allele frequency. In some embodiments, the subject has a TP53 mutation allele frequency that is less than 15%. In some embodiments, the subject has a TP53 mutation allele frequency that is less than 12%. In some embodiments, the subject has a TP53 mutation allele frequency that is less than 10%. In some embodiments, the subject has a TP53 mutation allele frequency that is less than 8%. In some embodiments, the subject has a TP53 mutation allele frequency that is less than 7%. In some embodiments, the subject has a TP53 mutation allele frequency that is less than 6%. In some embodiments, the subject has a TP53 mutation allele frequency that is or less than 5%. In some embodiments, the subject does not have an activating mutation in the TP53 gene.

In some embodiments, provided herein are methods to select a hematological cancer patients for an FTI treatment, methods to predict the responsiveness of an FTI treatment in a hematological cancer patient, and methods to increase the responsiveness to an FTI treatment in a hematological cancer patient population based the mutation status of KRAS and TP53. In some embodiments, provided herein are methods of treating a hematological cancer (e.g. AML, PTCL, CMML) in a subject by administering a therapeutically effective amount of an FTI (e.g. tipifarnib) to the subject, wherein the subject has a low KRAS mutation allele frequency and a low TP53 mutation allele frequency. In some embodiments, the subject has a TP53 mutation allele frequency that is less than 15% and a KRAS mutation allele frequency that is less than 15%. In some embodiments, the subject has a TP53 mutation allele frequency that is less than 12% and a KRAS mutation allele frequency that is less than 12%. In some embodiments, the subject has a TP53 mutation allele frequency that is less than 10% and a KRAS mutation allele frequency that is less than 10%. In some embodiments, the subject has a TP53 mutation allele frequency that is less than 8% and a KRAS mutation allele frequency that is less than 8%. In some embodiments, the subject has a TP53 mutation allele frequency that is less than 7% and a KRAS mutation allele frequency that is less than 7%. In some embodiments, the subject has a TP53 mutation allele frequency that is less than 6% and a KRAS mutation allele frequency that is less than 6%. In some embodiments, the subject has a TP53 mutation allele frequency that is less than 5% and a KRAS mutation allele frequency that is less than 5%. In some embodiment, the subject has In some embodiments, the subject does not have an activating mutation in KRAS or TP53.

As a person skill in the art would understand, the markers for patient selection for an FTI (e.g. tipifarnib) treatment as described herein can be used independently or in any combination. As disclosed herein, both CXCL12 and CXCR4 can drive the sensitivity of a hematological cancer (e.g. AML, PTCL, CMML) to an FTI (e.g. tipifarnib) treatment, and factors including, for example, IGF1R pathway activity and the mutation status of TP53 or KRAS can confer resistance to an FTI (e.g. tipifarnib) treatment. Accordingly, for example, provided herein are methods for selecting hematological cancer (e.g. AML, PTCL, CMML) patients for an FTI (e.g. tipifarnib) treatment wherein the patients have (1) either CXCL12 activation (high CXCL12 expression level or high CXCL12/CXCR4 ratio) or CXCR4 activation (high CXCR4 level, high CXCR4/CXCR2 ratio, or activating mutation in CXCR4), and (2) inactive IGF1R pathway (low IGF1 expression, low IGF2 expression, high IGFBP7 expression, and/or absence of activating mutation in PIK3CA and AKT) and/or low mutation allele frequency for TP53 and/or KRAS.

The FTI can be any FTI, including those described herein. For example, the FTI can be tipifarnib, lonafarnib, arglabin, perrilyl alcohol, L778123, L739749, FTI-277, L744832, CP-609,754, R208176, AZD3409, or BMS-214662. In some embodiments, the FTI is tipifarnib. As such, provided herein are methods to select hematological cancer (e.g. AML, PTCL, CMML) patients for tipifarnib treatment, methods to predict the responsiveness to tipifarnib treatment in a hematological cancer patient, and methods to increase the overall responsiveness of tipifarnib treatment in a hematological cancer patient population, based on the molecular biomarkers disclosed herein, including, for example, an active CXCL12/CXCR4 pathway and an inactive IGF1R pathway.

In some embodiments, the cancer is solid tumor, and provided herein are methods of treating a solid tumor in a subject by administering a therapeutically effective amount of an FTI based on the expression level or mutation status of certain genes as described herein. The solid tumor can be pancreatic cancer, bladder cancer, breast cancer, gastric cancer colorectal cancer, head and neck cancer, head and neck squamous cell carcinoma, mesothelioma, uveal melanoma, glioblastoma, adrenocortical carcinoma, esophageal cancer, melanoma, lung adenocarcinoma, prostate cancer, lung squamous carcinoma, ovarian cancer, hepatocellular carcinoma, sarcoma, or prostate cancer. In some embodiments, the solid tumor is pancreatic cancer. In some embodiments, the cancer is squamous cell carcinoma. In some embodiments, the pancreatic cancer is pancreatic ductal adenocarcinoma (PDAC). In some embodiments, the solid tumor is bladder cancer. In some embodiments, the solid tumor is breast cancer. In some embodiments, the solid tumor is gastric cancer. In some embodiments, the solid tumor is colorectal cancer. In some embodiments, the solid tumor is head and neck cancer. In some embodiments, the solid tumor is mesothelioma. In some embodiments, the solid tumor is uveal melanoma. In some embodiments, the solid tumor is glioblastoma. In some embodiments, the solid tumor is adrenocortical carcinoma. In some embodiments, the solid tumor is adrenocortical carcinoma. In some embodiments, the solid tumor is esophageal cancer. In some embodiments, the solid tumor is melanoma. In some embodiments, the solid tumor is lung adenocarcinoma. In some embodiments, the solid tumor is prostate cancer. In some embodiments, the solid tumor is lung squamous carcinoma. In some embodiments, the solid tumor is head and neck squamous cell carcinoma. In some embodiments, the solid tumor is ovarian cancer. In some embodiments, the solid tumor is hepatocellular carcinoma. In some embodiments, the solid tumor is sarcoma.

In some embodiments, provided herein are methods of treating a solid tumor (e.g. pancreatic cancer, breast cancer) in a subject by administering a therapeutically effective amount of FTI (e.g. tipifarnib) to the subject, wherein the subject has a greater CXCL12 expression than a reference level of CXCL12 expression, and a lower IGF1 expression than a reference level of IGF1 expression. In some embodiments, the subject has an IGF1 expression that is non-detectable. In some embodiments, provided herein are methods of treating a solid tumor (e.g. pancreatic cancer, breast cancer) in a subject by administering a therapeutically effective amount of FTI (e.g. tipifarnib) to the subject, wherein the subject has a greater CXCL12 expression than a reference level of CXCL12 expression, and a greater IGFBP7 expression than a reference level of IGFBP7 expression.

Also disclosed herein is the finding that the IGFBP7 variant L11F (rs11573021) can indicate resistance of a solid tumor (e.g. pancreatic cancer, breast cancer) to an FTI (e.g. tipifarnib) treatment. Accordingly, in some embodiments, the subject selected for an FTI (e.g. tipifarnib) treatment further does not carry the IGFBP7 variant L11F (rs11573021). In some embodiments, provided herein are methods of treating a solid tumor (e.g. pancreatic cancer, breast cancer) in a subject by administering a therapeutically effective amount of FTI (e.g. tipifarnib) to the subject, wherein the subject has a greater CXCL12 expression than a reference level of CXCL12 expression, and wherein the subject further does not carry the IGFBP7 variant L11F (rs11573021). In some embodiments, provided herein are methods of treating a solid tumor (e.g. pancreatic cancer, breast cancer) in a subject by administering a therapeutically effective amount of FTI (e.g. tipifarnib) to the subject, wherein the subject has a greater CXCL12 expression than a reference level of CXCL12 expression, and wherein the subject further does not carry an activating mutation in PIK3CA. In some embodiments, provided herein are methods of treating a solid tumor (e.g. pancreatic cancer, breast cancer) in a subject by administering a therapeutically effective amount of FTI (e.g. tipifarnib) to the subject, wherein the subject has a greater CXCL12 expression than a reference level of CXCL12 expression, and wherein the subject further does not carry an activating mutation in AKT.

In some embodiments, provided herein are methods of treating a solid tumor (e.g. pancreatic cancer, breast cancer) in a subject by administering a therapeutically effective amount of FTI (e.g. tipifarnib) to the subject, wherein the subject has a greater CXCL12 expression than a reference level of CXCL12 expression, and wherein the subject has (1) a lower IGF1 expression than a reference level of IGF1 expression, or (2) a greater IGFBP7 expression than a reference level of IGFBP7 expression, wherein the subject further does not carry the IGFBP7 variant L11F (rs11573021), an activating mutation in PIK3CA, and/or activating mutation in AKT.

Accordingly, the methods provided herein for selecting subjects having a solid tumor (e.g. pancreatic cancer, breast cancer) for an FTI (e.g. tipifarnib) treatment further include measuring IGF2 expression in the subject. In some embodiments, provided herein are methods of treating a solid tumor (e.g. pancreatic cancer, breast cancer) in a subject by administering a therapeutically effective amount of FTI (e.g. tipifarnib) to the subject, wherein the subject has a greater CXCL12 expression than a reference level of CXCL12 expression, and a lower IGF2 expression than a reference level of IGF2 expression. In some embodiments, the subject has an IGF2 expression that is non-detectable. In some embodiments, provided herein are methods of treating solid tumor in a subject by administering a therapeutically effective amount of FTI (e.g. tipifarnib) to the subject, wherein the subject has a greater CXCL12 expression than a reference level of CXCL12 expression, and a greater IGF2R expression than a reference level of IGF2R expression. In some embodiments, the subject does not have a loss of heterozygosity of IGF2. In some embodiments, the subject does not have a loss of imprinting of IGF2. In some embodiments, the subject does not have a loss of heterozygosity of IGF2 or a loss of imprinting of IGF2.

In some embodiments, methods provided herein use both IGF1 and IGF2 expression levels to select solid tumor patients for an FTI (e.g. tipifarnib) treatment. In some embodiments, provided herein are methods of treating a solid tumor in a subject by administering a therapeutically effective amount of FTI (e.g. tipifarnib) to the subject, wherein the subject has a greater CXCL12 expression than a reference level of CXCL12 expression, a lower IGF1 expression than a reference level of IGF1 expression, and a lower IGF2 expression than a reference level of IGF2 expression. In some embodiments, the subject has an IGF1 expression that is non-detectable. In some embodiments, the subject has an IGF2 expression that is non-detectable. In some embodiments, the subject has an IGF1 expression that is non-detectable and an IGF2 expression that is non-detectable.

The expression ratio of CXCL12 to CXCR4 also indicates the activation of the CXCL12/CXCR4 pathway, and can predict the likelihood of responsiveness of a solid tumor (e.g. pancreatic cancer, breast cancer) to an FTI (e.g. tipifarnib) treatment. As such, provided herein are also methods to select solid tumor patients for an FTI treatment, methods to predict the responsiveness of an FTI treatment in a solid tumor (e.g. pancreatic cancer, breast cancer) patient, and methods to increase the responsiveness to an FTI treatment in a solid tumor (e.g. pancreatic cancer, breast cancer) patient population based the expression ratio of CXCL12 to CXCR4. In some embodiments, provided herein are methods of treating solid tumor in a subject by administering a therapeutically effective amount of an FTI to the subject, wherein the subject has a greater expression ratio of CXCL12 to CXCR4 than a reference ratio, and an inactive IGF1R pathway. In some embodiments, the reference ratio is 1/10, 1/9, 1/8, 1/7, 1/6, 1/5, 1/4, 1/3, 1/2, 1, 2, 3, 4, 5, 6, 7, 8, 9, 10, or any numeric value between 1/10 and 10.

The subject having an inactive IGF1R pathway can have a lower IGF1 expression than a reference level of IGF1 expression, or a greater IGFBP7 expression than a reference level of IGFBP7 expression. In some embodiments, provided herein are methods of treating a solid tumor in a subject by administering a therapeutically effective amount of an FTI (e.g. tipifarnib) to the subject, wherein the subject has a greater expression ratio of CXCL12 to CXCR4 than a reference ratio, and a lower IGF1 expression than a reference level of IGF1 expression. In some embodiments, the subject has an IGF1 expression that is non-detectable. In some embodiments, provided herein are methods of treating solid tumor in a subject by administering a therapeutically effective amount of an FTI (e.g. tipifarnib) to the subject, wherein the subject has a greater expression ratio of CXCL12 to CXCR4 than a reference ratio, and a greater IGFBP7 expression than a reference level of IGFBP7 expression.

In some embodiments, provided herein are methods of treating solid tumor in a subject by administering a therapeutically effective amount of FTI (e.g. tipifarnib) to the subject, wherein the subject has a greater expression ratio of CXCL12 to CXCR4 than a reference ratio, and wherein the subject further does not carry the IGFBP7 variant L11F (rs11573021). In some embodiments, provided herein are methods of treating solid tumor in a subject by administering a therapeutically effective amount of FTI (e.g. tipifarnib) to the subject, wherein the subject has a greater expression ratio of CXCL12 to CXCR4 than a reference ratio, and wherein the subject further does not carry an activating mutation in PIK3CA. In some embodiments, provided herein are methods of treating solid tumor in a subject by administering a therapeutically effective amount of FTI (e.g. tipifarnib) to the subject, wherein the subject has a greater expression ratio of CXCL12 to CXCR4 than a reference ratio, and wherein the subject further does not carry an activating mutation in AKT.

In some embodiments, provided herein are methods of treating solid tumor in a subject by administering a therapeutically effective amount of FTI (e.g. tipifarnib) to the subject, wherein the subject has a greater expression ratio of CXCL12 to CXCR4 than a reference ratio, and wherein the subject has (1) a lower IGF1 expression than a reference level of IGF1 expression, or (2) a greater IGFBP7 expression than a reference level of IGFBP7 expression, wherein the subject further does not carry the IGFBP7 variant L11F (rs11573021), an activating mutation in PIK3CA, and/or activating mutation in AKT.

In some embodiments, the subjects selected for an FTI (e.g. tipifarnib) treatment based on a greater expression ratio of CXCL12 to CXCR4 than a reference ratio further has a lower IGF2 expression than a reference level of IGF2 expression. In some embodiments, the subject has an IGF2 expression that is non-detectable. In some embodiments, provided herein are methods of treating solid tumor in a subject by administering a therapeutically effective amount of an FTI (e.g. tipifarnib) to the subject, wherein the subject has a greater expression ratio of CXCL12 to CXCR4, and a greater IGF2R expression than a reference level of IGF2R expression. In some embodiments, the subject does not have a loss of heterozygosity of IGF2. In some embodiments, the subject does not have a loss of imprinting of IGF2. In some embodiments, the subject does not have a loss of heterozygosity of IGF2 or a loss of imprinting of IGF2.

In some embodiments, the subjects selected for an FTI (e.g. tipifarnib) treatment based on a greater expression ratio of CXCL12 to CXCR4 than a reference ratio further has a lower IGF1 expression than a reference level of IGF1 expression, and a lower IGF2 expression than a reference level of IGF2 expression. In some embodiments, the subject has an IGF1 expression that is non-detectable. In some embodiments, the subject has an IGF2 expression that is non-detectable. In some embodiments, the subject has an IGF1 expression that is non-detectable and an IGF2 expression that is non-detectable.

In addition, the expression level of CXCR4 can also predict the likelihood of responsiveness of a solid tumor (e.g. pancreatic cancer, breast cancer) to an FTI (e.g. tipifarnib) treatment. As such, provided herein are also methods to select solid tumor patients for an FTI (e.g. tipifarnib) treatment, methods to predict the responsiveness of an FTI (e.g. tipifarnib) treatment in a solid tumor (e.g. pancreatic cancer, breast cancer) patient, and methods to increase the responsiveness to an FTI (e.g. tipifarnib) treatment in a solid tumor (e.g. pancreatic cancer, breast cancer) patient population based the expression level of CXCR4. In some embodiments, provided herein are methods of treating solid tumor in a subject by administering a therapeutically effective amount of an FTI (e.g. tipifarnib) to the subject, wherein the subject has a greater expression level of CXCR4 than a reference expression level of CXCR4, and an inactive IGF1R pathway. In some embodiments, provided herein are methods of treating solid tumor in a subject by administering a therapeutically effective amount of an FTI (e.g. tipifarnib) to the subject, wherein the subject has a greater expression ratio of CXCR4 to CXCR2 than a reference ratio, and an inactive IGF1R pathway. In some embodiments, provided herein are methods of treating solid tumor in a subject by administering a therapeutically effective amount of an FTI (e.g. tipifarnib) to the subject, wherein the subject has an activating mutation in CXCR4, and an inactive IGF1R pathway.

The subject having an inactive IGF1R pathway can have a lower IGF1 expression than a reference level of IGF1 expression. In some embodiments, provided herein are methods of treating solid tumor in a subject by administering a therapeutically effective amount of an FTI (e.g. tipifarnib) to the subject, wherein the subject has a greater expression level of CXCR4 than a reference expression level of CXCR4, and a lower IGF1 expression than a reference level of IGF1 expression. In some embodiments, provided herein are methods of treating solid tumor in a subject by administering a therapeutically effective amount of an FTI (e.g. tipifarnib) to the subject, wherein the subject has a greater expression ratio of CXCR4 to CXCR2 than a reference expression ratio, and a lower IGF1 expression than a reference level of IGF1 expression. In some embodiments, provided herein are methods of treating solid tumor in a subject by administering a therapeutically effective amount of an FTI (e.g. tipifarnib) to the subject, wherein the subject has an activating mutation of CXCR4, and a lower IGF1 expression than a reference level of IGF1 expression. In some embodiments, the subject has an IGF1 expression that is non-detectable.

The subject having an inactive IGF1R pathway can be a greater IGFBP7 expression than a reference level of IGFBP7 expression. In some embodiments, provided herein are methods of treating a solid tumor in a subject by administering a therapeutically effective amount of an FTI (e.g. tipifarnib) to the subject, wherein the subject has a greater expression level of CXCR4 than a reference expression level of CXCR4, and a greater IGFBP7 expression than a reference level of IGFBP7 expression. In some embodiments, provided herein are methods of treating solid tumor in a subject by administering a therapeutically effective amount of an FTI (e.g. tipifarnib) to the subject, wherein the subject has a greater expression ratio of CXCR4 to CXCR2 than a reference expression ratio, and a greater IGFBP7 expression than a reference level of IGFBP7 expression. In some embodiments, provided herein are methods of treating solid tumor in a subject by administering a therapeutically effective amount of an FTI (e.g. tipifarnib) to the subject, wherein the subject has an activating mutation of CXCR4, and a greater IGFBP7 expression than a reference level of IGFBP7 expression.

In some embodiments, the subjects selected for an FTI (e.g. tipifarnib) treatment based on a greater expression ratio of CXCL12 to CXCR4 than a reference ratio further has a lower IGF2 expression than a reference level of IGF2 expression. In some embodiments, the subject has an IGF2 expression that is non-detectable. In some embodiments, provided herein are methods of treating solid tumor in a subject by administering a therapeutically effective amount of an FTI (e.g. tipifarnib) to the subject, wherein the subject has a greater expression ratio of CXCL12 to CXCR4, and a greater IGF2R expression than a reference level of IGF2R expression. In some embodiments, the subject does not have a loss of heterozygosity of IGF2. In some embodiments, the subject does not have a loss of imprinting of IGF2. In some embodiments, the subject does not have a loss of heterozygosity of IGF2 or a loss of imprinting of IGF2.

In some embodiments, the subjects selected for an FTI (e.g. tipifarnib) treatment based on a greater expression ratio of CXCL12 to CXCR4 than a reference ratio further has a lower IGF1 expression than a reference level of IGF1 expression, and a lower IGF2 expression than a reference level of IGF2 expression. In some embodiments, the subject has an IGF1 expression that is non-detectable. In some embodiments, the subject has an IGF2 expression that is non-detectable. In some embodiments, the subject has an IGF1 expression that is non-detectable and an IGF2 expression that is non-detectable.

In some embodiments, the reference ratio is 1/10, 1/9, 1/8, 1/7, 1/6, 1/5, 1/4, 1/3, 1/2, 1, 2, 3, 4, 5, 6, 7, 8, 9, 10, or any numeric value between 1/10 and 10.

Disclosed herein is also the finding that KRAS activity mediates FTI (e.g. tipifarnib) resistance in treating select a solid tumor (e.g. pancreatic cancer, breast cancer). As such, provided herein are methods to select solid tumor patients for an FTI treatment, methods to predict the responsiveness of an FTI treatment in a solid tumor patient, and methods to increase the responsiveness to an FTI treatment in a solid tumor patient population based the mutation status of KRAS. In some embodiments, provided herein are methods of treating solid tumor in a subject by administering a therapeutically effective amount of an FTI to the subject, wherein the subject has a low KRAS mutation allele frequency (Variant Allele Frequency, VAF). In some embodiments, the subject has a KRAS mutation allele frequency that is less than 15%. In some embodiments, the subject has a KRAS mutation allele frequency that is less than 12%. In some embodiments, the subject has a KRAS mutation allele frequency that is less than 10%. In some embodiments, the subject has a KRAS mutation allele frequency that is less than 8%. In some embodiments, the subject has a KRAS mutation allele frequency that is less than 7%. In some embodiments, the subject has a KRAS mutation allele frequency that is less than 6%. In some embodiments, the subject has a KRAS mutation allele frequency that is or less than 5%. In some embodiments, the subject does not have an activating mutation in KRAS. In some embodiments, if several samples are available, KRAS testing should be performed in the most recently obtained tumor sample.

In some embodiments, provided herein are methods of treating a solid tumor (e.g. pancreatic cancer, breast cancer) in a subject by administering a therapeutically effective amount of FTI (e.g. tipifarnib) to the subject, wherein the subject has a greater CXCL12 expression than a reference level of CXCL12 expression, and wherein the KRAS mutation allele frequency (Variant Allele Frequency, VAF) is or is less than 5%.

Disclosed herein is also the finding that TP53 activity mediates FTI (e.g. tipifarnib) resistance in treating select a solid tumor (e.g. pancreatic cancer, breast cancer). As such, provided herein are methods to select a solid tumor patients for an FTI (e.g. tipifarnib) treatment, methods to predict the responsiveness of an FTI treatment in a solid tumor patient, and methods to increase the responsiveness to an FTI treatment in a solid tumor patient population based the mutation status of TP53. In some embodiments, provided herein are methods of treating solid tumor in a subject by administering a therapeutically effective amount of an FTI to the subject, wherein the subject has a low TP53 mutation allele frequency. In some embodiments, the subject has a TP53 mutation allele frequency that is less than 15%. In some embodiments, the subject has a TP53 mutation allele frequency that is less than 12%. In some embodiments, the subject has a TP53 mutation allele frequency that is less than 10%. In some embodiments, the subject has a TP53 mutation allele frequency that is less than 8%. In some embodiments, the subject has a TP53 mutation allele frequency that is less than 7%. In some embodiments, the subject has a TP53 mutation allele frequency that is less than 6%. In some embodiments, the subject has a TP53 mutation allele frequency that is or less than 5%. In some embodiments, the subject does not have an activating mutation in the TP53 gene.

In some embodiments, provided herein are methods to select solid tumor patients for an FTI treatment, methods to predict the responsiveness of an FTI treatment in a solid tumor patient, and methods to increase the responsiveness to an FTI treatment in a solid tumor patient population based the mutation status of KRAS and TP53. In some embodiments, provided herein are methods of treating a solid tumor (e.g. pancreatic cancer, breast cancer) in a subject by administering a therapeutically effective amount of an FTI (e.g. tipifarnib) to the subject, wherein the subject has a low KRAS mutation allele frequency and a low TP53 mutation allele frequency. In some embodiments, the subject has a TP53 mutation allele frequency that is less than 15% and a KRAS mutation allele frequency that is less than 15%. In some embodiments, the subject has a TP53 mutation allele frequency that is less than 12% and a KRAS mutation allele frequency that is less than 12%. In some embodiments, the subject has a TP53 mutation allele frequency that is less than 10% and a KRAS mutation allele frequency that is less than 10%. In some embodiments, the subject has a TP53 mutation allele frequency that is less than 8% and a KRAS mutation allele frequency that is less than 8%. In some embodiments, the subject has a TP53 mutation allele frequency that is less than 7% and a KRAS mutation allele frequency that is less than 7%. In some embodiments, the subject has a TP53 mutation allele frequency that is less than 6% and a KRAS mutation allele frequency that is less than 6%. In some embodiments, the subject has a TP53 mutation allele frequency that is less than 5% and a KRAS mutation allele frequency that is less than 5%. In some embodiment, the subject has In some embodiments, the subject does not have an activating mutation in KRAS or TP53.

As a person skill in the art would understand, the markers for patient selection for an FTI (e.g. tipifarnib) treatment as described herein can be used independently or in any combination. As disclosed herein, both CXCL12 and CXCR4 can drive the sensitivity of a solid tumor (e.g. pancreatic cancer, breast cancer) to an FTI (e.g. tipifarnib) treatment, and factors including, for example, IGF1R pathway activity and the mutation status of TP53 or KRAS can confer resistance to an FTI (e.g. tipifarnib) treatment. Accordingly, for example, provided herein are methods for selecting solid tumor (e.g. pancreatic cancer, breast cancer) patients for an FTI (e.g. tipifarnib) treatment wherein the patients have (1) either CXCL12 activation (high CXCL12 expression level or high CXCL12/CXCR4 ratio) or CXCR4 activation (high CXCR4 level, high CXCR4/CXCR2 ratio, or activating mutation in CXCR4), and (2) inactive IGF1R pathway (low IGF1 expression, low IGF2 expression, high IGFBP7 expression, and/or absence of activating mutation in PIK3CA and AKT) and/or low mutation allele frequency for TP53 and/or KRAS.

The FTI can be any FTI, including those described herein. For example, the FTI can be tipifarnib, lonafarnib, arglabin, perrilyl alcohol, L778123, L739749, FTI-277, L744832, CP-609,754, R208176, AZD3409, or BMS-214662. In some embodiments, the FTI is tipifarnib. As such, provided herein are methods to select solid tumor (e.g. pancreatic cancer, breast cancer) patients for tipifarnib treatment, methods to predict the responsiveness to tipifarnib treatment in a solid tumor patient, and methods to increase the overall responsiveness of tipifarnib treatment in a solid tumor patient population, based on the molecular biomarkers disclosed herein, including, for example, an active CXCL12/CXCR4 pathway and an inactive IGF1R pathway.

As a person of ordinary skill in the art would understand, the mechanistic basis disclosed herein for selecting cancer patients for FTI treatment is generally applicable to all cancer types, and is therefore not limited to a particular cancer type or a particular FTI. Accordingly, although specific embodiments are described herein for exemplary purpose, all permutations and combinations of different molecule signatures (expression level, expression ratio, and mutation status), cancer types, and FTIs as described herein are expressly contemplated.

As described above, provided herein are methods of selecting breast cancer patients for an FTI treatment (e.g. tipifarnib), methods of determining the responsiveness of a breast cancer patient to an FTI treatment (e.g. tipifarnib), and methods of increasing the overall responsiveness of a breast cancer patient population to an FTI treatment (e.g. tipifarnib) based on selected molecular signatures, including, for example, the activities of CXCL12/CXCR4 and IGF1R pathways. As is known in the art, breast cancer can be divided into different subtypes based on the expression of estrogen receptor (ER) or progesterone receptor (PR), and the positivity of ER and/or indicates the likelihood of responsiveness to different treatment.

Disclosed herein is also the finding that PR positivity enriches for sensitivity to an FTI treatment (e.g. tipifarnib), and that PR positive and ER negative tumors are very sensitive to an FTI treatment. Accordingly, methods provided herein further include selecting breast cancer patients for an FTI treatment based on the expression of these receptors. In some embodiments, provided herein are methods of treating a breast cancer in a subject by administering a therapeutically effective amount of FTI (e.g. tipifarnib) to the subject, wherein the subject is PR positive. In some embodiments, provided herein are methods of treating a breast cancer in a subject by administering a therapeutically effective amount of FTI (e.g. tipifarnib) to the subject, wherein the subject is ER negative. In some embodiments, provided herein are methods of treating a breast cancer in a subject by administering a therapeutically effective amount of FTI (e.g. tipifarnib) to the subject, wherein the subject is both PR positive and ER negative.

As described above, provided herein are methods of selecting pancreatic cancer patients for an FTI treatment (e.g. tipifarnib), methods of determining the responsiveness of a pancreatic cancer patient to an FTI treatment, and methods of increasing the overall responsiveness of a pancreatic cancer patient population to an FTI treatment based on selected molecular signatures, including, for example, the activities of CXCL12/CXCR4 and IGF1R pathways. Disclosed herein is also the finding that these molecule signatures are also related to the clinical presentation of the pancreatic cancer, and that various clinical presentations can serve as basis for predicating responsiveness of a pancreatic cancer patient to an FTI treatment, and for selecting pancreatic cancer patients to an FTI treatment to increase the overall responsive rate. Specifically, disclosed herein is the finding that the activation of CXCL12/CXCR4 pathway by CXCL12 overexpression is associated with nodal metastases, and/or is characterized by absence of abdominal pain, the phenomenon of analgesia due to Schwan cell expressing CXCR7 and migration to CXCL12 producing tumors. As such, nodal metastases and absence of abdominal pain in a pancreatic cancer patient indicates that the pancreatic cancer patient has high expression of CXCL12, and is likely responsive to an FTI treatment. Accordingly, in some embodiments, provided herein are methods of treating a pancreatic cancer in a subject by administering a therapeutically effective amount of FTI (e.g. tipifarnib) to the subject, wherein the subject has nodal metastases, and/or is characterized by absence of abdominal pain. In some embodiments, methods provided herein further include determining whether a subject having pancreatic cancer has abdominal pain, and administering a therapeutically effective amount of FTI (e.g. tipifarnib) to the subject if the subject has no abdominal pain, and if the subject has nodal metastases. In some embodiments, methods provided herein further include administering a therapeutically effective amount of FTI (e.g. tipifarnib) to a subject who has previously been treated with folfirinox.

Disclosed herein is also the finding that the activation of CXCL12/CXCR4 pathway by CXCR4 overexpression is associated with liver metastases, and are characterized by healthy liver function. As such, liver metastases and physiological parameters indicating healthy liver function in a pancreatic cancer patient indicates that the pancreatic cancer patient has high expression of CXCR4, and is likely responsive to an FTI (e.g. tipifarnib) treatment.

Accordingly, in some embodiments, provided herein are methods of treating a pancreatic cancer in a subject by administering a therapeutically effective amount of FTI (e.g. tipifarnib) to the subject, wherein the subject has (i) liver metastases or nodal metastasis; and (ii) (1) an aspartate transaminase (AST) level, (2) an alanine transaminase (ALT) level, (3) an alkaline phosphatase (ALP), and/or (4) a total bilirubin level that is within normal range, or lower than the respective upper normal limit (UNL). In some embodiments, methods provided herein further include determining the AST level, ALT level, ALP level, total bilirubin level, or any combination thereof, in a subject to determine whether the subject is likely responsive to an FTI (e.g. tipifarnib) treatment.

In some embodiments, provided herein are methods of treating a pancreatic cancer in a subject by administering a therapeutically effective amount of FTI (e.g. tipifarnib) to the subject, wherein the subject has liver metastases and an AST level that is within normal range, or lower than the UNL of ASL. The methods can further include determining the AST level in the subject. In some embodiments, provided herein are methods of treating a pancreatic cancer in a subject by administering a therapeutically effective amount of FTI (e.g. tipifarnib) to the subject, wherein the subject has liver metastases and an ALT level that is within normal range, or lower than the UNL of ALT. The methods can further include determining the ALT level in the subject. In some embodiments, provided herein are methods of treating a pancreatic cancer in a subject by administering a therapeutically effective amount of FTI (e.g. tipifarnib) to the subject, wherein the subject has liver metastases and an ALP level that is within normal range, or lower than the UNL of ALP. The methods can further include determining the ALP level in the subject. In some embodiments, provided herein are methods of treating a pancreatic cancer in a subject by administering a therapeutically effective amount of FTI (e.g. tipifarnib) to the subject, wherein the subject has liver metastases and a total bilirubin level that is within normal range, or lower than the UNL of the total bilirubin level. The methods can further include determining the total bilirubin level in the subject. Disclosed herein are also various permutations and combinations of the methods described above.

As used herein in connection with a physiologic measurement, the “normal range” is the range of values for the measurement in healthy population. In otherwise, a measurement falling within the normal indicates adequate physiological function. The UNL is the upper limit of the normal range. In some embodiments, the UNL of AST can be 40 U/L. In some embodiments, the UNL of ALT can be 55 U/L. In some embodiments, the UNL for ALP can be 120 U/L. In some embodiments, the UNL of total bilirubin level can be 1.0 mg/dL. As known in the art, the normal range and UNL of a specific parameter can vary among different demographic populations. Methods of determining the clinical presentation and measuring the clinical parameters described herein are well known in the art.

### 1.3. Assays

As described herein, the expression level of a gene (e.g. CXCL12, IGF1, IGFBP7, IGF2, IGF2R, CXCR4, or CXCR2) can refer to the protein level or the mRNA level of the gene. In some embodiments, the expression level of a gene refers to the mRNA level of the gene. In some embodiments, methods provided herein further include determining the mRNA level of the gene. In some embodiments, the expression level of a gene refers to the protein level of the gene. In some embodiments, methods provided herein further include determining the protein level of the gene.

In some embodiments, the expression level of a gene can refer to the mRNA level of the gene. As such, the CXCL12 expression level can refer to the mRNA level of CXCL12 in a sample. The IGF1 expression level can refer to the mRNA level of IGF1 in a sample. The IGFBP7 expression level can refer to the mRNA level of IGFBP7 in a sample. The IGF2 expression level can refer to the mRNA level of IGF2 in a sample. The IGF2R expression level can refer to the mRNA level of IGF2R in a sample. The CXCR4 expression level can refer to the mRNA level of CXCR4 in a sample. The CXCR2 expression level can refer to the mRNA level of CXCR2 in a sample.

In some embodiments, provided herein are methods of treating a cancer in a subject by administering a therapeutically effective amount of an FTI based on the mRNA level or mutation status of certain genes as described herein. In some embodiments, provided herein are methods of treating a cancer in a subject by administering a therapeutically effective amount of FTI (e.g. tipifarnib) to the subject, wherein the subject has a greater CXCL12 mRNA level than a reference level of CXCL12 mRNA, and a lower IGF1 mRNA level than a reference mRNA level of IGF1. In some embodiments, the subject has an IGF1 mRNA level that is non-detectable. In some embodiments, provided herein are methods of treating a cancer in a subject by administering a therapeutically effective amount of FTI (e.g. tipifarnib) to the subject, wherein the subject has a greater CXCL12 mRNA level than a reference mRNA level of CXCL12, and a greater IGFBP7 mRNA level than a reference mRNA level of IGFBP7.

In some embodiments, the subject selected for an FTI (e.g. tipifarnib) treatment further does not carry the IGFBP7 variant L11F (rs11573021). In some embodiments, provided herein are methods of treating a cancer in a subject by administering a therapeutically effective amount of FTI (e.g. tipifarnib) to the subject, wherein the subject has a greater CXCL12 mRNA level than a reference mRNA level of CXCL12 expression, and wherein the subject further does not carry the IGFBP7 variant L11F (rs11573021). In some embodiments, provided herein are methods of treating a cancer in a subject by administering a therapeutically effective amount of FTI (e.g. tipifarnib) to the subject, wherein the subject has a greater CXCL12 mRNA level than a reference mRNA level of CXCL12, and wherein the subject further does not carry an activating mutation in PIK3CA. In some embodiments, provided herein are methods of treating a cancer in a subject by administering a therapeutically effective amount of FTI (e.g. tipifarnib) to the subject, wherein the subject has a greater CXCL12 mRNA level than a reference mRNA level of CXCL12, and wherein the subject further does not carry an activating mutation in AKT.

In some embodiments, provided herein are methods of treating a cancer in a subject by administering a therapeutically effective amount of FTI (e.g. tipifarnib) to the subject, wherein the subject has a greater CXCL12 mRNA level than a reference mRNA level of CXCL12, and wherein the subject has (1) a lower IGF1 mRNA level than a reference mRNA level of IGF1, or (2) a greater IGFBP7 mRNA level than a reference mRNA level of IGFBP7, wherein the subject further does not carry the IGFBP7 variant L11F (rs11573021), an activating mutation in PIK3CA, and/or activating mutation in AKT.

Accordingly, the methods provided herein for selecting subjects having a cancer for an FTI (e.g. tipifarnib) treatment further include measuring IGF2 mRNA level in the subject. In some embodiments, provided herein are methods of treating a cancer in a subject by administering a therapeutically effective amount of FTI (e.g. tipifarnib) to the subject, wherein the subject has a greater CXCL12 mRNA level than a reference mRNA level of CXCL12, and a lower IGF2 mRNA level than a reference mRNA level of IGF2. In some embodiments, the subject has an IGF2 mRNA level that is non-detectable. In some embodiments, provided herein are methods of treating cancer in a subject by administering a therapeutically effective amount of FTI (e.g. tipifarnib) to the subject, wherein the subject has a greater CXCL12 mRNA level than a reference mRNA level of CXCL12, and a greater IGF2R mRNA level than a reference mRNA level of IGF2R. In some embodiments, the subject does not have a loss of heterozygosity of IGF2. In some embodiments, the subject does not have a loss of imprinting of IGF2. In some embodiments, the subject does not have a loss of heterozygosity of IGF2 or a loss of imprinting of IGF2.

In some embodiments, methods provided herein use both IGF1 and IGF2 mRNA levels to select cancer patients for an FTI (e.g. tipifarnib) treatment. In some embodiments, provided herein are methods of treating a cancer in a subject by administering a therapeutically effective amount of FTI (e.g. tipifarnib) to the subject, wherein the subject has a greater CXCL12 mRNA level than a reference mRNA level of CXCL12, a lower IGF1 mRNA level than a reference mRNA level of IGF1, and a lower IGF2 mRNA level than a reference mRNA level of IGF2. In some embodiments, the subject has an IGF1 mRNA level that is non-detectable. In some embodiments, the subject has an IGF2 mRNA level that is non-detectable. In some embodiments, the subject has an IGF1 mRNA level that is non-detectable and an IGF2 mRNA level that is non-detectable.

The mRNA ratio of CXCL12 to CXCR4 also indicates the activation of the CXCL12/CXCR4 pathway, and can predict the likelihood of responsiveness of a cancer to an FTI (e.g. tipifarnib) treatment. As such, provided herein are also methods to select cancer patients for an FTI treatment, methods to predict the responsiveness of an FTI treatment in a cancer patient, and methods to increase the responsiveness to an FTI treatment in a cancer patient population based the mRNA ratio of CXCL12 to CXCR4. In some embodiments, provided herein are methods of treating cancer in a subject by administering a therapeutically effective amount of an FTI to the subject, wherein the subject has a greater ratio of CXCL12 mRNA level to CXCR4 mRNA level than a reference ratio, and an inactive IGF1R pathway.

The subject having an inactive IGF1R pathway can have a lower IGF1 mRNA level than a reference mRNA level of IGF1, or a greater IGFBP7 mRNA level than a reference mRNA level of IGFBP7. In some embodiments, provided herein are methods of treating a cancer in a subject by administering a therapeutically effective amount of an FTI (e.g. tipifarnib) to the subject, wherein the subject has a greater ratio of CXCL12 mRNA level to CXCR4 mRNA level than a reference ratio, and a lower IGF1 mRNA level than a reference mRNA level of IGF1. In some embodiments, the subject has an IGF1 mRNA level that is non-detectable. In some embodiments, provided herein are methods of treating cancer in a subject by administering a therapeutically effective amount of an FTI (e.g. tipifarnib) to the subject, wherein the subject has a greater ratio of CXCL12 mRNA level to CXCR4 mRNA level than a reference ratio, and a greater IGFBP7 mRNA level than a reference mRNA level of IGFBP7.

In some embodiments, provided herein are methods of treating cancer in a subject by administering a therapeutically effective amount of FTI (e.g. tipifarnib) to the subject, wherein the subject has a greater ratio of CXCL12 mRNA level to CXCR4 mRNA level than a reference ratio, and wherein the subject further does not carry the IGFBP7 variant L11F (rs11573021). In some embodiments, provided herein are methods of treating cancer in a subject by administering a therapeutically effective amount of FTI (e.g. tipifarnib) to the subject, wherein the subject has a greater ratio of CXCL12 mRNA level to CXCR4 mRNA level than a reference ratio, and wherein the subject further does not carry an activating mutation in PIK3CA. In some embodiments, provided herein are methods of treating cancer in a subject by administering a therapeutically effective amount of FTI (e.g. tipifarnib) to the subject, wherein the subject has a greater ratio of CXCL12 mRNA level to CXCR4 mRNA level than a reference ratio, and wherein the subject further does not carry an activating mutation in AKT.

In some embodiments, provided herein are methods of treating cancer in a subject by administering a therapeutically effective amount of FTI (e.g. tipifarnib) to the subject, wherein the subject has a greater ratio of CXCL12 mRNA level to CXCR4 mRNA level than a reference ratio, and wherein the subject has (1) a lower IGF1 mRNA level than a reference mRNA level of IGF1, or (2) a greater IGFBP7 mRNA level than a reference mRNA level of IGFBP7, wherein the subject further does not carry the IGFBP7 variant L11F (rs11573021), an activating mutation in PIK3CA, and/or activating mutation in AKT.

In some embodiments, the subjects selected for an FTI (e.g. tipifarnib) treatment based on a greater ratio of CXCL12 mRNA level to CXCR4 mRNA level than a reference ratio further has a lower IGF2 mRNA level than a reference mRNA level of IGF2. In some embodiments, the subject has an IGF2 mRNA level that is non-detectable. In some embodiments, provided herein are methods of treating cancer in a subject by administering a therapeutically effective amount of an FTI (e.g. tipifarnib) to the subject, wherein the subject has a greater ratio of CXCL12 mRNA level to CXCR4 mRNA level, and a greater IGF2R mRNA level than a reference mRNA level of IGF2R. In some embodiments, the subject does not have a loss of heterozygosity of IGF2. In some embodiments, the subject does not have a loss of imprinting of IGF2. In some embodiments, the subject does not have a loss of heterozygosity of IGF2 or a loss of imprinting of IGF2.

In some embodiments, the subjects selected for an FTI (e.g. tipifarnib) treatment based on a greater ratio of CXCL12 mRNA level to CXCR4 mRNA level than a reference ratio further has a lower IGF1 mRNA level than a reference mRNA level of IGF1, and a lower IGF2 mRNA level than a reference mRNA level of IGF2. In some embodiments, the subject has an IGF1 mRNA level that is non-detectable. In some embodiments, the subject has an IGF2 mRNA level that is non-detectable. In some embodiments, the subject has an IGF1 mRNA level that is non-detectable and an IGF2 mRNA level that is non-detectable.

In addition, the mRNA level of CXCR4 can also predict the likelihood of responsiveness of a cancer to an FTI (e.g. tipifarnib) treatment. As such, provided herein are also methods to select cancer patients for an FTI (e.g. tipifarnib) treatment, methods to predict the responsiveness of an FTI (e.g. tipifarnib) treatment in a cancer patient, and methods to increase the responsiveness to an FTI (e.g. tipifarnib) treatment in a cancer patient population based the mRNA level of CXCR4. In some embodiments, provided herein are methods of treating cancer in a subject by administering a therapeutically effective amount of an FTI (e.g. tipifarnib) to the subject, wherein the subject has a greater mRNA level of CXCR4 than a reference mRNA level of CXCR4, and an inactive IGF1R pathway. In some embodiments, provided herein are methods of treating cancer in a subject by administering a therapeutically effective amount of an FTI (e.g. tipifarnib) to the subject, wherein the subject has a greater ratio of CXCR4 mRNA level to CXCR2 mRNA level than a reference ratio, and an inactive IGF1R pathway. In some embodiments, provided herein are methods of treating cancer in a subject by administering a therapeutically effective amount of an FTI (e.g. tipifarnib) to the subject, wherein the subject has an activating mutation in CXCR4, and an inactive IGF1R pathway.

The subject having an inactive IGF1R pathway can have a lower IGF1 mRNA level than a reference mRNA level of IGF1. In some embodiments, provided herein are methods of treating cancer in a subject by administering a therapeutically effective amount of an FTI (e.g. tipifarnib) to the subject, wherein the subject has a greater mRNA level of CXCR4 than a reference mRNA level of CXCR4, and a lower IGF1 mRNA level than a reference mRNA level of IGF1. In some embodiments, provided herein are methods of treating cancer in a subject by administering a therapeutically effective amount of an FTI (e.g. tipifarnib) to the subject, wherein the subject has a greater ratio of CXCR4 mRNA level to CXCR2 mRNA level than a reference ratio, and a lower IGF1 mRNA level than a reference mRNA level of IGF1. In some embodiments, provided herein are methods of treating cancer in a subject by administering a therapeutically effective amount of an FTI (e.g. tipifarnib) to the subject, wherein the subject has an activating mutation of CXCR4, and a lower IGF1 mRNA level than a reference mRNA level of IGF1. In some embodiments, the subject has an IGF1 mRNA level that is non-detectable.

The subject having an inactive IGF1R pathway can be a greater IGFBP7 mRNA level than a mRNA level of IGFBP7. In some embodiments, provided herein are methods of treating a cancer in a subject by administering a therapeutically effective amount of an FTI (e.g. tipifarnib) to the subject, wherein the subject has a greater mRNA level of CXCR4 than a reference mRNA level of CXCR4, and a greater IGFBP7 mRNA level than a reference mRNA level of IGFBP7. In some embodiments, provided herein are methods of treating cancer in a subject by administering a therapeutically effective amount of an FTI (e.g. tipifarnib) to the subject, wherein the subject has a greater ratio of CXCR4 mRNA level to CXCR2 mRNA level than a reference ratio, and a greater IGFBP7 mRNA level than a reference mRNA level of IGFBP7. In some embodiments, provided herein are methods of treating cancer in a subject by administering a therapeutically effective amount of an FTI (e.g. tipifarnib) to the subject, wherein the subject has an activating mutation of CXCR4, and a greater IGFBP7 mRNA level than a reference mRNA level of IGFBP7.

In some embodiments, the subjects selected for an FTI (e.g. tipifarnib) treatment based on a greater ratio of CXCL12 mRNA level to CXCR4 mRNA level than a reference ratio further has a lower IGF2 mRNA level than a reference mRNA level of IGF2. In some embodiments, the subject has an IGF2 mRNA level that is non-detectable. In some embodiments, provided herein are methods of treating cancer in a subject by administering a therapeutically effective amount of an FTI (e.g. tipifarnib) to the subject, wherein the subject has a greater ratio of CXCL12 mRNA level to CXCR4 mRNA level, and a greater IGF2R mRNA level than a reference mRNA level of IGF2R. In some embodiments, the subject does not have a loss of heterozygosity of IGF2. In some embodiments, the subject does not have a loss of imprinting of IGF2. In some embodiments, the subject does not have a loss of heterozygosity of IGF2 or a loss of imprinting of IGF2.

In some embodiments, the subjects selected for an FTI (e.g. tipifarnib) treatment based on a greater ratio of CXCL12 mRNA level to CXCR4 mRNA level than a reference ratio further has a lower IGF1 mRNA level than a reference mRNA level of IGF1, and a lower IGF2 mRNA level than a reference mRNA level of IGF2. In some embodiments, the subject has an IGF1 mRNA level that is non-detectable. In some embodiments, the subject has an IGF2 mRNA level that is non-detectable. In some embodiments, the subject has an IGF1 mRNA level that is non-detectable and an IGF2 mRNA level that is non-detectable.

In some embodiments, provided herein are methods for selecting cancer patients for an FTI (e.g. tipifarnib) treatment wherein the patients have (1) either CXCL12 activation (high CXCL12 mRNA level or high ratio of CXCL12 mRNA level/CXCR4 mRNA level) or CXCR4 activation (high CXCR4 mRNA level, high ratio of CXCR4 mRNA level/CXCR2 mRNA level, or activating mutation in CXCR4), and (2) inactive IGF1R pathway (low IGF1 mRNA level, low IGF2 mRNA level, high IGFBP7 mRNA level, and/or absence of activating mutation in PIK3CA and AKT) and/or low mutation allele frequency for TP53 and/or KRAS.

The FTI can be any FTI, including those described herein. For example, the FTI can be tipifarnib, lonafarnib, arglabin, perrilyl alcohol, L778123, L739749, FTI-277, L744832, CP-609,754, R208176, AZD3409, or BMS-214662. In some embodiments, the FTI is tipifarnib. As such, provided herein are methods to select cancer patients for tipifarnib treatment, methods to predict the responsiveness to tipifarnib treatment in a cancer patient, and methods to increase the overall responsiveness of tipifarnib treatment in a cancer patient population, based on the mRNA levels of molecular biomarkers disclosed herein, including, for example, the mRNA levels of CXCL12, IGF1, IGFBP7, IGF2, IGF2R, CXCR4, CXCR2, or any combination thereof.

In some embodiments, the reference mRNA level of a gene is the median mRNA level of the gene in a population of healthy subjects. In some embodiments, the reference mRNA level of a gene is the median mRNA level of the gene in a population of subjects having a particular cancer. For example, the reference mRNA level of a gene in pancreatic cancer patients is the median mRNA level of the gene in a population of subjects having pancreatic cancer. For another example, the reference mRNA level of a gene in AML patients is the median mRNA level of the gene in a population of subjects having AML. In some embodiments, the reference mRNA level of a gene is the cutoff value determined by statistical analysis by a person of ordinary skill in the art.

In some embodiments, methods provided herein include determining the mRNA level of a gene. Methods to determine the mRNA level of a gene in a sample are well known in the art. For example, in some embodiments, the mRNA level can be determined by Polymerase Chain Reaction (PCR), qPCR, qRT-PCR, RNA-seq, microarray analysis, SAGE, MassARRAY technique, next-generation sequencing, or FISH.

Exemplary methods of detecting or quantitating mRNA levels include but are not limited to PCR-based methods, northern blots, ribonuclease protection assays, and the like. The mRNA sequence can be used to prepare a probe that is at least partially complementary. The probe can then be used to detect the mRNA sequence in a sample, using any suitable assay, such as PCR-based methods, Northern blotting, a dipstick assay, and the like.

The commonly used methods known in the art for the quantification of mRNA expression in a sample include northern blotting and in situ hybridization (Parker &Barnes, Methods in Molecular Biology 106:247-283 (1999)); RNAse protection assays (Hod, Biotechniques 13:852-854 (1992)); and polymerase chain reaction (PCR) (Weis et ah, Trends in Genetics 8:263-264 (1992)). Alternatively, antibodies may be employed that can recognize specific duplexes, including DNA duplexes, RNA duplexes, and DNA-RNA hybrid duplexes or DNA-protein duplexes. Representative methods for sequencing-based gene expression analysis include Serial Analysis of Gene Expression (SAGE), and gene expression analysis by massively parallel signature sequencing (MPSS).

A sensitive and flexible quantitative method is PCR. Examples of PCR methods can be found in the literature. Examples of PCR assays can be found in U.S. Pat. No. 6,927,024, which is incorporated by reference herein in its entirety. Examples of RT-PCR methods can be found in U.S. Pat. No. 7,122,799, which is incorporated by reference herein in its entirety. A method of fluorescent in situ PCR is described in U.S. Pat. No. 7,186,507, which is incorporated by reference herein in its entirety.

It is noted, however, that other nucleic acid amplification protocols (i.e., other than PCR) may also be used in the nucleic acid analytical methods described herein. For example, suitable amplification methods include ligase chain reaction (see, e.g., Wu & Wallace, Genomics 4:560-569, 1988); strand displacement assay (see, e.g., Walker et al., Proc. Natl. Acad. Sci. USA 89:392-396, 1992; U.S. Pat. No. 5,455,166); and several transcription-based amplification systems, including the methods described in U.S. Pat. Nos. 5,437,990; 5,409,818; and 5,399,491; the transcription amplification system (TAS) (Kwoh et al., Proc. Natl. Acad. Sci. USA 86: 1173-1177, 1989); and self-sustained sequence replication (3SR) (Guatelli et al., Proc. Natl. Acad. Sci. USA 87: 1874-1878, 1990; WO 92/08800). Alternatively, methods that amplify the probe to detectable levels can be used, such as Q-replicase amplification (Kramer & Lizardi, Nature 339:401-402, 1989; Lomeli et al., Clin. Chem. 35: 1826-1831, 1989). A review of known amplification methods is provided, for example, by Abramson and Myers in Current Opinion in Biotechnology 4:41-47 (1993).

mRNA can be isolated from the sample. The sample can be a tissue sample. The tissue sample can be a tumour biopsy, such as a lymph node biopsy. General methods for mRNA extraction are well known in the art and are disclosed in standard textbooks of molecular biology, including Ausubel et al., Current Protocols of Molecular Biology, John Wiley and Sons (1997). In particular, RNA isolation can be performed using purification kit, buffer set and protease from commercial manufacturers, such as Qiagen, according to the manufacturer's instructions. For example, total RNA from cells in culture can be isolated using Qiagen RNeasy mini-columns. Other commercially available RNA isolation kits include MASTERPURE® Complete DNA and RNA Purification Kit (EPICENTRE®, Madison, Wis.), and Paraffin Block RNA Isolation Kit (Ambion, Inc.). Total RNA from tissue samples can be isolated using RNA Stat-60 (Tel-Test). RNA prepared from tumor can be isolated, for example, by cesium chloride density gradient centrifugation.

In some embodiments, the first step in gene expression profiling by PCR is the reverse transcription of the RNA template into cDNA, followed by its exponential amplification in a PCR reaction. In other embodiments, a combined reverse-transcription-polymerase chain reaction (RT-PCR) reaction may be used, e.g., as described in U.S. Pat. Nos. 5,310,652; 5,322,770; 5,561,058; 5,641,864; and 5,693,517. The two commonly used reverse transcriptases are avilo myeloblastosis virus reverse transcriptase (AMV-RT) and Moloney murine leukemia virus reverse transcriptase (MMLV-RT). The reverse transcription step is typically primed using specific primers, random hexamers, or oligo-dT primers, depending on the circumstances and the goal of expression profiling. For example, extracted RNA can be reverse-transcribed using a GENEAMP™ RNA PCR kit (Perkin Elmer, Calif, USA), following the manufacturer's instructions. The derived cDNA can then be used as a template in the subsequent PCR reaction.

In some embodiments, Real-Time Reverse Transcription-PCR (qRT-PCR) can be used for both the detection and quantification of RNA targets (Bustin, et al., 2005, Clin. Sci., 109:365-379). Examples of qRT-PCR-based methods can be found, for example, in U.S. Pat. No. 7,101,663, which is incorporated by reference herein in its entirety. Instruments for real-time PCR, such as the Applied Biosystems 7500, are available commercially, as are the reagents, such as TaqMan Sequence Detection chemistry.

For example, TagMan® Gene Expression Assays can be used, following the manufacturer's instructions. These kits are pre-formulated gene expression assays for rapid, reliable detection and quantification of human, mouse and rat mRNA transcripts. TaqMan® or 5′-nuclease assay, as described in U.S. Pat. Nos. 5,210,015; 5,487,972; and 5,804,375; and Holland et al., 1988, Proc. Natl. Acad. Sci. USA 88:7276-7280, can be used. TAQMAN® PCR typically utilizes the 5′-nuclease activity of Taq or Tth polymerase to hydrolyze a hybridization probe bound to its target amplicon, but any enzyme with equivalent 5′ nuclease activity can be used. Two oligonucleotide primers are used to generate an amplicon typical of a PCR reaction. A third oligonucleotide, or probe, is designed to detect nucleotide sequence located between the two PCR primers. The probe is non-extendable by Taq DNA polymerase enzyme, and is labeled with a reporter fluorescent dye and a quencher fluorescent dye. Any laser-induced emission from the reporter dye is quenched by the quenching dye when the two dyes are located close together as they are on the probe. During the amplification reaction, the Taq DNA polymerase enzyme cleaves the probe in a template-dependent manner. The resultant probe fragments disassociate in solution, and signal from the released reporter dye is free from the quenching effect of the second fluorophore. One molecule of reporter dye is liberated for each new molecule synthesized, and detection of the unquenched reporter dye provides the basis for quantitative interpretation of the data.

Any method suitable for detecting degradation product can be used in a 5′ nuclease assay. Often, the detection probe is labeled with two fluorescent dyes, one of which is capable of quenching the fluorescence of the other dye. The dyes are attached to the probe, preferably one attached to the 5′ terminus and the other is attached to an internal site, such that quenching occurs when the probe is in an unhybridized state and such that cleavage of the probe by the 5′ to 3′ exonuclease activity of the DNA polymerase occurs in between the two dyes.

Amplification results in cleavage of the probe between the dyes with a concomitant elimination of quenching and an increase in the fluorescence observable from the initially quenched dye. The accumulation of degradation product is monitored by measuring the increase in reaction fluorescence. U.S. Pat. Nos. 5,491,063 and 5,571,673, both incorporated herein by reference, describe alternative methods for detecting the degradation of probe which occurs concomitant with amplification. 5′-Nuclease assay data may be initially expressed as Ct, or the threshold cycle. As discussed above, fluorescence values are recorded during every cycle and represent the amount of product amplified to that point in the amplification reaction. The point when the fluorescent signal is first recorded as statistically significant is the threshold cycle (Ct).

To minimize errors and the effect of sample-to-sample variation, PCR is usually performed using an internal standard. The ideal internal standard is expressed at a constant level among different tissues, and is unaffected by the experimental treatment. RNAs most frequently used to normalize patterns of gene expression are mRNAs for the housekeeping genes glyceraldehyde-3-phosphate-dehydrogenase (GAPDH) and P-actin.

PCR primers and probes are designed based upon intron sequences present in the gene to be amplified. In this embodiment, the first step in the primer/probe design is the delineation of intron sequences within the genes. This can be done by publicly available software, such as the DNA BLAST software developed by Kent, W., Genome Res. 12(4):656-64 (2002), or by the BLAST software including its variations. Subsequent steps follow well established methods of PCR primer and probe design.

In order to avoid non-specific signals, it can be important to mask repetitive sequences within the introns when designing the primers and probes. This can be easily accomplished by using the Repeat Masker program available on-line through the Baylor College of Medicine, which screens DNA sequences against a library of repetitive elements and returns a query sequence in which the repetitive elements are masked. The masked intron sequences can then be used to design primer and probe sequences using any commercially or otherwise publicly available primer/probe design packages, such as Primer Express (Applied Biosystems); MGB assay-by-design (Applied Biosystems); Primer3 (Rozen and Skaletsky (2000) Primer3 on the WWW for general users and for biologist programmers. In: Krawetz S, Misener S (eds) Bioinformatics Methods and Protocols: Methods in Molecular Biology. Humana Press, Totowa, N.J., pp 365-386).

RNA-Seq, also called Whole Transcriptome Shotgun Sequencing (WTSS) refers to the use of high-throughput sequencing technologies to sequence cDNA in order to get information about a sample's RNA content. Publications describing RNA-Seq include: Wang et al., Nature Reviews Genetics 10 (1): 57-63 (January 2009); Ryan et al. BioTechniques 45 (1): 81-94 (2008); and Maher et al., Nature 458 (7234): 97-101 (January 2009); which are hereby incorporated in their entirety.

Differential gene expression can also be identified, or confirmed using the microarray technique. In this method, polynucleotide sequences of interest (including cDNAs and oligonucleotides) are plated, or arrayed, on a microchip substrate. The arrayed sequences are then hybridized with specific DNA probes from cells or tissues of interest.

In an embodiment of the microarray technique, PCR amplified inserts of cDNA clones are applied to a substrate in a dense array. Preferably at least 10,000 nucleotide sequences are applied to the substrate. The microarrayed genes, immobilized on the microchip at 10,000 elements each, are suitable for hybridization under stringent conditions. Fluorescently labeled cDNA probes may be generated through incorporation of fluorescent nucleotides by reverse transcription of RNA extracted from tissues of interest. Labeled cDNA probes applied to the chip hybridize with specificity to each spot of DNA on the array. After stringent washing to remove non-specifically bound probes, the chip is scanned by confocal laser microscopy or by another detection method, such as a CCD camera. Quantitation of hybridization of each arrayed element allows for assessment of corresponding mRNA abundance. With dual color fluorescence, separately labeled cDNA probes generated from two sources of RNA are hybridized pairwise to the array. The relative abundance of the transcripts from the two sources corresponding to each specified gene is thus determined simultaneously. The miniaturized scale of the hybridization affords a convenient and rapid evaluation of the expression pattern for large numbers of genes. Such methods have been shown to have the sensitivity required to detect rare transcripts, which are expressed at a few copies per cell, and to reproducibly detect at least approximately two-fold differences in the expression levels (Schena et al., Proc. Natl. Acad. Sci. USA 93(2): 106-149 (1996)). Microarray analysis can be performed by commercially available equipment, following manufacturer's protocols, such as by using the Affymetrix GENCHIP™ technology, or Incyte's microarray technology.

Serial analysis of gene expression (SAGE) is a method that allows the simultaneous and quantitative analysis of a large number of gene transcripts, without the need of providing an individual hybridization probe for each transcript. First, a short sequence tag (about 10-14 bp) is generated that contains sufficient information to uniquely identify a transcript, provided that the tag is obtained from a unique position within each transcript. Then, many transcripts are linked together to form long serial molecules, that can be sequenced, revealing the identity of the multiple tags simultaneously. The expression pattern of any population of transcripts can be quantitatively evaluated by determining the abundance of individual tags, and identifying the gene corresponding to each tag. For more details see, e.g. Velculescu et al., Science 270:484-487 (1995); and Velculescu et al., Cell 88:243-51 (1997).

The MassARRAY (Sequenom, San Diego, Calif.) technology is an automated, high-throughput method of gene expression analysis using mass spectrometry (MS) for detection. According to this method, following the isolation of RNA, reverse transcription and PCR amplification, the cDNAs are subjected to primer extension. The cDNA-derived primer extension products are purified, and dispensed on a chip array that is pre-loaded with the components needed for MALTI-TOF MS sample preparation. The various cDNAs present in the reaction are quantitated by analyzing the peak areas in the mass spectrum obtained.

mRNA level can also be measured by an assay based on hybridization. A typical mRNA assay method can contain the steps of 1) obtaining surface-bound subject probes; 2) hybridization of a population of mRNAs to the surface-bound probes under conditions sufficient to provide for specific binding (3) post-hybridization washes to remove nucleic acids not bound in the hybridization; and (4) detection of the hybridized mRNAs. The reagents used in each of these steps and their conditions for use may vary depending on the particular application.

Any suitable assay platform can be used to determine the mRNA level in a sample. For example, an assay can be in the form of a dipstick, a membrane, a chip, a disk, a test strip, a filter, a microsphere, a slide, a multiwell plate, or an optical fiber. An assay system can have a solid support on which a nucleic acid corresponding to the mRNA is attached. The solid support can have, for example, a plastic, silicon, a metal, a resin, glass, a membrane, a particle, a precipitate, a gel, a polymer, a sheet, a sphere, a polysaccharide, a capillary, a film a plate, or a slide. The assay components can be prepared and packaged together as a kit for detecting an mRNA.

The nucleic acid can be labeled, if desired, to make a population of labeled mRNAs. In general, a sample can be labeled using methods that are well known in the art (e.g., using DNA ligase, terminal transferase, or by labeling the RNA backbone, etc.; see, e.g., Ausubel, et al., Short Protocols in Molecular Biology, 3rd ed., Wiley & Sons 1995 and Sambrook et al., Molecular Cloning: A Laboratory Manual, Third Edition, 2001 Cold Spring Harbor, N.Y.). In some embodiments, the sample is labeled with fluorescent label. Exemplary fluorescent dyes include but are not limited to xanthene dyes, fluorescein dyes, rhodamine dyes, fluorescein isothiocyanate (FITC), 6 carboxyfluorescein (FAM), 6 carboxy-2′,4′,7′,4,7-hexachlorofluorescein (HEX), 6 carboxy 4′,5′ dichloro 2′,7′ dimethoxyfluorescein (JOE or J), N,N,N′,N′ tetramethyl 6 carboxyrhodamine (TAMRA or T), 6 carboxy X rhodamine (ROX or R), 5 carboxyrhodamine 6G (R6G5 or G5), 6 carboxyrhodamine 6G (R6G6 or G6), and rhodamine 110; cyanine dyes, e.g. Cy3, Cy5 and Cy7 dyes; Alexa dyes, e.g. Alexa-fluor-555; coumarin, Diethylaminocoumarin, umbelliferone; benzimide dyes, e.g. Hoechst 33258; phenanthridine dyes, e.g. Texas Red; ethidium dyes; acridine dyes; carbazole dyes; phenoxazine dyes; porphyrin dyes; polymethine dyes, BODIPY dyes, quinoline dyes, Pyrene, Fluorescein Chlorotriazinyl, R110, Eosin, JOE, R6G, Tetramethylrhodamine, Lissamine, ROX, Napthofluorescein, and the like.

Hybridization can be carried out under suitable hybridization conditions, which may vary in stringency as desired. Typical conditions are sufficient to produce probe/target complexes on a solid surface between complementary binding members, i.e., between surface-bound subject probes and complementary mRNAs in a sample. In certain embodiments, stringent hybridization conditions can be employed.

Hybridization is typically performed under stringent hybridization conditions. Standard hybridization techniques (e.g. under conditions sufficient to provide for specific binding of target mRNAs in the sample to the probes) are described in Kallioniemi et al., Science 258:818-821 (1992) and WO 93/18186. Several guides to general techniques are available, e.g., Tijssen, Hybridization with Nucleic Acid Probes, Parts I and II (Elsevier, Amsterdam 1993). For descriptions of techniques suitable for in situ hybridizations, see Gall et al. Meth. Enzymol., 21:470-480 (1981); and Angerer et al. in Genetic Engineering: Principles and Methods (Setlow and Hollaender, Eds.) Vol 7, pgs 43-65 (Plenum Press, New York 1985). Selection of appropriate conditions, including temperature, salt concentration, polynucleotide concentration, hybridization time, stringency of washing conditions, and the like will depend on experimental design, including source of sample, identity of capture agents, degree of complementarity expected, etc., and may be determined as a matter of routine experimentation for those of ordinary skill in the art. Those of ordinary skill will readily recognize that alternative but comparable hybridization and wash conditions can be utilized to provide conditions of similar stringency.

After the mRNA hybridization procedure, the surface bound polynucleotides are typically washed to remove unbound nucleic acids. Washing may be performed using any convenient washing protocol, where the washing conditions are typically stringent, as described above. The hybridization of the target mRNAs to the probes is then detected using standard techniques.

Any methods as described herein or otherwise known in the art can be used to determine the mRNA level of a gene in a sample from a subject described herein. By way of example, in some embodiments, provided herein are methods to treat AML in a subject that include determining the mRNA levels of CXCL12 and IGF1 in a sample from the subject by using qRT-PCR, and administering a therapeutically effective amount of tipifarnib to the subject if the mRNA level of CXCL12 in the sample is higher than a reference mRNA level of the CXCL12 and if the mRNA level of IGF1 in the sample is lower than a reference mRNA level of IGF1.

In some embodiments, provided herein are methods of treating a cancer in a subject by administering a therapeutically effective amount of an FTI based on the protein level or mutation status of certain genes as described herein. In some embodiments, provided herein are methods of treating a cancer in a subject by administering a therapeutically effective amount of FTI (e.g. tipifarnib) to the subject, wherein the subject has a greater CXCL12 protein level than a reference level of CXCL12 protein, and a lower IGF1 protein level than a reference protein level of IGF1. In some embodiments, the subject has an IGF1 protein level that is non-detectable. In some embodiments, provided herein are methods of treating a cancer in a subject by administering a therapeutically effective amount of FTI (e.g. tipifarnib) to the subject, wherein the subject has a greater CXCL12 protein level than a reference protein level of CXCL12, and a greater IGFBP7 protein level than a reference protein level of IGFBP7.

In some embodiments, the subject selected for an FTI (e.g. tipifarnib) treatment further does not carry the IGFBP7 variant L11F (rs11573021). In some embodiments, provided herein are methods of treating a cancer in a subject by administering a therapeutically effective amount of FTI (e.g. tipifarnib) to the subject, wherein the subject has a greater CXCL12 protein level than a reference protein level of CXCL12 expression, and wherein the subject further does not carry the IGFBP7 variant L11F (rs11573021). In some embodiments, provided herein are methods of treating a cancer in a subject by administering a therapeutically effective amount of FTI (e.g. tipifarnib) to the subject, wherein the subject has a greater CXCL12 protein level than a reference protein level of CXCL12, and wherein the subject further does not carry an activating mutation in PIK3CA. In some embodiments, provided herein are methods of treating a cancer in a subject by administering a therapeutically effective amount of FTI (e.g. tipifarnib) to the subject, wherein the subject has a greater CXCL12 protein level than a reference protein level of CXCL12, and wherein the subject further does not carry an activating mutation in AKT.

In some embodiments, provided herein are methods of treating a cancer in a subject by administering a therapeutically effective amount of FTI (e.g. tipifarnib) to the subject, wherein the subject has a greater CXCL12 protein level than a reference protein level of CXCL12, and wherein the subject has (1) a lower IGF1 protein level than a reference protein level of IGF1, or (2) a greater IGFBP7 protein level than a reference protein level of IGFBP7, wherein the subject further does not carry the IGFBP7 variant L11F (rs11573021), an activating mutation in PIK3CA, and/or activating mutation in AKT.

Accordingly, the methods provided herein for selecting subjects having a cancer for an FTI (e.g. tipifarnib) treatment further include measuring IGF2 protein level in the subject. In some embodiments, provided herein are methods of treating a cancer in a subject by administering a therapeutically effective amount of FTI (e.g. tipifarnib) to the subject, wherein the subject has a greater CXCL12 protein level than a reference protein level of CXCL12, and a lower IGF2 protein level than a reference protein level of IGF2. In some embodiments, the subject has an IGF2 protein level that is non-detectable. In some embodiments, provided herein are methods of treating cancer in a subject by administering a therapeutically effective amount of FTI (e.g. tipifarnib) to the subject, wherein the subject has a greater CXCL12 protein level than a reference protein level of CXCL12, and a greater IGF2R protein level than a reference protein level of IGF2R. In some embodiments, the subject does not have a loss of heterozygosity of IGF2. In some embodiments, the subject does not have a loss of imprinting of IGF2. In some embodiments, the subject does not have a loss of heterozygosity of IGF2 or a loss of imprinting of IGF2.

In some embodiments, methods provided herein use both IGF1 and IGF2 protein levels to select cancer patients for an FTI (e.g. tipifarnib) treatment. In some embodiments, provided herein are methods of treating a cancer in a subject by administering a therapeutically effective amount of FTI (e.g. tipifarnib) to the subject, wherein the subject has a greater CXCL12 protein level than a reference protein level of CXCL12, a lower IGF1 protein level than a reference protein level of IGF1, and a lower IGF2 protein level than a reference protein level of IGF2. In some embodiments, the subject has an IGF1 protein level that is non-detectable. In some embodiments, the subject has an IGF2 protein level that is non-detectable. In some embodiments, the subject has an IGF1 protein level that is non-detectable and an IGF2 protein level that is non-detectable.

The protein ratio of CXCL12 to CXCR4 also indicates the activation of the CXCL12/CXCR4 pathway, and can predict the likelihood of responsiveness of a cancer to an FTI (e.g. tipifarnib) treatment. As such, provided herein are also methods to select cancer patients for an FTI treatment, methods to predict the responsiveness of an FTI treatment in a cancer patient, and methods to increase the responsiveness to an FTI treatment in a cancer patient population based the protein ratio of CXCL12 to CXCR4. In some embodiments, provided herein are methods of treating cancer in a subject by administering a therapeutically effective amount of an FTI to the subject, wherein the subject has a greater ratio of CXCL12 protein level to CXCR4 protein level than a reference ratio, and an inactive IGF1R pathway.

The subject having an inactive IGF1R pathway can have a lower IGF1 protein level than a reference protein level of IGF1, or a greater IGFBP7 protein level than a reference protein level of IGFBP7. In some embodiments, provided herein are methods of treating a cancer in a subject by administering a therapeutically effective amount of an FTI (e.g. tipifarnib) to the subject, wherein the subject has a greater ratio of CXCL12 protein level to CXCR4 protein level than a reference ratio, and a lower IGF1 protein level than a reference protein level of IGF1. In some embodiments, the subject has an IGF1 protein level that is non-detectable. In some embodiments, provided herein are methods of treating cancer in a subject by administering a therapeutically effective amount of an FTI (e.g. tipifarnib) to the subject, wherein the subject has a greater ratio of CXCL12 protein level to CXCR4 protein level than a reference ratio, and a greater IGFBP7 protein level than a reference protein level of IGFBP7.

In some embodiments, provided herein are methods of treating cancer in a subject by administering a therapeutically effective amount of FTI (e.g. tipifarnib) to the subject, wherein the subject has a greater ratio of CXCL12 protein level to CXCR4 protein level than a reference ratio, and wherein the subject further does not carry the IGFBP7 variant L11F (rs11573021). In some embodiments, provided herein are methods of treating cancer in a subject by administering a therapeutically effective amount of FTI (e.g. tipifarnib) to the subject, wherein the subject has a greater ratio of CXCL12 protein level to CXCR4 protein level than a reference ratio, and wherein the subject further does not carry an activating mutation in PIK3CA. In some embodiments, provided herein are methods of treating cancer in a subject by administering a therapeutically effective amount of FTI (e.g. tipifarnib) to the subject, wherein the subject has a greater ratio of CXCL12 protein level to CXCR4 protein level than a reference ratio, and wherein the subject further does not carry an activating mutation in AKT.

In some embodiments, provided herein are methods of treating cancer in a subject by administering a therapeutically effective amount of FTI (e.g. tipifarnib) to the subject, wherein the subject has a greater ratio of CXCL12 protein level to CXCR4 protein level than a reference ratio, and wherein the subject has (1) a lower IGF1 protein level than a reference protein level of IGF1, or (2) a greater IGFBP7 protein level than a reference protein level of IGFBP7, wherein the subject further does not carry the IGFBP7 variant L11F (rs11573021), an activating mutation in PIK3CA, and/or activating mutation in AKT.

In some embodiments, the subjects selected for an FTI (e.g. tipifarnib) treatment based on a greater ratio of CXCL12 protein level to CXCR4 protein level than a reference ratio further has a lower IGF2 protein level than a reference protein level of IGF2. In some embodiments, the subject has an IGF2 protein level that is non-detectable. In some embodiments, provided herein are methods of treating cancer in a subject by administering a therapeutically effective amount of an FTI (e.g. tipifarnib) to the subject, wherein the subject has a greater ratio of CXCL12 protein level to CXCR4 protein level, and a greater IGF2R protein level than a reference protein level of IGF2R. In some embodiments, the subject does not have a loss of heterozygosity of IGF2. In some embodiments, the subject does not have a loss of imprinting of IGF2. In some embodiments, the subject does not have a loss of heterozygosity of IGF2 or a loss of imprinting of IGF2.

In some embodiments, the subjects selected for an FTI (e.g. tipifarnib) treatment based on a greater ratio of CXCL12 protein level to CXCR4 protein level than a reference ratio further has a lower IGF1 protein level than a reference protein level of IGF1, and a lower IGF2 protein level than a reference protein level of IGF2. In some embodiments, the subject has an IGF1 protein level that is non-detectable. In some embodiments, the subject has an IGF2 protein level that is non-detectable. In some embodiments, the subject has an IGF1 protein level that is non-detectable and an IGF2 protein level that is non-detectable.

In addition, the protein level of CXCR4 can also predict the likelihood of responsiveness of a cancer to an FTI (e.g. tipifarnib) treatment. As such, provided herein are also methods to select cancer patients for an FTI (e.g. tipifarnib) treatment, methods to predict the responsiveness of an FTI (e.g. tipifarnib) treatment in a cancer patient, and methods to increase the responsiveness to an FTI (e.g. tipifarnib) treatment in a cancer patient population based the protein level of CXCR4. In some embodiments, provided herein are methods of treating cancer in a subject by administering a therapeutically effective amount of an FTI (e.g. tipifarnib) to the subject, wherein the subject has a greater protein level of CXCR4 than a reference protein level of CXCR4, and an inactive IGF1R pathway. In some embodiments, provided herein are methods of treating cancer in a subject by administering a therapeutically effective amount of an FTI (e.g. tipifarnib) to the subject, wherein the subject has a greater ratio of CXCR4 protein level to CXCR2 protein level than a reference ratio, and an inactive IGF1R pathway. In some embodiments, provided herein are methods of treating cancer in a subject by administering a therapeutically effective amount of an FTI (e.g. tipifarnib) to the subject, wherein the subject has an activating mutation in CXCR4, and an inactive IGF1R pathway.

The subject having an inactive IGF1R pathway can have a lower IGF1 protein level than a reference protein level of IGF1. In some embodiments, provided herein are methods of treating cancer in a subject by administering a therapeutically effective amount of an FTI (e.g. tipifarnib) to the subject, wherein the subject has a greater protein level of CXCR4 than a reference protein level of CXCR4, and a lower IGF1 protein level than a reference protein level of IGF1. In some embodiments, provided herein are methods of treating cancer in a subject by administering a therapeutically effective amount of an FTI (e.g. tipifarnib) to the subject, wherein the subject has a greater ratio of CXCR4 protein level to CXCR2 protein level than a reference ratio, and a lower IGF1 protein level than a reference protein level of IGF1. In some embodiments, provided herein are methods of treating cancer in a subject by administering a therapeutically effective amount of an FTI (e.g. tipifarnib) to the subject, wherein the subject has an activating mutation of CXCR4, and a lower IGF1 protein level than a reference protein level of IGF1. In some embodiments, the subject has an IGF1 protein level that is non-detectable.

The subject having an inactive IGF1R pathway can be a greater IGFBP7 protein level than a protein level of IGFBP7. In some embodiments, provided herein are methods of treating a cancer in a subject by administering a therapeutically effective amount of an FTI (e.g. tipifarnib) to the subject, wherein the subject has a greater protein level of CXCR4 than a reference protein level of CXCR4, and a greater IGFBP7 protein level than a reference protein level of IGFBP7. In some embodiments, provided herein are methods of treating cancer in a subject by administering a therapeutically effective amount of an FTI (e.g. tipifarnib) to the subject, wherein the subject has a greater ratio of CXCR4 protein level to CXCR2 protein level than a reference ratio, and a greater IGFBP7 protein level than a reference protein level of IGFBP7. In some embodiments, provided herein are methods of treating cancer in a subject by administering a therapeutically effective amount of an FTI (e.g. tipifarnib) to the subject, wherein the subject has an activating mutation of CXCR4, and a greater IGFBP7 protein level than a reference protein level of IGFBP7.

In some embodiments, the subjects selected for an FTI (e.g. tipifarnib) treatment based on a greater ratio of CXCL12 protein level to CXCR4 protein level than a reference ratio further has a lower IGF2 protein level than a reference protein level of IGF2. In some embodiments, the subject has an IGF2 protein level that is non-detectable. In some embodiments, provided herein are methods of treating cancer in a subject by administering a therapeutically effective amount of an FTI (e.g. tipifarnib) to the subject, wherein the subject has a greater ratio of CXCL12 protein level to CXCR4 protein level, and a greater IGF2R protein level than a reference protein level of IGF2R. In some embodiments, the subject does not have a loss of heterozygosity of IGF2. In some embodiments, the subject does not have a loss of imprinting of IGF2. In some embodiments, the subject does not have a loss of heterozygosity of IGF2 or a loss of imprinting of IGF2.

In some embodiments, the subjects selected for an FTI (e.g. tipifarnib) treatment based on a greater ratio of CXCL12 protein level to CXCR4 protein level than a reference ratio further has a lower IGF1 protein level than a reference protein level of IGF1, and a lower IGF2 protein level than a reference protein level of IGF2. In some embodiments, the subject has an IGF1 protein level that is non-detectable. In some embodiments, the subject has an IGF2 protein level that is non-detectable. In some embodiments, the subject has an IGF1 protein level that is non-detectable and an IGF2 protein level that is non-detectable.

In some embodiments, provided herein are methods for selecting cancer patients for an FTI (e.g. tipifarnib) treatment wherein the patients have (1) either CXCL12 activation (high CXCL12 protein level or high ratio of CXCL12 protein level/CXCR4 protein level) or CXCR4 activation (high CXCR4 protein level, high ratio of CXCR4 protein level/CXCR2 protein level, or activating mutation in CXCR4), and (2) inactive IGF1R pathway (low IGF1 protein level, low IGF2 protein level, high IGFBP7 protein level, and/or absence of activating mutation in PIK3CA and AKT) and/or low mutation allele frequency for TP53 and/or KRAS.

The FTI can be any FTI, including those described herein. For example, the FTI can be tipifarnib, lonafarnib, arglabin, perrilyl alcohol, L778123, L739749, FTI-277, L744832, CP-609,754, R208176, AZD3409, or BMS-214662. In some embodiments, the FTI is tipifarnib. As such, provided herein are methods to select cancer patients for tipifarnib treatment, methods to predict the responsiveness to tipifarnib treatment in a cancer patient, and methods to increase the overall responsiveness of tipifarnib treatment in a cancer patient population, based on the protein levels of molecular biomarkers disclosed herein, including, for example, the protein levels of CXCL12, IGF1, IGFBP7, IGF2, IGF2R, CXCR4, CXCR2, or any combination thereof.

In some embodiments, the reference protein level of a gene is the median protein level of the gene in a population of healthy subjects. In some embodiments, the reference protein level of a gene is the median protein level of the gene in a population of subjects having a particular cancer. For example, the reference protein level of a gene in pancreatic cancer patients is the median protein level of the gene in a population of subjects having pancreatic cancer. For another example, the reference protein level of a gene in AML patients is the median protein level of the gene in a population of subjects having AML. In some embodiments, the reference protein level of a gene is the cutoff value determined by statistical analysis by a person of ordinary skill in the art.

Methods to determine a protein level of a gene in a sample are well known in the art. For example, in some embodiments, the protein level can be determined by an immunohistochemistry (IHC) assay, an immunoblotting (IB) assay, an immunofluorescence (IF) assay, flow cytometry (FACS), or an Enzyme-Linked Immunosorbent Assay (ELISA). In some embodiments, the protein level can be determined by Hematoxylin and Eosin stain (“H&E staining”).

The protein level of the gene can be detected by a variety of (IHC) approaches or other immunoassay methods. IHC staining of tissue sections has been shown to be a reliable method of assessing or detecting presence of proteins in a sample. Immunohistochemistry techniques utilize an antibody to probe and visualize cellular antigens in situ, generally by chromogenic or fluorescent methods. Thus, antibodies or antisera, including for example, polyclonal antisera, or monoclonal antibodies specific for each gene are used to detect expression. As discussed in greater detail below, the antibodies can be detected by direct labeling of the antibodies themselves, for example, with radioactive labels, fluorescent labels, hapten labels such as, biotin, or an enzyme such as horse radish peroxidase or alkaline phosphatase. Alternatively, unlabeled primary antibody is used in conjunction with a labeled secondary antibody, comprising antisera, polyclonal antisera or a monoclonal antibody specific for the primary antibody. Immunohistochemistry protocols and kits are well known in the art and are commercially available. Automated systems for slide preparation and IHC processing are available commercially. The Ventana® BenchMark XT system is an example of such an automated system.

Standard immunological and immunoassay procedures can be found in Basic and Clinical Immunology (Stites & Terr eds., 7th ed. 1991). Moreover, the immunoassays can be performed in any of several configurations, which are reviewed extensively in Enzyme Immunoassay (Maggio, ed., 1980); and Harlow & Lane, supra. For a review of the general immunoassays, see also Methods in Cell Biology: Antibodies in Cell Biology, volume 37 (Asai, ed. 1993); Basic and Clinical Immunology (Stites & Ten, eds., 7th ed. 1991).

Commonly used assays to detect protein level of a gene include noncompetitive assays, e.g., sandwich assays, and competitive assays. Typically, an assay such as an ELISA assay can be used. ELISA assays are known in the art, e.g., for assaying a wide variety of tissues and samples, including blood, plasma, serum, a tumor biopsy, a lymph node, or bone marrow. In some embodiments, the sample is a bone marrow biopsy. In some embodiments, the sample is a bone marrow aspirate. In some embodiments, the sample can be a spinal fluid sample, a liver sample, a testicle sample, a spleen sample, or a lymph node sample. In some embodiments, the sample is isolated cells.

A wide range of immunoassay techniques using such an assay format are available, see, e.g., U.S. Pat. Nos. 4,016,043, 4,424,279, and 4,018,653, which are hereby incorporated by reference in their entireties. These include both single-site and two-site or “sandwich” assays of the non-competitive types, as well as in the traditional competitive binding assays. These assays also include direct binding of a labeled antibody to a target gene. Sandwich assays are commonly used assays. A number of variations of the sandwich assay technique exist. For example, in a typical forward assay, an unlabeled antibody is immobilized on a solid substrate, and the sample to be tested brought into contact with the bound molecule. After a suitable period of incubation, for a period of time sufficient to allow formation of an antibody-antigen complex, a second antibody specific to the antigen, labeled with a reporter molecule capable of producing a detectable signal is then added and incubated, allowing time sufficient for the formation of another complex of antibody-antigen-labeled antibody. Any unreacted material is washed away, and the presence of the antigen is determined by observation of a signal produced by the reporter molecule. The results may either be qualitative, by simple observation of the visible signal, or may be quantitated by comparing with a control sample containing known amounts of the gene.

Variations on the forward assay include a simultaneous assay, in which both sample and labeled antibody are added simultaneously to the bound antibody. These techniques are well known to those skilled in the art, including any minor variations as will be readily apparent. In a typical forward sandwich assay, a first antibody having specificity for the gene is either covalently or passively bound to a solid surface. The solid surface may be glass or a polymer, the most commonly used polymers being cellulose, polyacrylamide, nylon, polystyrene, polyvinyl chloride, or polypropylene. The solid supports may be in the form of tubes, beads, discs of microplates, or any other surface suitable for conducting an immunoassay. The binding processes are well-known in the art and generally consist of cross-linking covalently binding or physically adsorbing, the polymer-antibody complex is washed in preparation for the test sample. An aliquot of the sample to be tested is then added to the solid phase complex and incubated for a period of time sufficient (e.g. 2-40 minutes or overnight if more convenient) and under suitable conditions (e.g., from room temperature to 40° C. such as between 25° C. and 32° C. inclusive) to allow binding of any subunit present in the antibody. Following the incubation period, the antibody subunit solid phase is washed and dried and incubated with a second antibody specific for a portion of the gene. The second antibody is linked to a reporter molecule which is used to indicate the binding of the second antibody to the molecular marker.

An alternative method involves immobilizing the target gene in the sample and then exposing the immobilized target to specific antibody which may or may not be labeled with a reporter molecule. Depending on the amount of target and the strength of the reporter molecule signal, a bound target may be detectable by direct labeling with the antibody. Alternatively, a second labeled antibody, specific to the first antibody is exposed to the target-first antibody complex to form a target-first antibody-second antibody tertiary complex. The complex is detected by the signal emitted by a labeled reporter molecule.

In the case of an enzyme immunoassay, an enzyme is conjugated to the second antibody, generally by means of glutaraldehyde or periodate. As will be readily recognized, however, a wide variety of different conjugation techniques exist, which are readily available to the skilled artisan. Commonly used enzymes include horseradish peroxidase, glucose oxidase, beta-galactosidase, and alkaline phosphatase, and other are discussed herein. The substrates to be used with the specific enzymes are generally chosen for the production, upon hydrolysis by the corresponding enzyme, of a detectable color change. Examples of suitable enzymes include alkaline phosphatase and peroxidase. It is also possible to employ fluorogenic substrates, which yield a fluorescent product rather than the chromogenic substrates noted above. In all cases, the enzyme-labeled antibody is added to the first antibody-molecular marker complex, allowed to bind, and then the excess reagent is washed away. A solution containing the appropriate substrate is then added to the complex of antibody-antigen-antibody. The substrate will react with the enzyme linked to the second antibody, giving a qualitative visual signal, which may be further quantitated, usually spectrophotometrically, to give an indication of the amount of gene which was present in the sample. Alternately, fluorescent compounds, such as fluorescein and rhodamine, can be chemically coupled to antibodies without altering their binding capacity. When activated by illumination with light of a particular wavelength, the fluorochrome-labeled antibody adsorbs the light energy, inducing a state to excitability in the molecule, followed by emission of the light at a characteristic color visually detectable with a light microscope. As in the EIA, the fluorescent labeled antibody is allowed to bind to the first antibody-molecular marker complex. After washing off the unbound reagent, the remaining tertiary complex is then exposed to the light of the appropriate wavelength, the fluorescence observed indicates the presence of the molecular marker of interest. Immunofluorescence and EIA techniques are both very well established in the art and are discussed herein.

Any methods as described herein or otherwise known in the art can be used to determine the protein level of a gene in a sample from a subject described herein. By way of example, in some embodiments, provided herein are methods to treat pancreatic cancer in a subject that include determining the protein levels of CXCL12, CXCR4, and IGFBP7 in a sample from the subject by using an IF assay, and administering a therapeutically effective amount of tipifarnib to the subject if the protein ratio of CXCL12 to CXCR4 in the sample is higher than a reference ratio, and if the protein level of IGFBP7 in the sample is greater than a reference protein level of IGFBP7.

Any methods for analyzing expression levels (e.g., the protein level or the mRNA level) as described herein or otherwise known in the art can be used to determine the level of the additional gene in a sample, such as an IHC assay, an D3 assay, an IF assay, FACS, ELISA, protein microarray analysis, qPCR, qRT-PCR, RNA-seq, RNA microarray analysis, SAGE, MassARRAY technique, next-generation sequencing, or FISH.

In some embodiments, the methods described herein can further include determining the mutation status of particular genes, such as IGFBP7, KRAS, TP53, PIK3CA, AKT, CXCR4, or any combinations thereof. In some embodiments, the methods provided herein further include determining the mutation status of IGFBP7. In some embodiments, the methods provided herein further include determining the mutation status of KRAS. In some embodiments, the methods provided herein further include determining the mutation status of TP53. In some embodiments, the methods provided herein further include determining the mutation status of PIK3CA. In some embodiments, the methods provided herein further include determining the mutation status of AKT. In some embodiments, the methods provided herein further include determining the mutation status of CXCR4.

In some embodiments, the methods described herein can further include determining the mutation allele frequencies of particular genes, such as IGFBP7, KRAS, TP53, PIK3CA, AKT, CXCR4, or any combinations thereof. In some embodiments, the methods provided herein further include determining the mutation allele frequency of IGFBP7. In some embodiments, the methods provided herein further include determining the mutation allele frequency of KRAS. In some embodiments, the methods provided herein further include determining the mutation allele frequency of TP53. In some embodiments, the methods provided herein further include determining the mutation allele frequency of PIK3CA. In some embodiments, the methods provided herein further include determining the mutation allele frequency of AKT. In some embodiments, the methods provided herein further include determining the mutation allele frequency of CXCR4.

Methods for determining mutation status and/or mutation allele frequency are well known in the art. In some embodiments, the methods include sequencing, Polymerase Chain Reaction (PCR), DNA microarray, Mass Spectrometry (MS), Single Nucleotide Polymorphism (SNP) assay, denaturing high-performance liquid chromatography (DHPLC), or Restriction Fragment Length Polymorphism (RFLP) assay. In some embodiments, the mutation status and/or mutation allele frequency can be determined using standard sequencing methods, including, for example, Sanger sequencing, next generation sequencing (NGS). In some embodiments, the mutation status and/or mutation allele frequency can be determined an NGS-based assay. In some embodiments, the mutation status and/or mutation allele frequency can be determined by a qualitative PCR-based assay. In some embodiments, the mutation status and/or mutation allele frequency can be determined by a real-time PCR assay. In some embodiments, the mutation status and/or mutation allele frequency is determined using MS. In some embodiments, the mutation status and/or mutation allele frequency is determined by analyzing protein obtained from the sample. For example, the mutation status can be detected by a variety of immunohistochemistry (IHC) approaches, Immunoblotting assay, Enzyme-Linked Immunosorbent Assay (ELISA) or other immunoassay methods known in the art.

As a person of ordinary skill in the art would understand, any methods described herein or otherwise known in the art for analyzing mutation status or of a gene can be used in the methods described herein to determining the presence or absence of specific mutation(s).

In some embodiments, the methods provided herein also include obtaining a sample from the subject. The sample used in the methods provided herein includes body fluids from a subject or a tumour biopsy from the subject.

In some embodiments, the sample used in the present methods includes a biopsy (e.g., a tumor biopsy). The biopsy can be from any organ or tissue, for example, skin, liver, lung, heart, colon, kidney, bone marrow, teeth, lymph node, hair, spleen, brain, breast, or other organs. Any biopsy technique known by those skilled in the art can be used for isolating a sample from a subject, for instance, open biopsy, close biopsy, core biopsy, incisional biopsy, excisional biopsy, or fine needle aspiration biopsy. In some embodiments, the sample used in the present methods includes an aspirate (e.g., bone marrow aspirate). In some embodiments, the sample is a lymph node biopsy. In some embodiments, the sample can be a frozen tissue sample. In some embodiments, the sample can be a formalin-fixed paraffin-embedded (“FFPE”) tissue sample. In some embodiments, the sample can be a deparaffinised tissue section. In some embodiments, the sample can be a liver sample. In some embodiments, the sample can be a testicle sample. In some embodiments, the sample can be a spleen sample. In some embodiments, the sample can be a lymph node sample.

In some embodiments, the sample is a body fluid sample. Non-limiting examples of body fluids include blood (e.g., peripheral whole blood, peripheral blood), blood plasma, bone marrow, amniotic fluid, aqueous humor, bile, lymph, menses, serum, urine, cerebrospinal fluid surrounding the brain and the spinal cord, synovial fluid surrounding bone joints. In some embodiments, the sample can be a spinal fluid sample.

In some embodiments, the sample is a blood sample. The blood sample can be a whole blood sample, a partially purified blood sample, or a peripheral blood sample. The blood sample can be obtained using conventional techniques as described in, e.g. Innis et al, editors, PCR Protocols (Academic Press, 1990). White blood cells can be separated from blood samples using convention techniques or commercially available kits, e.g. RosetteSep kit (Stein Cell Technologies, Vancouver, Canada). Sub-populations of white blood cells, e.g. mononuclear cells, NK cells, B cells, T cells, monocytes, granulocytes or lymphocytes, can be further isolated using conventional techniques, e.g. magnetically activated cell sorting (MACS) (Miltenyi Biotec, Auburn, Calif.) or fluorescently activated cell sorting (FACS) (Becton Dickinson, San Jose, Calif.). In some embodiments, the sample is serum. In some embodiments, the sample is plasma. In one embodiment, the sample is a bone marrow sample.

In certain embodiments, the sample used in the methods provided herein includes a plurality of cells. Such cells can include any type of cells, e.g., stem cells, blood cells (e.g., PBMCs), lymphocytes, NK cells, B cells, T cells, monocytes, granulocytes, immune cells, or tumor or cancer cells. Specific cell populations can be obtained using a combination of commercially available antibodies (e.g., Quest Diagnostic (San Juan Capistrano, Calif.); Dako (Denmark)). In some embodiments, the sample is isolated cells.

In certain embodiments, the sample used in the methods provided herein includes a plurality of cells from the diseased tissue, e.g., a tumor sample. In some embodiments, the cells can be obtained from the tumor tissue, such as a tumor biopsy or a tumor explants. In certain embodiments, the number of cells used in the methods provided herein can range from a single cell to about 10⁹ cells. In some embodiments, the number of cells used in the methods provided herein is about 1×10⁴, 5×10⁴, 1×10⁵, 5×10⁵, 1×10⁶, 5×10⁶, 1×10⁷, 5×10⁷, 1×10⁸, or 5×10⁸. Different types of procedures are available to obtain a tumor biopsy from a patient, including skin biopsy, shave (tangential) biopsy, punch biopsy, incisional biopsy (which removes a portion of the tumor) and excisional biopsy (which removes the entire tumor). Lymph node biopsies are usually performed to examiner whether cancer has spread. Both fine needle aspiration (FNA) biopsy and surgical (excisional) lymph node biopsy are available options. The FNA biopsy allows the patient to use a thin needle to obtain a small fragment of the lymph node, which is less invasive than the surgical option, but may not always provide a large enough sample to find cancer cells.

The number and type of cells collected from a subject can be monitored, for example, by measuring changes in morphology and cell surface markers using standard cell detection techniques such as flow cytometry, cell sorting, immunocytochemistry (e.g., staining with tissue specific or cell-marker specific antibodies) fluorescence activated cell sorting (FACS), magnetic activated cell sorting (MACS), by examination of the morphology of cells using light or confocal microscopy, and/or by measuring changes in gene expression using techniques well known in the art, such as PCR and gene expression profiling. These techniques can be used, too, to identify cells that are positive for one or more particular markers. Fluorescence activated cell sorting (FACS) is a well-known method for separating particles, including cells, based on the fluorescent properties of the particles (Kamarch, 1987, Methods Enzymol, 151:150-165). Laser excitation of fluorescent moieties in the individual particles results in a small electrical charge allowing electromagnetic separation of positive and negative particles from a mixture. In one embodiment, cell surface marker-specific antibodies or ligands are labeled with distinct fluorescent labels. Cells are processed through the cell sorter, allowing separation of cells based on their ability to bind to the antibodies used. FACS sorted particles may be directly deposited into individual wells of 96-well or 384-well plates to facilitate separation and cloning.

In certain embodiments, subsets of cells are used in the methods provided herein. Methods to sort and isolate specific populations of cells are well-known in the art and can be based on cell size, morphology, or intracellular or extracellular markers. Such methods include, but are not limited to, flow cytometry, flow sorting, FACS, bead based separation such as magnetic cell sorting, size-based separation (e.g., a sieve, an array of obstacles, or a filter), sorting in a microfluidics device, antibody-based separation, sedimentation, affinity adsorption, affinity extraction, density gradient centrifugation, laser capture microdissection, etc

### 1.4. Reference Level

As described herein, the term “reference expression level” when used in connection with a gene (e.g. CXCL12, IGF1, IGFBP7, IGF2, IGF2R, CXCR4, or CXCR2) refers to a predetermined expression level of the gene that one can use to determine the significance of the expression level of the gene in a sample. The sample can be a cell, a group of cells, or a tissue. A reference expression level of a gene (e.g. CXCL12, IGF1, IGFBP7, IGF2, IGF2R, CXCR4, or CXCR2) can be the median expression level of the gene in a population of healthy subjects. In some embodiments, the reference expression level of a gene (e.g. CXCL12, IGF1, IGFBP7, IGF2, IGF2R, CXCR4, or CXCR2) can be the median expression level of the gene in a population of subjects having the same type of tumor.

As such, in some embodiments, a reference expression level of CXCL12 can be the median expression level of CXCL12 in a population of healthy subjects. In some embodiments, the reference expression level of CXCL12 can be the median expression level of CXCL12 in a population of subjects having the same type of tumor. In some embodiments, a reference expression level of IGF1 can be the median expression level of IGF1 in a population of healthy subjects. In some embodiments, the reference expression level of IGF1 can be the median expression level of IGF1 in a population of subjects having the same type of tumor. In some embodiments, a reference expression level of IGFBP7 can be the median expression level of IGFBP7 in a population of healthy subjects. In some embodiments, the reference expression level of IGFBP7 can be the median expression level of IGFBP7 in a population of subjects having the same type of tumor. In some embodiments, a reference expression level of IGF2 can be the median expression level of IGF2 in a population of healthy subjects. In some embodiments, the reference expression level of IGF2 can be the median expression level of IGF2 in a population of subjects having the same type of tumor. In some embodiments, a reference expression level of IGF2R can be the median expression level of IGF2R in a population of healthy subjects. In some embodiments, the reference expression level of IGF2R can be the median expression level of IGF2R in a population of subjects having the same type of tumor. In some embodiments, a reference expression level of CXCR4 can be the median expression level of CXCR4 in a population of healthy subjects. In some embodiments, the reference expression level of CXCR4 can be the median expression level of CXCR4 in a population of subjects having the same type of tumor. In some embodiments, a reference expression level of CXCR2 can be the median expression level of CXCR2 in a population of healthy subjects. In some embodiments, the reference expression level of CXCR2 can be the median expression level of CXCR2 in a population of subjects having the same type of tumor.

A reference expression level of a gene (e.g. CXCL12, IGF1, IGFBP7, IGF2, IGF2R, CXCR4, or CXCR2) can be a cut-off value determined by a person of ordinary skill in the art through statistical analysis of the expression levels of the gene in various sample cell populations. In some embodiments, the reference expression level of a gene can be a cutoff percentile of the expression in a population of subjects having the same type of tumor. The cutoff percentile can be the top 10%, 15%, 20%, 25%, 30%, 35%, 40%, 45%, or 50% cutoff. The cutoff percentile can be the top 10% cutoff. The cutoff percentile (e.g. CXCL12, IGF1, IGFBP7, IGF2, IGF2R, CXCR4, or CXCR2) can be the top 15% expression cutoff. The cutoff percentile can be the top 20% cutoff. The cutoff percentile can be the top 25% cutoff. The cutoff percentile can be the top 30% cutoff. The cutoff percentile can be the top 35% cutoff. The cutoff percentile can be the top 40% cutoff. The cutoff percentile can be the top 45% cutoff. The cutoff percentile can be the top 50% cutoff. For example, the reference expression level of CXCL12 can be a cutoff percentile of the expression in a population of subjects having the same type of tumor. The cutoff percentile can be the top 10%, 15%, 20%, 25%, 30%, 35%, 40%, 45%, or 50% cutoff of the expression level of CXCL12 in a population of subjects having the same type of tumor. For another example, the reference expression level of IGFBP7 can be a cutoff percentile of the expression in a population of subjects having the same type of tumor. The cutoff percentile can be the top 10%, 15%, 20%, 25%, 30%, 35%, 40%, 45%, or 50% cutoff of the expression level of IGFBP7 in a population of subjects having the same type of tumor.

In some embodiments, the reference expression level of a gene (e.g. CXCL12, IGF1, IGFBP7, IGF2, IGF2R, CXCR4, or CXCR2) for a particular cancer can be the bottom 10%, 15%, 20%, 25%, 30%, 35%, 40%, 45%, or 50% cutoff of the expression in a population of subjects having the same type of tumor. The cutoff percentile can be the bottom 10% cutoff. The cutoff percentile can be the bottom 15% expression cutoff. The cutoff percentile can be the bottom 20% cutoff. The cutoff percentile can be the bottom 25% cutoff. The cutoff percentile can be the bottom 30% cutoff. The cutoff percentile can be the bottom 35% cutoff. The cutoff percentile can be the bottom 40% cutoff. The cutoff percentile can be the bottom 45% cutoff. The cutoff percentile can be the bottom 50% cutoff. For example, the reference expression level of IGF1 for AML patients can be the bottom 30% cutoff of IGF1 expression in a population of AML patient.

In some embodiments, the reference expression level can be determined by a person of ordinary skill in the art through, for example, statistical analysis of the expression levels of the gene in samples from a clinical cohort.

The term “reference ratio” as used herein in connection with the expression levels of two or more genes refers to a ratio predetermined by a person of ordinary skill in the art that can be used to determine the significance of the ratio of the levels of these genes in a sample. The sample can be a cell, a group of cells, or a tissue. For example, a reference ratio of CXCL12 expression to CXCR4 expression can be a predetermined ratio of CXCL12 expression to CXCR4 expression. The reference ratio of the expression levels of two or more genes can be the median ratio of expression levels of these genes in a population of subjects. For example, a reference ratio of CXCL12 expression to CXCR4 expression can be the median ratio in a healthy population. For another example, a reference ratio of CXCL12 expression to CXCR4 expression can be the median ratio in a population of patients having the same type of tumor. The reference ratio can also be a cutoff percentile of the expression ratio in a population of subjects having the same type of tumor. The cutoff percentile can be the top 10%, 15%, 20%, 25%, 30%, 35%, 40%, 45%, or 50% cutoff. The cutoff percentile can be the top 10% cutoff. The cutoff percentile can be the top 15% expression cutoff. The cutoff percentile can be the top 20% cutoff. The cutoff percentile can be the top 25% cutoff. The cutoff percentile can be the top 30% cutoff. The cutoff percentile can be the top 35% cutoff. The cutoff percentile can be the top 40% cutoff. The cutoff percentile can be the top 45% cutoff. The cutoff percentile can be the top 50% cutoff. For example, the reference expression ratio for pancreatic cancer patients can be the top 30% cutoff of the ratio of CXCL12 expression to CXCR4 expression in a population of cancer patient. A reference ratio can also be a cut-off value determined by a person of ordinary skill in the art through, for example, statistical analysis of ratios of expression levels of the two genes in various sample cell populations. In certain embodiments, the reference ratio is 1/10, 1/9, 1/8, 1/7, 1/6, 1/5, 1/4, 1/3, 1/2, 1, 2, 3, 4, 5, 6, 7, 8, 9, 10, 15 or 20. In some embodiments, the reference ratio is 1/10. In some embodiments, the reference ratio is 1/9. In some embodiments, the reference ratio is 1/8. In some embodiments, the reference ratio is 1/7. In some embodiments, the reference ratio is 1/6. In some embodiments, the reference ratio is 1/5. In some embodiments, the reference ratio is 1/4. In some embodiments, the reference ratio is 1/3. In some embodiments, the reference ratio is 1/2. In some embodiments, the reference ratio is 1. In some embodiments, the reference ratio is 2. In some embodiments, the reference ratio is 3. In some embodiments, the reference ratio is 4. In some embodiments, the reference ratio is 5. In some embodiments, the reference ratio is 6. In some embodiments, the reference ratio is 7. In some embodiments, the reference ratio is 8. In some embodiments, the reference ratio is 9. In some embodiments, the reference ratio is 10. In some embodiments, the reference ratio is 15. In some embodiments, the reference ratio is 20.

As a person of ordinary skill in the art would understand, the reference expression level of a gene or the reference ratio between expression levels of two genes can also be determined based on statistical analysis of data from previous clinical trials, including outcome of a group of patients, namely, the patients' responsiveness to an FTI treatment, as well as the expression levels of the gene or ratio of expression levels between genes of the group of patients. A number of statistical methods are well known in the art to determine the reference level (or the “cut-off value”) of one or more genes when used to predict the responsiveness of a patient to a particular treatment, or to stratify patients for a particular treatment.

One method of the invention includes analyzing expression profiles for genes identified herein that distinguish responder from non-responder to determine the reference expression level for one or more genes. Comparisons between responders and non-responders can be performed using the Mann-Whitney U-test, Chi-square test, or Fisher's Exact test. Analysis of descriptive statistics and comparisons can be performed using SigmaStat Software (Systat Software, Inc., San Jose, Calif., USA).

In some embodiments, a classification and regression tree (CART) analysis can be adopted to determine the reference level. CART analysis is based on a binary recursive partitioning algorithm and allows for the discovery of complex predictor variable interactions that may not be apparent with more traditional methods, such as multiple linear regression. Binary recursive partitioning refers to the analysis that is: 1) binary, meaning there were two possible outcome variables, namely “responders” and “non-responders,” with the effect of splitting patients into 2 groups; 2) recursive, meaning the analysis can be performed multiple times; and 3) partitioned, meaning the entire data set can be split into sections. This analysis also has the ability to eliminate predictor variables with poor performance. The classification tree can be built using Salford Predictive Modeler v6.6 (Salford Systems, San Diego, Calif., USA).

Receiver Operator Characteristic (ROC) analysis can be utilized to determine the reference expression level, or reference expression ratio, or test the overall predictive value of individual genes and/or multiple genes. A review of the ROC analysis can be found in Soreide, J Clin Pathol 10.1136 (2008), which is hereby incorporated by reference in its entirety.

The reference level can be determined from the ROC curve of the training set to ensure both high sensitivity and high specificity. The performances of the predictors with different numbers of genes can be assessed based on misclassification error rate, sensitivity, specificity, p values measuring the separation of Kaplan-Meier curves of the two predicted groups.

The Top Scoring Pair (TSP) algorithm first introduced by Geman et al. (2004) can be used. In essence, the algorithm ranks all the gene pairs (genes i and j) based on the absolute difference (Dij) in the frequency of event where gene i has higher expression value than gene j in samples among class C1 to C2. In the cases of there are multiple top scoring pairs (all sharing the same Dij), the top pair by a secondary rank score that measures the magnitude to which inversions of gene expression levels occur from one class to the other within a pair of genes is selected. The top pair with highest frequency of absolute Dij>2 fold in all samples will be selected as candidate pair. The candidate pair can then be assessed in an independent testing data set. Leave-one-out cross validation (LOOCV) can be carried out in the training data set to evaluate how the algorithm perform. The performances of the predictors can be assessed based on maximum misclassification error rate. All the statistical analyses can be done using R (R Development Core Team, 2006).

Clinically reportable range (CRR) is the range of analyte values that a method can measure, allowing for specimen dilution, concentration, or other pretreatment used to extend the direct analytical measurement range. As provided in the Basic Methods Validation by Dr. Westgard, the experiment to be performed is often called a “linearity experiment,” though there technically is no requirement that a method provide a linear response unless two-point calibration is being used. This range can also be referred as the “linear range,” “analytical range,” or “working range” for a method.

The reportable range is assessed by inspection of the linearity graph. That inspection can involve manually drawing the best straight line through the linear portion of the points, drawing a point-to-point line through all the points then comparing with the best straight line, or fitting a regression line through the points in the linear range. There are more complicated statistical calculations that are recommended in some guidelines, such as Clinical Laboratory Standards Institute (CLSI)'s EP-6 protocol for evaluating the linearity of analytical methods. But it is commonly accepted that the reportable range can be adequately determined from a “visual” assessment, i.e., by manually drawing the best straight line that fits the lowest points in the series. The Clinical Laboratory Standards Institute (CLSI) recommends a minimum of at least 4- preferably 5-different levels of concentrations. More than 5 can be used, particularly if the upper limit of reportable range needs to be maximized, but 5 levels are convenient and almost always sufficient.

A reference interval is typically established by assaying specimens that are obtained from individuals that meet carefully defined criteria (reference sample group). Protocols such as those of the International Federation of Clinical Chemistry (IFCC) Expert Panel on Theory of Reference Values and the CLSI delineate comprehensive systematic processes that use carefully selected reference sample groups to establish reference intervals. These protocols typically need a minimum of 120 reference individuals for each group (or subgroup) that needs to be characterized.

The CLSI Approved Guideline C28-A2 describes different ways for a laboratory to validate the transference of established reference intervals to the individual laboratory that includes 1. Divine judgment, wherein the laboratory simply reviews the information submitted and subjectively verifies that the reference intervals are applicable to the adopting laboratory's patient population and test methods; 2. Verification with 20 samples, wherein experimental validation is performed by collecting and analyzing specimens from 20 individuals who represent the reference sample population; 3. Estimation with 60 samples, wherein an experimental validation is performed by collecting and analyzing specimens from 60 individuals who represent the reference sample population, and the actual reference interval is estimated and compared to the claimed or reported interval using a statistical formula comparing the means and standard deviations of the two populations; and 4. Calculation from comparative method, wherein one can adjust or correct the claimed or reported reference intervals on the basis of the observed methodological bias and the mathematical relationship demonstrated between the analytical methods being used.

A person of ordinary skill in the art would understand that the reference expression level of the genes disclosed herein as well as the reference ratios between two or more genes can be determined by one or more methods as provided herein or other methods known in the art.

### 2. COMBINATION THERAPY

2.1. FTI with IGF1R Pathway Inhibitor

As disclosed herein, IGF1R pathway mediates resistance to an FTI. Accordingly, disclosed herein are also methods of treating a cancer in a subject by administering to the subject a therapeutically effective amount of a FTI and a therapeutically effective amount of an inhibitor of IGF1R pathway. The inhibitor of IGF1R pathway sensitizes the cancer to FTI treatment and therefore achieves synergistic effect with FTI.

The inhibitor of IGF1R pathway can be an IGF1 inhibitor, an IGF1R inhibitor, a PI3K inhibitor, or an AKT inhibitor. In some embodiments, the inhibitor of IGF1R pathway can be an IGF1 inhibitor. In some embodiments, the inhibitor of IGF1R pathway is an IGF1R inhibitor. In some embodiments, the inhibitor of IGF1R pathway is a PI3K inhibitor. In some embodiments, the inhibitor of IGF1R pathway is an AKT inhibitor.

In some embodiments, provided herein are methods of treating a cancer in a subject by administering to the subject a therapeutically effective amount of a FTI and a therapeutically effective amount of an IGF1 inhibitor. The IGF1 inhibitor can be an anti-IGF1 antibody that blocks IGF1 from binding or activating IGF1R.

In some embodiments, provided herein are methods of treating a cancer in a subject by administering to the subject a therapeutically effective amount of a FTI and a therapeutically effective amount of an IGF1R inhibitor. The IGF1R inhibitor can be any anti-IGF1R antibody. The IGF1R inhibitor can be any IGF1R inhibitor known in the art, including, for example, A-923573, A-928605, A-947864, AEW-541, AG-1024, ANT-429, AVE1642, AZD-3463, BMS-754807, Ceritinib, FP-008, GSK-1904529A, GTx-134, IG01A-048, INT-231, juxtamembrane synthetic analogs, KW-2450, Linsitinib, LL-28, masoprocol, NVP-AEW541, NVP-ADW742, OSI-906, picropodophyllin, PL-225B, PNU-145156E, PQIP, XL-228, 1R-(E1)-(E1), AD-0027, ATL-1101, AVE-1642, BIIB-022, Cixutumumab, Dalotuzumab, Dusigitumab, Figitumumab, Ganitumab, h10H5, istiratumab, KM-1468, Lanreotide, m708.5, robatumumab, teprotumumab, and W-0101.

In some embodiments, provided herein are methods of treating a cancer in a subject by administering to the subject a therapeutically effective amount of a FTI and a therapeutically effective amount of an IGF1R inhibitor selected from the group consisting of: dalotuzumab, robatumumab, figitumumab, cixutumumab, ganitumab, AVE1642, OSI-906, NVP-AEW541 and NVP-ADW742.

In some embodiments, provided herein are methods of treating a cancer in a subject by administering to the subject a therapeutically effective amount of a FTI and a therapeutically effective amount of an PI3K inhibitor. The PI3K inhibitor can be any PI3K inhibitor known in the art, including, for example, 17 beta hydroxywortmannin analogs, 2-(4-piperazinyl)-substituted 4H-1-benzopyran-4-one derivatives, ACP-319, AEZS-129, AEZS-136, alirinetide, alpelisib, AMG-319, AMG-511, apitolisib, AQX-MN100, AQX-MN106, arsenic trioxide, AS-252424, AS-605240, ASN-003, Atu-027, AZD-6482, AZD-8154, AZD-8186, AZD-8835, BAG-956, BAY-1082439, BCN-004, BEBT-906, BEBT-908, BGT-226, bimiralisib, BMS-754807, BN-107, BR-101801, buparlisib, BVD-723, C-150, CAL-130, CAL-263, CC-115, CHR-4432, CL-27c, CLL-442, CLR-1401, CLR-1502, CLR-457, CMG-002, CNX-1351, copanlisib hydrochloride, CT-365, CT-732, CU-906, curcumin analogs, CYH-33, dactolisib, DCB-HDG2-57, dezapelisib, DS-7423, duvelisib, EC-0371, EC-0565, EM-101, EM-12, entospletinib, enzastaurin, everolimus, exemestane, EZN-4150, fimepinostat, FIM-X13, FP-208, FX-06, ganetespib, GAP-107B8, GDC-0084, GDC-0349, GDC-0941, gedatolisib, GS-548202, GS-599220, G5-9820, G5-9829, G5-9901, G5K-1059615, G5K-2126458, G5K-2292767, GSK-2636771, GSK-2702926A, GSK-418, GVK-01406, HEC-68498, HMPL-689, HNC-VP-L, HS-113, IBL-202, IBL-301, IC87114, idelalisib, IIIM-284/14, IIIM-873/15, IM-156, INCB-50465, INK-007, IP55 peptide, IPI-443, IPI-549, KA-2237, KAR-4141, KBP-7306, KD-06, LAS-191954, LAS-194223, leniolisib, LS-008, LY-294002, LY-3023414, MDN-088, ME-344, ME-401, MEN-1611, metformin butyrate, MLC-901, monepantel, MTX-211, MTX-216, nanolipolee-007, nemiralisib, neratinib, NV-128, NVP-BEZ235, OB-318, olcorolimus, oleandrin, ON-123300, ON-146 series, ONC-201, opaganib, OSI-027, OT-043, OXA-01, P-2281, P-6915, paclitaxel, panulisib, peptide H3, perifosine, PF-4691502, PF-4989216, phenytoin, PI-103, pictilisib, PIK-75, pilaralisib, PKI-179, PKI-402, PQR-311, PQR-312, PQR-316, PQR-340, PQR-370, PQR-401, PQR-514, PQR-530, PQR-SXX, PQR-620, PQR-6XX, puquitinib mesylate, PX-867, QLT-0447, rapamycin analog, rapamycin derivatives, Rapatar, RG-6114, ribociclib, ridaforolimus, rigosertib sodium, rilotumumab, risperidone, romidepsin, RP-5002, RP-5090, RP-6503, RV-1729, SAR-260301, SEL-403, seletalisib, serabelisib, SF-1126, SF-2535, SF-2558HA, silmitasertib, sirolimus, SKLB-JR02, SMI-4a, SN36093, solenopsin analogs, sonolisib, SRX-2523, SRX-2558, SRX-2626, SRX-3177, SRX-5000, ST-168, ST-182, sunitinib, SVP insulin, SVP-Rapamycin, TAFA-93, TAM-01, taselisib, TAT-N25 peptide, temsirolimus, tenalisib, TG-100-115, TGX-221, Triflorcas, ublituximab, umbralisib, Vanadis, VDC-597, VEL-015, voxtalisib, VS-5584, WJD-008, wortmannin, wumideji, WX-008, WX-037, WX-047, WXFL-10030390, X-339, X-370, X-414, X-480, XL-499, Y-31, YY-20394, YZJ-0673, and Z STK-474.

In some embodiments, provided herein are methods of treating a cancer in a subject by administering to the subject a therapeutically effective amount of a FTI and a therapeutically effective amount of an PI3K inhibitor selected from the group consisting of SF1126, TGX-221, PIK-75, PI-103, SN36093, IC87114, AS-252424, AS-605240, NVP-BEZ235, GDC-0941, ZSTK474, LY294002 and wortmannin.

In some embodiments, provided herein are methods of treating a cancer in a subject by administering to the subject a therapeutically effective amount of a FTI and a therapeutically effective amount of an AKT inhibitor. The AKT inhibitor can be any AKT inhibitor known in the art, including, for example, A-443654, Afuresertib, AMG-511, ARQ-751, AT-13148, AV-203, Capivasertib, CUDC-101, curcumin analog, deguelin, EM12, SK-2636771, GSK-690693, IMB-YH-8, INCB-047775, ipatasertib dihydrochloride, LY-2503029, LY-2780301, miransertib hydrochloride, MK-2206, MK-8156, nanoparticle MK-2206, OB-318, oxysterols, perifosine, PQR-401, PX-316, rigosertib sodium, SR-13668, SZ-685C, TAS-117, Triciribine, Triflorcas, Uprosertib, VEL-015, VLI-27, XL-418. In some embodiments, provided herein are methods of treating a cancer in a subject by administering to the subject a therapeutically effective amount of a FTI and a therapeutically effective amount of an AKT inhibitor selected from the group consisting of perifosine, SR13668, A-443654, triciribine, GSK690693, and deguelin.

The FTI used in combination with an inhibitor of the IGF1R pathway in cancer treatment can be any FTI known in the art. In some embodiments, the FTI can be tipifarnib, lonafarnib, arglabin, perrilyl alcohol, L778123, L739749, FTI-277, L744832, CP-609,754, R208176, AZD3409, or BMS-214662. In some embodiments, the FTI is lonafarnib. In some embodiments, the FTI is arglabin. In some embodiments, the FTI is perrilyl alcohol. In some embodiments, the FTI is L778123. In some embodiments, the FTI is L739749. In some embodiments, the FTI is FTI-277. In some embodiments, the FTI is L744832. In some embodiments, the FTI is CP-609. In some embodiments, the FTI is R208176. In some embodiments, the FTI is AZD3409. In some embodiments, the FTI is BMS-214662.

In some embodiments, the FTI is tipifarnib. Provided herein are also methods of treating a cancer in a subject by administering to the subject a therapeutically effective amount of tipifarnib and a therapeutically effective amount of an inhibitor of IGF1R pathway. The inhibitor of IGF1R pathway sensitizes the cancer to tipifarnib treatment and therefore achieves synergistic effect with tipifarnib. In some embodiments, the inhibitor of IGF1R pathway can be an IGF1 inhibitor. In some embodiments, the inhibitor of IGF1R pathway is an IGF1R inhibitor. In some embodiments, the inhibitor of IGF1R pathway is a PI3K inhibitor. In some embodiments, the inhibitor of IGF1R pathway is an AKT inhibitor.

In some embodiments, provided herein are methods of treating a cancer in a subject by administering to the subject a therapeutically effective amount of a tipifarnib and a therapeutically effective amount of an IGF1R inhibitor. The IGF1R inhibitor can be selected from the group consisting of: dalotuzumab, robatumumab, figitumumab, cixutumumab, ganitumab, AVE1642, OSI-906, NVP-AEW541 and NVP-ADW742.

In some embodiments, provided herein are methods of treating a cancer in a subject by administering to the subject a therapeutically effective amount of a tipifarnib and a therapeutically effective amount of an PI3K inhibitor. The PI3K inhibitor can be selected from the group consisting of SF1126, TGX-221, PIK-75, PI-103, SN36093, IC87114, AS-252424, AS-605240, NVP-BEZ235, GDC-0941, ZSTK474, LY294002 and wortmannin.

In some embodiments, provided herein are methods of treating a cancer in a subject by administering to the subject a therapeutically effective amount of a tipifarnib and a therapeutically effective amount of an AKT inhibitor. The AKT inhibitor can be selected from the group consisting of perifosine, SR13668, A-443654, triciribine phosphate monohydrate, GSK690693, and deguelin.

In some embodiments, the combination of an FTI and an inhibitor the IFG1R pathway can be used in treating a hematological cancer. In some embodiments, the hematological cancer is a myeloid hematological cancer. The myeloid hematological cancer can be selected from the group consisting of acute myeloid leukemia (AML), myeloproliferative neoplasm (MPN), myelodysplastic syndrome (MDS), chronic myelomonocytic leukemia (CMML) and chronic myeloid leukemia (CIVIL). In some embodiments, the hematological cancer is AML. In some embodiments, the hematological cancer is CMML. In some embodiments, the hematological cancer is MPN. In some embodiments, the hematological cancer is MDS.

In some embodiments, the hematological cancer is a lymphoid hematological cancer. The lymphoid hematological cancer can be selected from the group consisting of natural killer cell lymphoma (NK lymphoma), natural killer cell leukemia (NK leukemia), cutaneous T-Cell lymphoma (CTCL), and peripheral T-cell lymphoma (PTCL). In some embodiments, the hematological cancer is NK lymphoma. In some embodiments, the hematological cancer is NK leukemia. In some embodiments, the hematological cancer is CTCL. In some embodiments, the hematological cancer is PTCL.

In some embodiments, the combination of an FTI and an inhibitor the IFG1R pathway can be used in treating a solid tumor. The solid tumor can be pancreatic cancer, bladder cancer, breast cancer, gastric cancer, colorectal cancer, head and neck cancer, head and neck squamous cell carcinoma, mesothelioma, uveal melanoma, glioblastoma, adrenocortical carcinoma, esophageal cancer, melanoma, lung adenocarcinoma, lung squamous carcinoma, ovarian cancer, hepatocellular carcinoma, sarcoma, or prostate cancer. In some embodiments, the solid tumor is pancreatic cancer. In some embodiments, the pancreatic cancer is pancreatic ductal adenocarcinoma (PDAC). In some embodiments, the cancer is squamous cell carcinoma. In some embodiments, the solid tumor is bladder cancer. In some embodiments, the solid tumor is breast cancer. In some embodiments, the solid tumor is gastric cancer. In some embodiments, the solid tumor is colorectal cancer. In some embodiments, the solid tumor is head and neck cancer. In some embodiments, the solid tumor is mesothelioma. In some embodiments, the solid tumor is uveal melanoma. In some embodiments, the solid tumor is glioblastoma. In some embodiments, the solid tumor is adrenocortical carcinoma. In some embodiments, the solid tumor is adrenocortical carcinoma. In some embodiments, the solid tumor is esophageal cancer. In some embodiments, the solid tumor is melanoma. In some embodiments, the solid tumor is lung adenocarcinoma. In some embodiments, the solid tumor is prostate cancer. In some embodiments, the solid tumor is lung squamous carcinoma. In some embodiment, the solid tumor is head and neck squamous carcinoma. In some embodiments, the solid tumor is ovarian cancer. In some embodiments, the solid tumor is hepatocellular carcinoma. In some embodiments, the solid tumor is sarcoma.

Embodiments explicitly described herein are intended to exemplify and not limiting. The present disclose also includes all combinations and permutations of different FTI and inhibitors of the IGF1R inhibitor in the treatment of different types of cancer.

2.2. FTI with CXCR4 Inhibitor

Provided herein are combination therapies that include an FTI and an inhibitor of the CXCR4 pathway for cancer treatment. For example, in some embodiments, provided herein are methods of treating a cancer in a subject by administering to the subject a therapeutically effective FTI and a therapeutically effective amount of CXCR4 antagonist. The CXCR4 antagonists include, for example, Plerixafor, chloroquine, and hydroxychloroquine. Kim J et al., PLoS One, 2012; 7(2):e31004; Sorensen et al., Mol. Cancer Ther. 2014; 13(7):1758-71. Schmukler E et al., Oncotarget. 2014 Jan. 15; 5(1):173-84.

As disclosed herein, CXCR4 can drive sensitivity to an FTI. Accordingly, disclosed herein are also methods of treating a cancer in a subject by administering to the subject a therapeutically effective amount of a FTI and a therapeutically effective amount of an antagonist of CXCR4. The CXCR4 antagonist can be any CXCR4 antagonist known in the art, including for example AD-114, ALT-1188, AMD-070, AMD-3100, APH-0812, balixafortide, BKT-140, BKT-170, BL-8040, burixafor, CCRS receptor modulator, cefprozil, chloroquine, hydroxychloroquine, CTCE-0012, CX-549, DBPR-215, D-Lys3 GHRP-6, F-50067, filgrastim, GBV-4086, GMI-1359, KRH-1120, KRH-3166, KRH-3955, LY-2510924, LY-2624587, MEDI-3185, N15P peptide, ND-401, NSC-651016, ONO-7161, PF-06747143, plerixafor, POL-5551, PTX-9908, SDF-1 antibody, T-134, TIQ-15 analogs, Ulocuplumab, USL-311, VIR-5103, vMIP, vMIP-II, X4-136, X4P-001, or X4P-002. In some embodiments, provided herein are methods of treating a cancer in a subject by administering to the subject a therapeutically effective amount of a FTI and a therapeutically effective amount of an antagonist of CXCR4 selected from the group consisting of AMD-3100, BL-8040, chloroquine, and plerixafor.

The FTI used in combination with an inhibitor of the IGF1R pathway in cancer treatment can be any FTI known in the art. In some embodiments, the FTI can be tipifarnib, lonafarnib, arglabin, perrilyl alcohol, L778123, L739749, FTI-277, L744832, CP-609,754, R208176, AZD3409, or BMS-214662. In some embodiments, the FTI is lonafarnib. In some embodiments, the FTI is arglabin. In some embodiments, the FTI is perrilyl alcohol. In some embodiments, the FTI is L778123. In some embodiments, the FTI is L739749. In some embodiments, the FTI is FTI-277. In some embodiments, the FTI is L744832. In some embodiments, the FTI is CP-609. In some embodiments, the FTI is R208176. In some embodiments, the FTI is AZD3409. In some embodiments, the FTI is BMS-214662.

In some embodiments, the FTI is tipifarnib. Provided herein are also methods of treating a cancer in a subject by administering to the subject a therapeutically effective amount of tipifarnib and a therapeutically effective amount of an antagonist of CXCR4. The CXCR4 antagonist can be any CXCR4 antagonist known in the art, including for example AD-114, ALT-1188, AMD-070, AMD-3100, APH-0812, balixafortide, BKT-140, BKT-170, BL-8040, burixafor, CCR5 receptor modulator, cefprozil, chloroquine, hydroxychloroquine, CTCE-0012, CX-549, DBPR-215, D-Lys3 GHRP-6, F-50067, filgrastim, GBV-4086, GMI-1359, KRH-1120, KRH-3166, KRH-3955, LY-2510924, LY-2624587, MEDI-3185, N15P peptide, ND-401, NSC-651016, ONO-7161, PF-06747143, plerixafor, POL-5551, PTX-9908, SDF-1 antibody, T-134, TIQ-15 analogs, Ulocuplumab, USL-311, VIR-5103, vMIP, vMIP-II, X4-136, X4P-001, or X4P-002. In some embodiments, provided herein are methods of treating a cancer in a subject by administering to the subject a therapeutically effective amount of tipifarnib and a therapeutically effective amount of an antagonist of CXCR4 selected from the group consisting of AMD-3100, BL-8040, chloroquine, and plerixafor.

In some embodiments, the combination of an FTI and an antagonist of CXCR4 can be used in treating a hematological cancer. In some embodiments, the hematological cancer is a myeloid hematological cancer. The myeloid hematological cancer can be selected from the group consisting of acute myeloid leukemia (AML), myeloproliferative neoplasm (MPN), myelodysplastic syndrome (MDS), chronic myelomonocytic leukemia (CMML) and chronic myeloid leukemia (CML). In some embodiments, the hematological cancer is AML. In some embodiments, the hematological cancer is CMML. In some embodiments, the hematological cancer is MPN. In some embodiments, the hematological cancer is MDS.

In some embodiments, the hematological cancer is a lymphoid hematological cancer. The lymphoid hematological cancer can be selected from the group consisting of natural killer cell lymphoma (NK lymphoma), natural killer cell leukemia (NK leukemia), cutaneous T-Cell lymphoma (CTCL), and peripheral T-cell lymphoma (PTCL). In some embodiments, the hematological cancer is NK lymphoma. In some embodiments, the hematological cancer is NK leukemia. In some embodiments, the hematological cancer is CTCL. In some embodiments, the hematological cancer is PTCL.

In some embodiments, the combination of an FTI and an antagonist of CXCR4 can be used in treating a solid tumor. The solid tumor can be pancreatic cancer, bladder cancer, breast cancer, gastric cancer, colorectal cancer, head and neck cancer, head and neck squamous cell carcinoma, mesothelioma, uveal melanoma, glioblastoma, adrenocortical carcinoma, esophageal cancer, melanoma, lung adenocarcinoma, lung squamous carcinoma, ovarian cancer, hepatocellular carcinoma, sarcoma, or prostate cancer. In some embodiments, the solid tumor is pancreatic cancer. In some embodiments, the pancreatic cancer is pancreatic ductal adenocarcinoma (PDAC). In some embodiments, the cancer is squamous cell carcinoma. In some embodiments, the solid tumor is bladder cancer. In some embodiments, the solid tumor is breast cancer. In some embodiments, the solid tumor is gastric cancer. In some embodiments, the solid tumor is colorectal cancer. In some embodiments, the solid tumor is head and neck cancer. In some embodiments, the solid tumor is mesothelioma. In some embodiments, the solid tumor is uveal melanoma. In some embodiments, the solid tumor is glioblastoma. In some embodiments, the solid tumor is adrenocortical carcinoma. In some embodiments, the solid tumor is In some embodiments, the solid tumor is adrenocortical carcinoma. In some embodiments, the solid tumor is esophageal cancer. In some embodiments, the solid tumor is melanoma. In some embodiments, the solid tumor is lung adenocarcinoma. In some embodiments, the solid tumor is prostate cancer. In some embodiments, the solid tumor is lung squamous carcinoma. In some embodiments, the solid tumor is head and neck squamous cell carcinoma. In some embodiments, the solid tumor is ovarian cancer. In some embodiments, the solid tumor is hepatocellular carcinoma. In some embodiments, the solid tumor is sarcoma.

Embodiments explicitly described herein are intended to exemplify and not limiting. The present disclose also includes all combinations and permutations of using different FTI and antagonists of CXCR4 in the treatment of different types of cancer.

### 2.3. Additional Therapies

The FTI treatment, alone or in combination with either an inhibitor of IGF1R pathway or a CXCR4 antagonist as described herein, can also be used in combination with additional therapies in selectively treating cancer patients. The FTI can be any FTI as described herein or otherwise known in the art. In some embodiments, the FTI can be tipifarnib, lonafarnib, arglabin, perrilyl alcohol, L778123, L739749, FTI-277, L744832, CP-609,754, R208176, AZD3409, or BMS-214662. In some embodiments, the FTI is tipifarnib.

In some embodiments, the FTI treatment is administered in combination with radiotherapy, or radiation therapy. Radiotherapy includes using y-rays, X-rays, and/or the directed delivery of radioisotopes to tumor cells. Other forms of DNA damaging factors are also contemplated, such as microwaves, proton beam irradiation (U.S. Pat. Nos. 5,760,395 and 4,870,287; all of which are hereby incorporated by references in their entireties), and UV-irradiation. It is most likely that all of these factors affect a broad range of damage on DNA, on the precursors of DNA, on the replication and repair of DNA, and on the assembly and maintenance of chromosomes.

In some embodiments, a therapeutically effective amount of the pharmaceutical composition having an FTI is administered that effectively sensitizes a tumor in a host to irradiation. (U.S. Pat. No. 6545020, which is hereby incorporated by reference in its entirety). Irradiation can be ionizing radiation and in particular gamma radiation. In some embodiments, the gamma radiation is emitted by linear accelerators or by radionuclides. The irradiation of the tumor by radionuclides can be external or internal.

Irradiation can also be X-ray radiation. Dosage ranges for X-rays range from daily doses of 50 to 200 roentgens for prolonged periods of time (3 to 4 wk), to single doses of 2000 to 6000 roentgens. Dosage ranges for radioisotopes vary widely, and depend on the half-life of the isotope, the strength and type of radiation emitted, and the uptake by the neoplastic cells.

In some embodiments, the administration of the pharmaceutical composition commences up to one month, in particular up to 10 days or a week, before the irradiation of the tumor. Additionally, irradiation of the tumor is fractionated the administration of the pharmaceutical composition is maintained in the interval between the first and the last irradiation session.

The amount of FTI, the dose of irradiation and the intermittence of the irradiation doses will depend on a series of parameters such as the type of tumor, its location, the patients' reaction to chemo- or radiotherapy and ultimately is for the physician and radiologists to determine in each individual case. In some embodiments, the FTI is administered before the administration of a radiation therapy. In some embodiments, the FTI is administered concurrently with a radiation therapy. In some embodiments, the FTI is administered after the administration of a radiation therapy. In some embodiments, the FTI is tipifarnib.

In some embodiments, the methods provided herein further include administering a therapeutically effective amount of an additional active agent or a support care therapy. The additional active agent can be a chemotherapeutic agent. A chemotherapeutic agent or drug can be categorized by its mode of activity within a cell, for example, whether and at what stage they affect the cell cycle. Alternatively, an agent can be characterized based on its ability to directly cross-link DNA, to intercalate into DNA, or to induce chromosomal and mitotic aberrations by affecting nucleic acid synthesis. The FTI can be administered before the administration of an additional active agent. The FTI can be administered concurrently with an additional active agent. The FTI can be administered after the administration of an active agent.

Examples of chemotherapeutic agents include alkylating agents, such as thiotepa and cyclosphosphamide; alkyl sulfonates, such as busulfan, improsulfan, and piposulfan; aziridines, such as benzodopa, carboquone, meturedopa, and uredopa; ethylenimines and methylamelamines, including altretamine, triethylenemelamine, trietylenephosphoramide, triethiylenethiophosphoramide, and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards, such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, and uracil mustard; nitrosureas, such as carmustine, chlorozotocin, fotemustine, lomustine, nimustine, and ranimnustine; antibiotics, such as the enediyne antibiotics (e.g., calicheamicin, especially calicheamicin gammall and calicheamicin omegaI1); dynemicin, including dynemicin A; bisphosphonates, such as clodronate; an esperamicin; as well as neocarzinostatin chromophore and related chromoprotein enediyne antiobiotic chromophores, aclacinomysins, actinomycin, anthramycin, azaserine, bleomycins, cactinomycin, carabicin, carminomycin, carzinophilin, chromomycinis, dactinomycin, daunorubicin, detorubicin, 6-diazo-5-oxo-L-norleucine, doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins, such as mitomycin C, mycophenolic acid, nogalarnycin, olivomycins, peplomycin, potfiromycin, puromycin, quelamycin, rodorubicin, streptonigrin, streptozocin, tubercidin, ubenimex, zinostatin, and zorubicin; anti-metabolites, such as methotrexate and 5-fluorouracil (5-FU); folic acid analogues, such as denopterin, pteropterin, and trimetrexate; purine analogs, such as fludarabine, 6-mercaptopurine, thiamiprine, and thioguanine; pyrimidine analogs, such as ancitabine, azacitidine, 6-azauridine, carmofur, cytarabine, dideoxyuridine, doxifluridine, enocitabine, and floxuridine; androgens, such as calusterone, dromostanolone propionate, epitiostanol, mepitiostane, and testolactone; anti-adrenals, such as mitotane and trilostane; folic acid replenisher, such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids, such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSKpolysaccharide complex; razoxane; rhizoxin; sizofiran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; taxoids, e.g., paclitaxel and docetaxel gemcitabine; 6-thioguanine; mercaptopurine; platinum coordination complexes, such as cisplatin, oxaliplatin, and carboplatin; vinblastine; platinum; etoposide (VP-16); ifosfamide; mitoxantrone; vincristine; vinorelbine; novantrone; teniposide; edatrexate; daunomycin; aminopterin; xeloda; ibandronate; irinotecan (e.g., CPT-11); topoisomerase inhibitor RFS 2000; difluorometlhylornithine (DMFO); retinoids, such as retinoic acid; capecitabine; carboplatin, procarbazine, plicomycin, gemcitabine, navelbine, transplatinum, and pharmaceutically acceptable salts, acids, or derivatives of any of the above.

In some embodiments, the additional active agent to be administered to the subject is capecitabine. In certain embodiments, capecitabine can be administered to the subject at a dosage of 100 mg/m2, 200 mg/m2, 300 mg/m2, 400 mg/m2, 500 mg/m2, 600 mg/m2, 700 mg/m2, 800 mg/m2, 900 mg/m2, 1,000 mg/m2, 1100 mg/m2, 1200 mg/m2, 1300 mg/m2, 1400 mg/m2, or 1500 mg/m2. In certain embodiments, capecitabine is administered daily. In certain embodiments, the capecitabine is administered twice daily.

A treatment cycle can have different length. In some embodiments, a treatment cycle can be one week, 2 weeks, 3 weeks, 4 weeks, 5 weeks, 6 weeks, 7 weeks, 8 weeks, 3 months, 4 months, 5 months, 6 months, 7 months, 8 months, 9 months, 10 months, 11 months, or 12 months. In some embodiments, a treatment cycle is 3 weeks. In some embodiments, a treatment cycle is 4 weeks. A treatment cycle can have intermittent schedule. In some embodiments, a 3-week treatment cycle can have 7-day dosing, followed by 14-day rest. In some embodiments, a 3-week treatment cycles can have 14-day dosing followed by 7-day rest. In some embodiment, a 4 week treatment cycle can have 7 day dosing, followed by 21-day rest. In some embodiment, a 4 week treatment cycle can have 14 day dosing, followed by 14-day rest. In some embodiment, a 4 week treatment cycle can have 21 day dosing, followed by 7-day rest. In some embodiment, a 4 week treatment cycle can have dosing on days 1-7 and 15-21, and rest on days 8-14 and 22-28.

In some embodiments, the capecitabine can be administered for at least one treatment cycle. In some embodiments, the capecitabine can be administered for at least two, at least three, at least four, at least five, at least six, at least seven, at least eight, at least nine, at least ten, at least eleven, or at least twelve treatment cycles. In some embodiments, the capecitabine can be administered for at least three treatment cycles.

In some embodiments, the capecitabine is administered for up to two weeks. In some embodiments, the capecitabine is administered for up to three weeks, up to one month, up to two months, up to three months, up to four months, up to five months, up to six months, up to seven months, up to eight months, up to nine months, up to ten months, up to eleven months, or up to twlve months. In some embodiments, the capecitabine treatment therapy can be maintained for at least 6 months beyond the start of the response.

In some embodiments, the capecitabine is administered daily for 1 (days 1-7) of out of 3 weeks in repeated 3 week cycles. In some embodiments, the capecitabine is administered daily for 2 (days 1-14) of out of 3 weeks in repeated 3 week cycles. In some embodiments, the capecitabine is administered at a dose of 1000 mg/m2 b.i.d. orally for 1(days 1-7) of out of 3 weeks in repeated 3 week cycles. In some embodiments, the capecitabine is administered at a dose of 1000 mg/m2 b.i.d. orally for 2 (days 1-14) of out of 3 weeks in repeated 3 week cycles. In some embodiments, the capecitabine is administered daily for 3 of out of 4 weeks in repeated 4 week cycles. In some embodiments, the capecitabine is administered daily in alternate weeks (one week on, one week off) in repeated 4 week cycles. In some embodiments, the capecitabine is administered at a dose of 1000 mg/m2 b.i.d. orally for 3 of out of 4 weeks in repeated 4 week cycles.

The additional active agents can be large molecules (e.g., proteins) or small molecules (e.g., synthetic inorganic, organometallic, or organic molecules). In some embodiments, the additional active agent is a DNA-hypomethylating agent, an alkylating agent, a topoisomerase inhibitor, a CDK inhibitor, or a therapeutic antibody that specifically binds to a cancer antigen. The additional active agent can be also a hematopoietic growth factor, a cytokine, an antibiotic, a cox-2 inhibitor, an immunomodulatory agent, anti-thymocyte globulin, an immunosuppressive agent, corticosteroid or a pharmacologically active mutant or derivative thereof.

In some embodiments, the additional therapy is a chemotherapy, such as cisplatin, 5-FU, carboplatin, paclitaxel, or platinum-based doublet (e.g., cisplatin/5-FU or carboplatin/paclitaxel). In some embodiments, the additional therapy is taxanes and/or methotrexate. In some embodiments, the additional therapy can be selected from those targeting PI3K pathway: BKM120 (buparlisib), BYL719, Temsirolimus, Rigosertib; those targeting MET pathway: Tivantinib, Ficlatuzumab; those targeting the HER3 pathway, Patritumab; those targeting FGFR pathway: BGJ398; those targeting CDK4/6-cell cycle pathway: Palbociclib, LEE011, abemaciclib, and ribociclib; RTK inhibitor: Anlotinib; AKT inhibitors: MK2206, GSK2110183, and GSK2141795; and chemotherapy: Oral Azacitidine. In some embodiments, the additional therapy is an immunotherapy, such as anti-PD1 antibodies, anti-PDL1 antibodies, or ant-CTLA-4 antibodies. In some embodiments, the additional therapy is a taxane.

In some embodiments, the additional active agent is an EGFR inhibitor. The EGFR inhibitor can be an anti-EGFR antibody, for example, geFTIinib, erlotinib, neratinib, lapatinib, vandetanib, cetuximab, necitumumab, osimertinib, or panitumumab. The FTI can be administered before the administration of an EGFR inhibitor. The FTI can be administered concurrently with an EGFR inhibitor. The FTI can be administered after the administration of an EGFR inhibitor. In some embodiments, the additional active agent is a DNA hypomethylating agent, such as a cytidine analog (e.g., azacitidine) or a 5-azadeoxycytidine (e.g. decitabine). In some embodiments, the additional active agent is a cytoreductive agent, including but not limited to Induction, Topotecan, Hydrea, PO Etoposide, Lenalidomide, LDAC, and Thioguanine. In some embodiments, the additional active agent is Mitoxantrone, Etoposide, Cytarabine, or Valspodar. In some embodiment, the additional active agent is Mitoxantrone plus Valspodar, Etoposide plus Valspodar, or Cytarabine plus Valspodar. In some embodiment, the additional active agent is idarubicin, fludarabine, topotecan, or ara-C. In some other embodiments, the additional active agent is idarubicin plus ara-C, fludarabine plus ara-C, mitoxantrone plus ara-C, or topotecan plus ara-C. In some embodiments, the additional active agent is a quinine. Other combinations of the agents specified above can be used, and the dosages can be determined by the physician.

Treatments as described herein or otherwise available in the art can be used in combination with the FTI treatment. For example, drugs that can be used in combination with the FTI include belinostat (Beleodaq) and pralatrexate (Folotyn), marketed by Spectrum Pharmaceuticals, romidepsin (Istodax®), marketed by Celgene, and brentuximab vedotin (Adcetris®), marketed by Seattle Genetics; azacytidine (Vidaza®) and lenalidomide (Revlimid®), marketed by Celgene, and decitabine (Dacogen®) marketed by Otsuka and Johnson & Johnson; vandetanib (Caprelsa®), Bayer's sorafenib (Nexavar®), Exelixis' cabozantinib (Cometriq®) and Eisai's lenvatinib (Lenvima®). Non-cytotoxic therapies such as pralatrexate (Folotyn®), romidepsin (Istodax®) and belinostat (Beleodaq®) can also be used in combination with the FTI treatment. In some embodiments, the additional active agent is a DNA-hypomethylating agent. In some embodiments, the additional active agent is cytarabine, daurubicin, idarubicin, or gentuzumab, or ozogamicin. In some embodiments, the additional active agent is a DNA-hypomethylating agent, such as azacitidine or decitabine.

In some embodiments, the additional active agent is an immunotherapy agent. In some embodiments, the additional active agent is anti-PD1 antibody. In some embodiments, the additional active agent is an anti-PDL1 antibody. In some embodiments, the additional active agent is an anti-CTLA-4 antibody.

The additional active agent or therapy used in combination with an FTI can be administered before, at the same time, or after the FTI treatment. In some embodiments, the additional active agent or therapy used in combination with an FTI can be administered before the FTI treatment. In some embodiments, the additional active agent or therapy used in combination with an FTI can be administered at the same time as FTI treatment. In some embodiments, the additional active agent or therapy used in combination with an FTI can be administered after the FTI treatment.

In some embodiments, the FTI treatment is administered in combination with a bone marrow transplant. In some embodiments, the FTI is administered before the administration of a bone marrow transplant. In some embodiments, the FTI is administered concurrently with a bone marrow transplant. In some embodiments, the FTI is administered after the administration of a bone marrow transplant.

In some embodiments, the FTI treatment is administered in combination with a stem cell transplant. In some embodiments, the FTI is administered before the administration of a stem cell transplant. In some embodiments, the FTI is administered concurrently with a stem cell transplant. In some embodiments, the FTI is administered after the administration of a stem cell transplant.

A person of ordinary skill in the art would understand that the methods described herein include using any permutation or combination of the specific FTI, formulation, dosing regimen, additional therapy to treat a subject described herein.

### 3. PHARMACEUTICAL COMPOSITIONS

In some embodiments, provided herein is a method of treating a subject with an FTI or a pharmaceutical composition having an FTI. The pharmaceutical compositions provided herein contain therapeutically effective amounts of an FTI and a pharmaceutically acceptable carrier, diluent or excipient. In some embodiments, the FTI is tipifarnib; lonafarnib (also known as SCH-66336); arglabin; perrilyl alcohol; CP-609,754, BMS 214662; L778123; L744832; L739749; R208176; AZD3409; or FTI-277. In some embodiments, the FTI is tipifarnib.

Provided herein are also pharmaceutical compositions having therapeutically effective amounts of an FTI and an inhibitor of an IGF1R pathway and a pharmaceutically acceptable carrier, diluent or excipient. In some embodiments, the FTI is tipifarnib; lonafarnib (also known as SCH-66336); arglabin; perrilyl alcohol; CP-609,754, BMS 214662; L778123; L744832; L739749; R208176; AZD3409; or FTI-277. Provided herein are also pharmaceutical compositions having therapeutically effective amounts of tipifarnib and an inhibitor of an IGF1R pathway and a pharmaceutically acceptable carrier, diluent or excipient.

The inhibitor of IGF1R pathway can be an IGF1 inhibitor, an IGF1R inhibitor, a PI3K inhibitor, or an AKT inhibitor. In some embodiments, the inhibitor of IGF1R pathway can be an IGF1 inhibitor. In some embodiments, the inhibitor of IGF1R pathway is an IGF1R inhibitor. In some embodiments, the inhibitor of IGF1R pathway is a PI3K inhibitor. In some embodiments, the inhibitor of IGF1R pathway is an AKT inhibitor.

In some embodiments, provided herein are pharmaceutical compositions having therapeutically effective amounts of an FTI and an IGF1 inhibitor, and a pharmaceutically acceptable carrier, diluent or excipient. In some embodiments, the IGF1 inhibitor can be an anti-IGF1 antibody that blocks IGF1 from binding or activing IGF1R.

In some embodiments, provided herein are pharmaceutical compositions having therapeutically effective amounts of an FTI and an IGF1R inhibitor, and a pharmaceutically acceptable carrier, diluent or excipient. The IGF1R inhibitor can be an anti-IGF1R antibody. The IGF1R inhibitor can be any IGF1R inhibitor known in the art, including, for example, A-923573, A-928605, A-947864, AEW-541, AG-1024, ANT-429, AVE1642, AZD-3463, BMS-754807, Ceritinib, FP-008, GSK-1904529A, GTx-134, IG01A-048, INT-231, juxtamembrane synthetic analogs, KW-2450, Linsitinib, LL-28, masoprocol, NVP-AEW541, NVP-ADW742, OSI-906, picropodophyllin, PL-225B, PNU-145156E, PQIP, XL-228, 1R-(E1)-(E1), AD-0027, ATL-1101, AVE-1642, BIIB-022, Cixutumumab, Dalotuzumab, Dusigitumab, Figitumumab, Ganitumab, h10H5, istiratumab, KM-1468, Lanreotide, m708.5, robatumumab, teprotumumab, and W-0101.

In some embodiments, provided herein are pharmaceutical compositions having therapeutically effective amounts of an FTI and an IGF1R inhibitor, and a pharmaceutically acceptable carrier, diluent or excipient, wherein the IGF1R inhibitor is selected from the group consisting of: dalotuzumab, robatumumab, figitumumab, cixutumumab, ganitumab, AVE1642, OSI-906, NVP-AEW541 and NVP-ADW742.

In some embodiments, provided herein are pharmaceutical compositions having therapeutically effective amounts of an FTI and a PI3K inhibitor, and a pharmaceutically acceptable carrier, diluent or excipient. The PI3K inhibitor can be any PI3K inhibitor known in the art, including, for example, 17 beta hydroxywortmannin analogs, 2-(4-piperazinyl)-substituted 4H-1-benzopyran-4-one derivatives, ACP-319, AEZS-129, AEZS-136, alirinetide, alpelisib (BYL-719), AMG-319, AMG-511, apitolisib, AQX-MN100, AQX-MN106, arsenic trioxide, AS-252424, AS-605240, ASN-003, Atu-027, AZD-6482, AZD-8154, AZD-8186, AZD-8835, BAG-956, BAY-1082439, BCN-004, BEBT-906, BEBT-908, BGT-226, bimiralisib, BMS-754807, BN-107, BR-101801, buparlisib, BVD-723, C-150, CAL-130, CAL-263, CC-115, CHR-4432, CL-27c, CLL-442, CLR-1401, CLR-1502, CLR-457, CMG-002, CNX-1351, copanlisib hydrochloride, CT-365, CT-732, CU-906, curcumin analogs, CYH-33, dactolisib, DCB-HDG2-57, dezapelisib, DS-7423, duvelisib, EC-0371, EC-0565, EM-101, EM-12, entospletinib, enzastaurin, everolimus, exemestane, EZN-4150, fimepinostat, FIM-X13, FP-208, FX-06, ganetespib, GAP-107B8, GDC-0084, GDC-0349, GDC-0941, gedatolisib, GS-548202, GS-599220, GS-9820, GS-9829, GS-9901, GSK-1059615, GSK-2126458, GSK-2292767, GSK-2636771, GSK-2702926A, GSK-418, GVK-01406, HEC-68498, HMPL-689, HNC-VP-L, HS-113, IBL-202, IBL-301, IC87114, idealisib, IIIM-284/14, IIIM-873/15, IM-156, INCB-50465, INK-007, IP55 peptide, IPI-443, IPI-549, KA-2237, KAR-4141, KBP-7306, KD-06, LAS-191954, LAS-194223, leniolisib, LS-008, LY-294002, LY-3023414, MDN-088, ME-344, ME-401, MEN-1611, metformin butyrate, MLC-901, monepantel, MTX-211, MTX-216, nanolipolee-007, nemiralisib, neratinib, NV-128, NVP-BEZ235, OB-318, olcorolimus, oleandrin, ON-123300, ON-146 series, ONC-201, opaganib, OSI-027, OT-043, OXA-01, P-2281, P-6915, paclitaxel, panulisib, peptide H3, perifosine, PF-4691502, PF-4989216, phenytoin, PI-103, pictilisib, PIK-75, pilaralisib, PKI-179, PKI-402, PQR-311, PQR-312, PQR-316, PQR-340, PQR-370, PQR-401, PQR-514, PQR-530, PQR-SXX, PQR-620, PQR-6XX, puquitinib mesylate, PX-867, QLT-0447, rapamycin analog, rapamycin derivatives, Rapatar, RG-6114, ribociclib, ridaforolimus, rigosertib sodium, rilotumumab, risperidone, romidepsin, RP-5002, RP-5090, RP-6503, RV-1729, SAR-260301, SEL-403, seletalisib, serabelisib, SF-1126, SF-2535, SF-2558HA, silmitasertib, sirolimus, SKLB-JR02, SMI-4a, SN36093, solenopsin analogs, sonolisib, SRX-2523, SRX-2558, SRX-2626, SRX-3177, SRX-5000, ST-168, ST-182, sunitinib, SVP insulin, SVP-Rapamycin, TAFA-93, TAM-01, taselisib, TAT-N25 peptide, temsirolimus, tenalisib, TG-100-115, TGX-221, Triflorcas, ublituximab, umbralisib, Vanadis, VDC-597, VEL-015, voxtalisib, VS-5584, WJD-008, wortmannin, wumideji, WX-008, WX-037, WX-047, WXFL-10030390, X-339, X-370, X-414, X-480, XL-499, Y-31, YY-20394, YZJ-0673, and Z STK-474.

In some embodiments, provided herein are pharmaceutical compositions having therapeutically effective amounts of an FTI and a PI3K inhibitor, and a pharmaceutically acceptable carrier, diluent or excipient, wherein the PI3K inhibitor is selected from the group consisting of SF1126, TGX-221, PIK-75, PI-103, SN36093, IC87114, AS-252424, AS-605240, NVP-BEZ235, GDC-0941, ZSTK474, LY294002 and wortmannin.

In some embodiments, provided herein are pharmaceutical compositions having therapeutically effective amounts of an FTI and a AKT inhibitor, and a pharmaceutically acceptable carrier, diluent or excipient. The AKT inhibitor can be any AKT inhibitor known in the art, including, for example, A-443654, Afuresertib, AMG-511, ARQ-751, AT-13148, AV-203, Capivasertib, CUDC-101, curcumin analog, deguelin, EM12, SK-2636771, GSK-690693, IMB-YH-8, INCB-047775, ipatasertib dihydrochloride, LY-2503029, LY-2780301, miransertib hydrochloride, MK-2206, MK-8156, nanoparticle MK-2206, OB-318, oxysterols, perifosine, PQR-401, PX-316, rigosertib sodium, SR-13668, SZ-685C, TAS-117, Triciribine, Triflorcas, Uprosertib, VEL-015, VLI-27, XL-418. In some embodiments, provided herein are pharmaceutical compositions having therapeutically effective amounts of an FTI and an AKT inhibitor, and a pharmaceutically acceptable carrier, diluent or excipient, wherein the AKT inhibitor is selected from the group consisting of perifosine, SR13668, A-443654, triciribine, GSK690693, and deguelin.

Provided herein are also pharmaceutical compositions having therapeutically effective amounts of an FTI and capecitabine and a pharmaceutically acceptable carrier, diluent or excipient. In some embodiments, the FTI is tipifarnib; lonafarnib (also known as SCH-66336); arglabin; perrilyl alcohol; CP-609,754, BMS 214662; L778123; L744832; L739749; R208176; AZD3409; or FTI-277. Provided herein are also pharmaceutical compositions having therapeutically effective amounts of tipifarnib and capecitabine, and a pharmaceutically acceptable carrier, diluent or excipient.

Provided herein are also pharmaceutical compositions having therapeutically effective amounts of an FTI and an CXCR4 antagonist and a pharmaceutically acceptable carrier, diluent or excipient. In some embodiments, the FTI is tipifarnib; lonafarnib (also known as SCH-66336); arglabin; perrilyl alcohol; CP-609,754, BMS 214662; L778123; L744832; L739749; R208176; AZD3409; or FTI-277. Provided herein are also pharmaceutical compositions having therapeutically effective amounts of tipifarnib and an inhibitor of an CXCR4 antagonist, and a pharmaceutically acceptable carrier, diluent or excipient.

The CXCR4 antagonist can be any CXCR4 antagonist known in the art, including for example AD-114, ALT-1188, AMD-070, AMD-3100, APH-0812, balixafortide, BKT-140, BKT-170, BL-8040, burixafor, CCRS receptor modulator, cefprozil, chloroquine, hydroxychloroquine, CTCE-0012, CX-549, DBPR-215, D-Lys3 GHRP-6, F-50067, filgrastim, GBV-4086, GMI-1359, KRH-1120, KRH-3166, KRH-3955, LY-2510924, LY-2624587, MEDI-3185, N15P peptide, ND-401, NSC-651016, ONO-7161, PF-06747143, plerixafor, POL-5551, PTX-9908, SDF-1 antibody, T-134, TIQ-15 analogs, Ulocuplumab, USL-311, VIR-5103, vMIP, vMIP-II, X4-136, X4P-001, or X4P-002. In some embodiments, provided herein also pharmaceutical compositions having therapeutically effective amounts of an FTI and an CXCR4 antagonist and a pharmaceutically acceptable carrier, diluent or excipient, wherein the CXCR4 is selected from the group consisting of AMD-3100, BL-8040, chloroquine, and plerixafor.

The FTI, either alone or with a second active agent such as an inhibitor of the IGF1R pathway or a CXCR4 antagonist, can be formulated into suitable pharmaceutical preparations such as solutions, suspensions, tablets, dispersible tablets, pills, capsules, powders, sustained release formulations or elixirs, for oral administration or in sterile solutions or suspensions for ophthalmic or parenteral administration, as well as transdermal patch preparation and dry powder inhalers. Typically the FTI is formulated into pharmaceutical compositions using techniques and procedures well known in the art (see, e.g., Ansel Introduction to Pharmaceutical Dosage Forms, Seventh Edition 1999).

In the compositions, effective concentrations of the FTI, either alone or with a second active agent such as an inhibitor of the IGF1R pathway or a CXCR4 antagonist or capecitabine, and pharmaceutically acceptable salts is (are) mixed with a suitable pharmaceutical carrier or vehicle. In certain embodiments, the concentrations of the FTI, either alone or with a second active agent such as an inhibitor of the IGF1R pathway or a CXCR4 antagonist or capecitabine, in the compositions are effective for delivery of an amount, upon administration, that treats, prevents, or ameliorates one or more of the symptoms and/or progression of cancer, including haematological cancers and solid tumors.

The compositions can be formulated for single dosage administration. To formulate a composition, the weight fraction of the FTI, either alone or with a second active agent such as an inhibitor of the IGF1R pathway or a CXCR4 antagonist or capecitabine, is dissolved, suspended, dispersed or otherwise mixed in a selected vehicle at an effective concentration such that the treated condition is relieved or ameliorated. Pharmaceutical carriers or vehicles suitable for administration include any such carriers known to those skilled in the art to be suitable for the particular mode of administration.

In addition, the FTI, either alone or with a second active agent such as an inhibitor of the IGF1R pathway or a CXCR4 antagonist or capecitabine, can be formulated as the sole pharmaceutically active ingredient in the composition or may be combined with other active ingredients. Liposomal suspensions, including tissue-targeted liposomes, such as tumor-targeted liposomes, may also be suitable as pharmaceutically acceptable carriers. These may be prepared according to methods known to those skilled in the art. For example, liposome formulations may be prepared as known in the art. Briefly, liposomes such as multilamellar vesicles (MLV's) may be formed by drying down egg phosphatidyl choline and brain phosphatidyl serine (7:3 molar ratio) on the inside of a flask. A solution in phosphate buffered saline lacking divalent cations (PBS) is added and the flask shaken until the lipid film is dispersed. The resulting vesicles are washed to remove unencapsulated compound, pelleted by centrifugation, and then resuspended in PBS.

The FTI, either alone or with a second active agent such as an inhibitor of the IGF1R pathway or a CXCR4 antagonist or capecitabine, is included in the pharmaceutically acceptable carrier in an amount sufficient to exert a therapeutically useful effect in the absence of undesirable side effects on the patient treated. The therapeutically effective concentration may be determined empirically by testing the compounds in in vitro and in vivo systems described herein and then extrapolated therefrom for dosages for humans.

The concentration of FTI, either alone or with a second active agent such as an inhibitor of the IGF1R pathway or a CXCR4 antagonist or capecitabine, in the pharmaceutical composition will depend on absorption, tissue distribution, inactivation and excretion rates, the physicochemical characteristics, the dosage schedule, and amount administered as well as other factors known to those of skill in the art. For example, the amount that is delivered is sufficient to ameliorate one or more of the symptoms of cancer, including hematopoietic cancers and solid tumors.

In certain embodiments, a therapeutically effective dosage should produce a serum concentration of active ingredient of from about 0.1 ng/ml to about 50-100 μg/ml. In one embodiment, the pharmaceutical compositions provide a dosage of from about 0.001 mg to about 2000 mg of compound per kilogram of body weight per day. Pharmaceutical dosage unit forms are prepared to provide from about 1 mg to about 1000 mg and in certain embodiments, from about 10 to about 500 mg of the essential active ingredient or a combination of essential ingredients per dosage unit form.

The FTI, either alone or with a second active agent such as an inhibitor of the IGF1R pathway or a CXCR4 antagonist or capecitabine, may be administered at once, or may be divided into a number of smaller doses to be administered at intervals of time. It is understood that the precise dosage and duration of treatment is a function of the disease being treated and may be determined empirically using known testing protocols or by extrapolation from in vivo or in vitro test data. It is to be noted that concentrations and dosage values may also vary with the severity of the condition to be alleviated. It is to be further understood that for any particular subject, specific dosage regimens should be adjusted over time according to the individual need and the professional judgment of the person administering or supervising the administration of the compositions, and that the concentration ranges set forth herein are exemplary only and are not intended to limit the scope or practice of the claimed compositions.

Thus, effective concentrations or amounts of one or more of the compounds described herein or pharmaceutically acceptable salts thereof are mixed with a suitable pharmaceutical carrier or vehicle for systemic, topical or local administration to form pharmaceutical compositions. Compounds are included in an amount effective for ameliorating one or more symptoms of, or for treating, retarding progression, or preventing. The concentration of active compound in the composition will depend on absorption, tissue distribution, inactivation, excretion rates of the active compound, the dosage schedule, amount administered, particular formulation as well as other factors known to those of skill in the art.

The compositions are intended to be administered by a suitable route, including but not limited to orally, parenterally, rectally, topically and locally. For oral administration, capsules and tablets can be formulated. The compositions are in liquid, semi-liquid or solid form and are formulated in a manner suitable for each route of administration.

Solutions or suspensions used for parenteral, intradermal, subcutaneous, or topical application can include any of the following components: a sterile diluent, such as water for injection, saline solution, fixed oil, polyethylene glycol, glycerine, propylene glycol, dimethyl acetamide or other synthetic solvent; antimicrobial agents, such as benzyl alcohol and methyl parabens; antioxidants, such as ascorbic acid and sodium bisulFTIe; chelating agents, such as ethylenediaminetetraacetic acid (EDTA); buffers, such as acetates, citrates and phosphates; and agents for the adjustment of tonicity such as sodium chloride or dextrose. Parenteral preparations can be enclosed in ampules, pens, disposable syringes or single or multiple dose vials made of glass, plastic or other suitable material.

In instances in which the FTI, either alone or with a second active agent such as an inhibitor of the IGF1R pathway or a CXCR4 antagonist or capecitabine, exhibits insufficient solubility, methods for solubilizing compounds can be used. Such methods are known to those of skill in this art, and include, but are not limited to, using cosolvents, such as dimethylsulfoxide (DMSO), using surfactants, such as TWEEN®, or dissolution in aqueous sodium bicarbonate.

Upon mixing or addition of the compound(s), the resulting mixture may be a solution, suspension, emulsion or the like. The form of the resulting mixture depends upon a number of factors, including the intended mode of administration and the solubility of the compound in the selected carrier or vehicle. The effective concentration is sufficient for ameliorating the symptoms of the disease, disorder or condition treated and may be empirically determined.

The pharmaceutical compositions are provided for administration to humans and animals in unit dosage forms, such as tablets, capsules, pills, powders, granules, sterile parenteral solutions or suspensions, and oral solutions or suspensions, and oil water emulsions containing suitable quantities of the compounds or pharmaceutically acceptable salts thereof. The pharmaceutically therapeutically active compounds and salts thereof are formulated and administered in unit dosage forms or multiple dosage forms. Unit dose forms as used herein refer to physically discrete units suitable for human and animal subjects and packaged individually as is known in the art. Each unit dose contains a predetermined quantity of the therapeutically active compound sufficient to produce the desired therapeutic effect, in association with the required pharmaceutical carrier, vehicle or diluent. Examples of unit dose forms include ampules and syringes and individually packaged tablets or capsules. Unit dose forms may be administered in fractions or multiples thereof. A multiple dose form is a plurality of identical unit dosage forms packaged in a single container to be administered in segregated unit dose form. Examples of multiple dose forms include vials, bottles of tablets or capsules or bottles of pints or gallons. Hence, multiple dose form is a multiple of unit doses which are not segregated in packaging.

Sustained-release preparations can also be prepared. Suitable examples of sustained-release preparations include semipermeable matrices of solid hydrophobic polymers containing the compound provided herein, which matrices are in the form of shaped articles, e.g., films, or microcapsule. Examples of sustained-release matrices include iontophoresis patches, polyesters, hydrogels (for example, poly(2-hydroxyethyl-methacrylate), or poly(vinylalcohol)), polylactides, copolymers of L-glutamic acid and ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), and poly-D-(−)-3-hydroxybutyric acid. While polymers such as ethylene-vinyl acetate and lactic acid-glycolic acid enable release of molecules for over 100 days, certain hydrogels release proteins for shorter time periods. When encapsulated compound remain in the body for a long time, they may denature or aggregate as a result of exposure to moisture at 37° C., resulting in a loss of biological activity and possible changes in their structure. Rational strategies can be devised for stabilization depending on the mechanism of action involved. For example, if the aggregation mechanism is discovered to be intermolecular S--S bond formation through thio-disulfide interchange, stabilization may be achieved by modifying sulfhydryl residues, lyophilizing from acidic solutions, controlling moisture content, using appropriate additives, and developing specific polymer matrix compositions.

Dosage forms or compositions containing active ingredient in the range of 0.005% to 100% with the balance made up from nontoxic carrier may be prepared. For oral administration, a pharmaceutically acceptable nontoxic composition is formed by the incorporation of any of the normally employed excipients, such as, for example pharmaceutical grades of mannitol, lactose, starch, magnesium stearate, talcum, cellulose derivatives, sodium crosscarmellose, glucose, sucrose, magnesium carbonate or sodium saccharin. Such compositions include solutions, suspensions, tablets, capsules, powders and sustained release formulations, such as, but not limited to, implants and microencapsulated delivery systems, and biodegradable, biocompatible polymers, such as collagen, ethylene vinyl acetate, polyanhydrides, polyglycolic acid, polyorthoesters, polylactic acid and others. Methods for preparation of these compositions are known to those skilled in the art. The contemplated compositions may contain about 0.001% 100% active ingredient, in certain embodiments, about 0.1-85% or about 75-95%.

The FTI, either alone or with a second active agent such as an inhibitor of the IGF1R pathway or a CXCR4 antagonist or capecitabine, or pharmaceutically acceptable salts can be prepared with carriers that protect the compound against rapid elimination from the body, such as time release formulations or coatings.

The compositions can include other active compounds to obtain desired combinations of properties. The compounds provided herein, or pharmaceutically acceptable salts thereof as described herein, can also be administered together with another pharmacological agent known in the general art to be of value in treating one or more of the diseases or medical conditions referred to hereinabove, such as diseases related to oxidative stress.

Lactose-free compositions provided herein can contain excipients that are well known in the art and are listed, for example, in the U.S. Pharmocopia (USP) SP (XXI)/NF (XVI). In general, lactose-free compositions contain an active ingredient, a binder/filler, and a lubricant in pharmaceutically compatible and pharmaceutically acceptable amounts. Exemplary lactose-free dosage forms contain an active ingredient, microcrystalline cellulose, pre-gelatinized starch and magnesium stearate.

Further encompassed are anhydrous pharmaceutical compositions and dosage forms containing a compound provided herein. For example, the addition of water (e.g., 5%) is widely accepted in the pharmaceutical arts as a means of simulating long-term storage in order to determine characteristics such as shelf-life or the stability of formulations over time. See, e.g., Jens T. Carstensen, Drug Stability: Principles & Practice, 2d. Ed., Marcel Dekker, NY, NY, 1995, pp. 379-80. In effect, water and heat accelerate the decomposition of some compounds. Thus, the effect of water on a formulation can be of great significance since moisture and/or humidity are commonly encountered during manufacture, handling, packaging, storage, shipment and use of formulations.

Anhydrous pharmaceutical compositions and dosage forms provided herein can be prepared using anhydrous or low moisture containing ingredients and low moisture or low humidity conditions. Pharmaceutical compositions and dosage forms that comprise lactose and at least one active ingredient that comprises a primary or secondary amine are anhydrous if substantial contact with moisture and/or humidity during manufacturing, packaging, and/or storage is expected.

An anhydrous pharmaceutical composition should be prepared and stored such that its anhydrous nature is maintained. Accordingly, anhydrous compositions are packaged using materials known to prevent exposure to water such that they can be included in suitable formulary kits. Examples of suitable packaging include, but are not limited to, hermetically sealed foils, plastics, unit dose containers (e.g., vials), blister packs and strip packs.

Oral pharmaceutical dosage forms are either solid, gel or liquid. The solid dosage forms are tablets, capsules, granules, and bulk powders. Types of oral tablets include compressed, chewable lozenges and tablets which may be enteric coated, sugar coated or film coated. Capsules may be hard or soft gelatin capsules, while granules and powders may be provided in noneffervescent or effervescent form with the combination of other ingredients known to those skilled in the art.

In certain embodiments, the formulations are solid dosage forms, such as capsules or tablets. The tablets, pills, capsules, troches and the like can contain any of the following ingredients, or compounds of a similar nature: a binder; a diluent; a disintegrating agent; a lubricant; a glidant; a sweetening agent; and a flavoring agent.

Examples of binders include microcrystalline cellulose, gum tragacanth, glucose solution, acacia mucilage, gelatin solution, sucrose and starch paste. Lubricants include talc, starch, magnesium or calcium stearate, lycopodium and stearic acid. Diluents include, for example, lactose, sucrose, starch, kaolin, salt, mannitol and dicalcium phosphate. Glidants include, but are not limited to, colloidal silicon dioxide. Disintegrating agents include crosscarmellose sodium, sodium starch glycolate, alginic acid, corn starch, potato starch, bentonite, methylcellulose, agar and carboxymethylcellulose. Coloring agents include, for example, any of the approved certified water soluble FD and C dyes, mixtures thereof; and water insoluble FD and C dyes suspended on alumina hydrate. Sweetening agents include sucrose, lactose, mannitol and artificial sweetening agents such as saccharin, and any number of spray dried flavors. Flavoring agents include natural flavors extracted from plants such as fruits and synthetic blends of compounds which produce a pleasant sensation, such as, but not limited to peppermint and methyl salicylate. Wetting agents include propylene glycol monostearate, sorbitan monooleate, diethylene glycol monolaurate and polyoxyethylene laural ether. Emetic coatings include fatty acids, fats, waxes, shellac, ammoniated shellac and cellulose acetate phthalates. Film coatings include hydroxyethylcellulose, sodium carboxymethylcellulose, polyethylene glycol 4000 and cellulose acetate phthalate.

When the dosage unit form is a capsule, it can contain, in addition to material of the above type, a liquid carrier such as a fatty oil. In addition, dosage unit forms can contain various other materials which modify the physical form of the dosage unit, for example, coatings of sugar and other enteric agents. The compounds can also be administered as a component of an elixir, suspension, syrup, wafer, sprinkle, chewing gum or the like. A syrup may contain, in addition to the active compounds, sucrose as a sweetening agent and certain preservatives, dyes and colorings and flavors.

Pharmaceutically acceptable carriers included in tablets are binders, lubricants, diluents, disintegrating agents, coloring agents, flavoring agents, and wetting agents. Enteric coated tablets, because of the enteric coating, resist the action of stomach acid and dissolve or disintegrate in the neutral or alkaline intestines. Sugar coated tablets are compressed tablets to which different layers of pharmaceutically acceptable substances are applied. Film coated tablets are compressed tablets which have been coated with a polymer or other suitable coating. Multiple compressed tablets are compressed tablets made by more than one compression cycle utilizing the pharmaceutically acceptable substances previously mentioned. Coloring agents may also be used in the above dosage forms. Flavoring and sweetening agents are used in compressed tablets, sugar coated, multiple compressed and chewable tablets. Flavoring and sweetening agents are especially useful in the formation of chewable tablets and lozenges.

Liquid oral dosage forms include aqueous solutions, emulsions, suspensions, solutions and/or suspensions reconstituted from non effervescent granules and effervescent preparations reconstituted from effervescent granules. Aqueous solutions include, for example, elixirs and syrups. Emulsions are either oil in-water or water in oil.

Elixirs are clear, sweetened, hydroalcoholic preparations. Pharmaceutically acceptable carriers used in elixirs include solvents. Syrups are concentrated aqueous solutions of a sugar, for example, sucrose, and may contain a preservative. An emulsion is a two phase system in which one liquid is dispersed in the form of small globules throughout another liquid. Pharmaceutically acceptable carriers used in emulsions are non aqueous liquids, emulsifying agents and preservatives. Suspensions use pharmaceutically acceptable suspending agents and preservatives. Pharmaceutically acceptable substances used in non effervescent granules, to be reconstituted into a liquid oral dosage form, include diluents, sweeteners and wetting agents. Pharmaceutically acceptable substances used in effervescent granules, to be reconstituted into a liquid oral dosage form, include organic acids and a source of carbon dioxide. Coloring and flavoring agents are used in all of the above dosage forms.

Solvents include glycerin, sorbitol, ethyl alcohol and syrup. Examples of preservatives include glycerin, methyl and propylparaben, benzoic add, sodium benzoate and alcohol. Examples of non aqueous liquids utilized in emulsions include mineral oil and cottonseed oil. Examples of emulsifying agents include gelatin, acacia, tragacanth, bentonite, and surfactants such as polyoxyethylene sorbitan monooleate. Suspending agents include sodium carboxymethylcellulose, pectin, tragacanth, Veegum and acacia. Diluents include lactose and sucrose. Sweetening agents include sucrose, syrups, glycerin and artificial sweetening agents such as saccharin. Wetting agents include propylene glycol monostearate, sorbitan monooleate, diethylene glycol monolaurate and polyoxyethylene lauryl ether. Organic adds include citric and tartaric acid. Sources of carbon dioxide include sodium bicarbonate and sodium carbonate. Coloring agents include any of the approved certified water soluble FD and C dyes, and mixtures thereof. Flavoring agents include natural flavors extracted from plants such fruits, and synthetic blends of compounds which produce a pleasant taste sensation.

For a solid dosage form, the solution or suspension, in for example propylene carbonate, vegetable oils or triglycerides, is encapsulated in a gelatin capsule. Such solutions, and the preparation and encapsulation thereof, are disclosed in U.S. Pat. Nos. 4,328,245; 4,409,239; and 4,410,545. For a liquid dosage form, the solution, e.g., for example, in a polyethylene glycol, may be diluted with a sufficient quantity of a pharmaceutically acceptable liquid carrier, e.g., water, to be easily measured for administration.

Alternatively, liquid or semi solid oral formulations may be prepared by dissolving or dispersing the active compound or salt in vegetable oils, glycols, triglycerides, propylene glycol esters (e.g., propylene carbonate) and other such carriers, and encapsulating these solutions or suspensions in hard or soft gelatin capsule shells. Other useful formulations include, but are not limited to, those containing a compound provided herein, a dialkylated mono- or poly-alkylene glycol, including, but not limited to, 1,2-dimethoxymethane, diglyme, triglyme, tetraglyme, polyethylene glycol-350-dimethyl ether, polyethylene glycol-550-dimethyl ether, polyethylene glycol-750-dimethyl ether wherein 350, 550 and 750 refer to the approximate average molecular weight of the polyethylene glycol, and one or more antioxidants, such as butylated hydroxytoluene (BHT), butylated hydroxyanisole (BHA), propyl gallate, vitamin E, hydroquinone, hydroxycoumarins, ethanolamine, lecithin, cephalin, ascorbic acid, malic acid, sorbitol, phosphoric acid, thiodipropionic acid and its esters, and dithiocarbamates.

Other formulations include, but are not limited to, aqueous alcoholic solutions including a pharmaceutically acceptable acetal. Alcohols used in these formulations are any pharmaceutically acceptable water-miscible solvents having one or more hydroxyl groups, including, but not limited to, propylene glycol and ethanol. Acetals include, but are not limited to, di(lower alkyl) acetals of lower alkyl aldehydes such as acetaldehyde diethyl acetal.

In all embodiments, tablets and capsules formulations may be coated as known by those of skill in the art in order to modify or sustain dissolution of the active ingredient. Thus, for example, they may be coated with a conventional enterically digestible coating, such as phenylsalicylate, waxes and cellulose acetate phthalate.

Parenteral administration, generally characterized by injection, either subcutaneously, intramuscularly or intravenously is also provided herein. Injectables can be prepared in conventional forms, either as liquid solutions or suspensions, solid forms suitable for solution or suspension in liquid prior to injection, or as emulsions. Suitable excipients are, for example, water, saline, dextrose, glycerol or ethanol. In addition, if desired, the pharmaceutical compositions to be administered may also contain minor amounts of non toxic auxiliary substances such as wetting or emulsifying agents, pH buffering agents, stabilizers, solubility enhancers, and other such agents, such as for example, sodium acetate, sorbitan monolaurate, triethanolamine oleate and cyclodextrins. Implantation of a slow release or sustained release system, such that a constant level of dosage is maintained is also contemplated herein. Briefly, a compound provided herein is dispersed in a solid inner matrix, e.g., polymethylmethacrylate, polybutylmethacrylate, plasticized or unplasticized polyvinylchloride, plasticized nylon, plasticized polyethyleneterephthalate, natural rubber, polyisoprene, polyisobutylene, polybutadiene, polyethylene, ethylene-vinylacetate copolymers, silicone rubbers, polydimethylsiloxanes, silicone carbonate copolymers, hydrophilic polymers such as hydrogels of esters of acrylic and methacrylic acid, collagen, cross-linked polyvinylalcohol and cross-linked partially hydrolyzed polyvinyl acetate, that is surrounded by an outer polymeric membrane, e.g., polyethylene, polypropylene, ethylene/propylene copolymers, ethylene/ethyl acrylate copolymers, ethylene/vinylacetate copolymers, silicone rubbers, polydimethyl siloxanes, neoprene rubber, chlorinated polyethylene, polyvinylchloride, vinylchloride copolymers with vinyl acetate, vinylidene chloride, ethylene and propylene, ionomer polyethylene terephthalate, butyl rubber epichlorohydrin rubbers, ethylene/vinyl alcohol copolymer, ethylene/vinyl acetate/vinyl alcohol terpolymer, and ethylene/vinyloxyethanol copolymer, that is insoluble in body fluids. The compound diffuses through the outer polymeric membrane in a release rate controlling step. The percentage of active compound contained in such parenteral compositions is highly dependent on the specific nature thereof, as well as the activity of the compound and the needs of the subject.

Parenteral administration of the compositions includes intravenous, subcutaneous and intramuscular administrations. Preparations for parenteral administration include sterile solutions ready for injection, sterile dry soluble products, such as lyophilized powders, ready to be combined with a solvent just prior to use, including hypodermic tablets, sterile suspensions ready for injection, sterile dry insoluble products ready to be combined with a vehicle just prior to use and sterile emulsions. The solutions may be either aqueous or nonaqueous.

If administered intravenously, suitable carriers include physiological saline or phosphate buffered saline (PBS), and solutions containing thickening and solubilizing agents, such as glucose, polyethylene glycol, and polypropylene glycol and mixtures thereof.

Pharmaceutically acceptable carriers used in parenteral preparations include aqueous vehicles, nonaqueous vehicles, antimicrobial agents, isotonic agents, buffers, antioxidants, local anesthetics, suspending and dispersing agents, emulsifying agents, sequestering or chelating agents and other pharmaceutically acceptable substances.

Examples of aqueous vehicles include Sodium Chloride Injection, Ringers Injection, Isotonic Dextrose Injection, Sterile Water Injection, Dextrose and Lactated Ringers Injection. Nonaqueous parenteral vehicles include fixed oils of vegetable origin, cottonseed oil, corn oil, sesame oil and peanut oil. Antimicrobial agents in bacteriostatic or fungistatic concentrations must be added to parenteral preparations packaged in multiple dose containers which include phenols or cresols, mercurials, benzyl alcohol, chlorobutanol, methyl and propyl p hydroxybenzoic acid esters, thimerosal, benzalkonium chloride and benzethonium chloride. Isotonic agents include sodium chloride and dextrose. Buffers include phosphate and citrate. Antioxidants include sodium bisulfate. Local anesthetics include procaine hydrochloride. Suspending and dispersing agents include sodium carboxymethylcelluose, hydroxypropyl methylcellulose and polyvinylpyrrolidone. Emulsifying agents include Polysorbate 80 (TWEEN® 80). A sequestering or chelating agent of metal ions include EDTA. Pharmaceutical carriers also include ethyl alcohol, polyethylene glycol and propylene glycol for water miscible vehicles and sodium hydroxide, hydrochloric acid, citric acid or lactic acid for pH adjustment.

The concentration of the FTI is adjusted so that an injection provides an effective amount to produce the desired pharmacological effect. The exact dose depends on the age, weight and condition of the patient or animal as is known in the art. The unit dose parenteral preparations are packaged in an ampule, a vial or a syringe with a needle. All preparations for parenteral administration must be sterile, as is known and practiced in the art.

Illustratively, intravenous or intraarterial infusion of a sterile aqueous solution containing an FTI is an effective mode of administration. Another embodiment is a sterile aqueous or oily solution or suspension containing an active material injected as necessary to produce the desired pharmacological effect.

Injectables are designed for local and systemic administration. Typically a therapeutically effective dosage is formulated to contain a concentration of at least about 0.1% w/w up to about 90% w/w or more, such as more than 1% w/w of the active compound to the treated tissue(s). The active ingredient may be administered at once, or may be divided into a number of smaller doses to be administered at intervals of time. It is understood that the precise dosage and duration of treatment is a function of the tissue being treated and may be determined empirically using known testing protocols or by extrapolation from in vivo or in vitro test data. It is to be noted that concentrations and dosage values may also vary with the age of the individual treated. It is to be further understood that for any particular subject, specific dosage regimens should be adjusted over time according to the individual need and the professional judgment of the person administering or supervising the administration of the formulations, and that the concentration ranges set forth herein are exemplary only and are not intended to limit the scope or practice of the claimed formulations.

The FTI, either alone or with a second active agent such as an inhibitor of the IGF1R pathway or a CXCR4 antagonist or capecitabine, can be suspended in micronized or other suitable form or may be derivatized to produce a more soluble active product or to produce a prodrug. The form of the resulting mixture depends upon a number of factors, including the intended mode of administration and the solubility of the compound in the selected carrier or vehicle. The effective concentration is sufficient for ameliorating the symptoms of the condition and may be empirically determined.

Of interest herein are also lyophilized powders, which can be reconstituted for administration as solutions, emulsions and other mixtures. They can also be reconstituted and formulated as solids or gels.

The sterile, lyophilized powder is prepared by dissolving an FTI, either alone or with a second active agent such as an inhibitor of the IGF1R pathway or a CXCR4 antagonist or capecitabine, provided herein, or a pharmaceutically acceptable salt thereof, in a suitable solvent. The solvent may contain an excipient which improves the stability or other pharmacological component of the powder or reconstituted solution, prepared from the powder. Excipients that may be used include, but are not limited to, dextrose, sorbital, fructose, corn syrup, xylitol, glycerin, glucose, sucrose or other suitable agent. The solvent may also contain a buffer, such as citrate, sodium or potassium phosphate or other such buffer known to those of skill in the art at, in one embodiment, about neutral pH. Subsequent sterile filtration of the solution followed by lyophilization under standard conditions known to those of skill in the art provides the desired formulation. Generally, the resulting solution will be apportioned into vials for lyophilization. Each vial will contain a single dosage (including but not limited to 10-1000 mg or 100-500 mg) or multiple dosages of the compound. The lyophilized powder can be stored under appropriate conditions, such as at about 4° C. to room temperature.

Reconstitution of this lyophilized powder with water for injection provides a formulation for use in parenteral administration. For reconstitution, about 1-50 mg, about 5-35 mg, or about 9-30 mg of lyophilized powder, is added per mL of sterile water or other suitable carrier. The precise amount depends upon the selected compound. Such amount can be empirically determined.

Topical mixtures are prepared as described for the local and systemic administration. The resulting mixture may be a solution, suspension, emulsion or the like and are formulated as creams, gels, ointments, emulsions, solutions, elixirs, lotions, suspensions, tinctures, pastes, foams, aerosols, irrigations, sprays, suppositories, bandages, dermal patches or any other formulations suitable for topical administration.

The FTI or pharmaceutical composition having an FTI can be formulated as aerosols for topical application, such as by inhalation (see, e.g., U.S. Pat. Nos. 4,044,126, 4,414,209, and 4,364,923, which describe aerosols for delivery of a steroid useful for treatment of inflammatory diseases, particularly asthma). These formulations for administration to the respiratory tract can be in the form of an aerosol or solution for a nebulizer, or as a microfine powder for insufflation, alone or in combination with an inert carrier such as lactose. In such a case, the particles of the formulation will have diameters of less than 50 microns or less than 10 microns.

The FTI, either alone or with a second active agent such as an inhibitor of the IGF1R pathway or a CXCR4 antagonist or capecitabine, or pharmaceutical composition thereof, can be formulated for local or topical application, such as for topical application to the skin and mucous membranes, such as in the eye, in the form of gels, creams, and lotions and for application to the eye or for intracisternal or intraspinal application. Topical administration is contemplated for transdermal delivery and also for administration to the eyes or mucosa, or for inhalation therapies. Nasal solutions of the active compound alone or in combination with other pharmaceutically acceptable excipients can also be administered. These solutions, particularly those intended for ophthalmic use, may be formulated as 0.01%-10% isotonic solutions, pH about 5-7, with appropriate salts.

Other routes of administration, such as transdermal patches, and rectal administration are also contemplated herein. For example, pharmaceutical dosage forms for rectal administration are rectal suppositories, capsules and tablets for systemic effect. Rectal suppositories are used herein mean solid bodies for insertion into the rectum which melt or soften at body temperature releasing one or more pharmacologically or therapeutically active ingredients. Pharmaceutically acceptable substances utilized in rectal suppositories are bases or vehicles and agents to raise the melting point. Examples of bases include cocoa butter (theobroma oil), glycerin gelatin, carbowax (polyoxyethylene glycol) and appropriate mixtures of mono, di and triglycerides of fatty acids. Combinations of the various bases may be used. Agents to raise the melting point of suppositories include spermaceti and wax. Rectal suppositories may be prepared either by the compressed method or by molding. An exemplary weight of a rectal suppository is about 2 to 3 grams. Tablets and capsules for rectal administration are manufactured using the same pharmaceutically acceptable substance and by the same methods as for formulations for oral administration.

The FTI, either alone or with a second active agent such as an inhibitor of the IGF1R pathway or a CXCR4 antagonist or capecitabine, or pharmaceutical composition thereof, provided herein can be administered by controlled release means or by delivery devices that are well known to those of ordinary skill in the art. Examples include, but are not limited to, those described in U.S. Pat. Nos. 3,845,770; 3,916,899; 3,536,809; 3,598,123; and 4,008,719, 5,674,533, 5,059,595, 5,591,767, 5,120,548, 5,073,543, 5,639,476, 5,354,556, 5,639,480, 5,733,566, 5,739,108, 5,891,474, 5,922,356, 5,972,891, 5,980,945, 5,993,855, 6,045,830, 6,087,324, 6,113,943, 6,197,350, 6,248,363, 6,264,970, 6,267,981, 6,376,461,6,419,961, 6,589,548, 6,613,358, 6,699,500 and 6,740,634, each of which is incorporated herein by reference. Such dosage forms can be used to provide slow or controlled-release using, for example, hydropropylmethyl cellulose, other polymer matrices, gels, permeable membranes, osmotic systems, multilayer coatings, microparticles, liposomes, microspheres, or a combination thereof to provide the desired release profile in varying proportions. Suitable controlled-release formulations known to those of ordinary skill in the art, including those described herein, can be readily selected for use with the active ingredients provided herein.

All controlled-release pharmaceutical products have a common goal of improving drug therapy over that achieved by their non-controlled counterparts. In one embodiment, the use of an optimally designed controlled-release preparation in medical treatment is characterized by a minimum of drug substance being employed to cure or control the condition in a minimum amount of time. In certain embodiments, advantages of controlled-release formulations include extended activity of the drug, reduced dosage frequency, and increased patient compliance. In addition, controlled-release formulations can be used to affect the time of onset of action or other characteristics, such as blood levels of the drug, and can thus affect the occurrence of side (e.g., adverse) effects.

Most controlled-release formulations are designed to initially release an amount of drug (active ingredient) that promptly produces the desired therapeutic effect, and gradually and continually release of other amounts of drug to maintain this level of therapeutic effect over an extended period of time. In order to maintain this constant level of drug in the body, the drug must be released from the dosage form at a rate that will replace the amount of drug being metabolized and excreted from the body. Controlled-release of an active ingredient can be stimulated by various conditions including, but not limited to, pH, temperature, enzymes, water, or other physiological conditions or compounds.

In certain embodiments, the FTI, either alone or with a second active agent such as an inhibitor of the IGF1R pathway or a CXCR4 antagonist or capecitabine, can be administered using intravenous infusion, an implantable osmotic pump, a transdermal patch, liposomes, or other modes of administration. In one embodiment, a pump may be used (see, Sefton, CRC Crit. Ref. Biomed. Eng. 14:201 (1987); Buchwald et al., Surgery 88:507 (1980); Saudek et al., N. Engl. J. Med. 321:574 (1989). In another embodiment, polymeric materials can be used. In yet another embodiment, a controlled release system can be placed in proximity of the therapeutic target, i.e., thus requiring only a fraction of the systemic dose (see, e.g., Goodson, Medical Applications of Controlled Release, vol. 2, pp. 115-138 (1984).

In some embodiments, a controlled release device is introduced into a subject in proximity of the site of inappropriate immune activation or a tumor. Other controlled release systems are discussed in the review by Langer (Science 249:1527-1533 (1990). The F can be dispersed in a solid inner matrix, e.g., polymethylmethacrylate, polybutylmethacrylate, plasticized or unplasticized polyvinylchloride, plasticized nylon, plasticized polyethyleneterephthalate, natural rubber, polyisoprene, polyisobutylene, polybutadiene, polyethylene, ethylene-vinylacetate copolymers, silicone rubbers, polydimethylsiloxanes, silicone carbonate copolymers, hydrophilic polymers such as hydrogels of esters of acrylic and methacrylic acid, collagen, cross-linked polyvinylalcohol and cross-linked partially hydrolyzed polyvinyl acetate, that is surrounded by an outer polymeric membrane, e.g., polyethylene, polypropylene, ethylene/propylene copolymers, ethylene/ethyl acrylate copolymers, ethylene/vinylacetate copolymers, silicone rubbers, polydimethyl siloxanes, neoprene rubber, chlorinated polyethylene, polyvinylchloride, vinylchloride copolymers with vinyl acetate, vinylidene chloride, ethylene and propylene, ionomer polyethylene terephthalate, butyl rubber epichlorohydrin rubbers, ethylene/vinyl alcohol copolymer, ethylene/vinyl acetate/vinyl alcohol terpolymer, and ethylene/vinyloxyethanol copolymer, that is insoluble in body fluids. The active ingredient then diffuses through the outer polymeric membrane in a release rate controlling step. The percentage of active ingredient contained in such parenteral compositions is highly dependent on the specific nature thereof, as well as the needs of the subject.

The FTI, either alone or with a second active agent such as an inhibitor of the IGF1R pathway or a CXCR4 antagonist or capecitabine, or pharmaceutical composition thereof can be packaged as articles of manufacture containing packaging material, a compound or pharmaceutically acceptable salt thereof provided herein, which is used for treatment, prevention or amelioration of one or more symptoms or progression of cancer, including haematological cancers and solid tumors, and a label that indicates that the compound or pharmaceutically acceptable salt thereof is used for treatment, prevention or amelioration of one or more symptoms or progression of cancer, including haematological cancers and solid tumors.

The articles of manufacture provided herein contain packaging materials. Packaging materials for use in packaging pharmaceutical products are well known to those of skill in the art. See, e.g., U.S. Pat. Nos. 5,323,907, 5,052,558 and 5,033,252. Examples of pharmaceutical packaging materials include, but are not limited to, blister packs, bottles, tubes, inhalers, pumps, bags, vials, containers, syringes, pens, bottles, and any packaging material suitable for a selected formulation and intended mode of administration and treatment. A wide array of formulations of the compounds and compositions provided herein are contemplated.

### 4. DOSAGES OF FTI

In some embodiments, a therapeutically effective amount of the pharmaceutical composition having an FTI is administered orally or parenterally.

In some embodiments, the FTI is administered at a daily dose of from 0.05 up to 1800 mg/kg. In some embodiments, the FTI is administered at a daily dose of from 0.05 up to 1500 mg/kg. In some embodiments, the FTI is administered at a daily dose of from 0.05 up to 500 mg/kg. In some embodiments, the FTI is administered in an amount of 0.05 mg/kg daily, 0.1 mg/kg daily, 0.2 mg/kg daily, 0.5 mg/kg daily, 1 mg/kg daily, 2 mg/kg daily, 5 mg/kg daily, 10 mg/kg daily, 20 mg/kg daily, 50 mg/kg daily, 100 mg/kg daily, 200 mg/kg daily, 300 mg/kg daily, 400 mg/kg daily, 500 mg/kg daily, 600 mg/kg daily, 700 mg/kg daily, 800 mg/kg daily, 900 mg/kg daily, 1000 mg/kg daily, 1100 mg/kg daily, 1200 mg/kg daily, 1300 mg/kg daily, 1400 mg/kg daily, or 1500 mg/kg daily. In some embodiments, the FTI is administered at 1 mg/kg daily. In some embodiments, the FTI is administered at 2 mg/kg daily. In some embodiments, the FTI is administered at 5 mg/kg daily. In some embodiments, the FTI is administered at 10 mg/kg daily. In some embodiments, the FTI is administered at 20 mg/kg daily. In some embodiments, the FTI is administered at 50 mg/kg daily. In some embodiments, the FTI is administered at 100 mg/kg daily. In some embodiments, the FTI is administered at 200 mg/kg daily. In some embodiments, the FTI is administered at 500 mg/kg daily. The FTI can be administered either as a single dose or subdivided into more than one dose. In some embodiments, the FTI is tipifarnib.

In some embodiments, the FTI is administered at a dose of 50-2400 mg daily. In some embodiments, the FTI is administered at a dose of 100-1800 mg daily. In some embodiments, the FTI is administered at a dose of 100-1200 mg daily. In some embodiments, the FTI is administered at a dose of 50 mg, 100 mg, 200 mg, 300 mg, 400 mg, 500 mg, 600 mg, 700 mg, 800 mg, 900 mg, 1000 mg, 1100 mg, 1200 mg, 1300 mg, 1400 mg, 1500 mg, 1600 mg, 1700 mg, 1800 mg, 1900 mg, 2000 mg, 2100 mg, 2200 mg, 1200 mg, or 2400 mg daily. In some embodiments, the FTI is administered at a dose of 200 mg daily. The FTI can be administered at a dose of 300 mg daily. The FTI can be administered at a dose of 400 mg daily. The FTI can be administered at a dose of 500 mg daily. The FTI can be administered at a dose of 600 mg daily. The FTI can be administered at a dose of 700 mg daily. The FTI can be administered at a dose of 800 mg daily. The FTI can be administered at a dose of 900 mg daily. The FTI can be administered at a dose of 1000 mg daily. The FTI can be administered at a dose of 1100 mg daily. The FTI can be administered at a dose of 1200 mg daily. The FTI can be administered at a dose of 1300 mg daily. The FTI can be administered at a dose of 1400 mg daily. The FTI can be administered at a dose of 1500 mg daily. The FTI can be administered at a dose of 1600 mg daily. The FTI can be administered at a dose of 1700 mg daily. The FTI can be administered at a dose of 1800 mg daily. The FTI can be administered at a dose of 1900 mg daily. The FTI can be administered at a dose of 2000 mg daily. The FTI can be administered at a dose of 2100 mg daily. The FTI can be administered at a dose of 2200 mg daily. The FTI can be administered at a dose of 2300 mg daily. The FTI can be administered at a dose of 2400 mg daily. The FTI can be administered either as a single dose or subdivided into more than one dose. In some embodiments, the FTI is tipifarnib.

In some embodiments, an FTI is administered at a dose of 100, 200, 225, 250, 275, 300, 325, 350, 375, 400, 425, 450, 475, 500, 525, 550, 575, 600, 625, 650, 675, 700, 725, 750, 775, 800, 825, 850, 875, 900, 925, 950, 975, 1000, 1025, 1050, 1075, 1100, 1125, 1150, 1175, or 1200 mg twice a day (b.i.d). In some embodiments, the FTI is administered at a dose of 100-1400 mg b.i.d. In some embodiments, the FTI is administered at a dose of 100-1200 mg b.i.d. In some embodiments, the FTI is administered at a dose of 300-1200 mg b.i.d. In some embodiments, the FTI is administered at a dose of 300-900 mg b.i.d. In some embodiments, the FTI is administered at a dose of 300 mg b.i.d. In some embodiments, the FTI is administered at a dose of 400 mg b.i.d. In some embodiments, the FTI is administered at a dose of 500 mg b.i.d. In some embodiments, the FTI is administered at a dose of 600 mg b.i.d. In some embodiments, the FTI is administered at a dose of 700 mg b.i.d. In some embodiments, the FTI is administered at a dose of 800 mg b.i.d. In some embodiments, the FTI is administered at a dose of 900 mg b.i.d. In some embodiments, the FTI is administered at a dose of 1000 mg b.i.d. In some embodiments, the FTI is administered at a dose of 1100 mg b.i.d. In some embodiments, the FTI is administered at a dose of 1200 mg b.i.d. In some embodiments, the FTI for use in the compositions and methods provided herein is tipifarnib.

As a person of ordinary skill in the art would understand, the dosage varies depending on the dosage form employed, condition and sensitivity of the patient, the route of administration, and other factors. The exact dosage will be determined by the practitioner, in light of factors related to the subject that requires treatment. Dosage and administration are adjusted to provide sufficient levels of the active ingredient or to maintain the desired effect. Factors which can be taken into account include the severity of the disease state, general health of the subject, age, weight, and gender of the subject, diet, time and frequency of administration, drug combination(s), reaction sensitivities, and tolerance/response to therapy. During a treatment cycle, the daily dose could be varied. In some embodiments, a starting dosage can be titrated down within a treatment cycle. In some embodiments, a starting dosage can be titrated up within a treatment cycle. The final dosage can depend on the occurrence of dose limiting toxicity and other factors. In some embodiments, the FTI is administered at a starting dose of 300 mg daily and escalated to a maximum dose of 400 mg, 500 mg, 600 mg, 700 mg, 800 mg, 900 mg, 1000 mg, 1100 mg, or 1200 mg daily. In some embodiments, the FTI is administered at a starting dose of 400 mg daily and escalated to a maximum dose of 500 mg, 600 mg, 700 mg, 800 mg, 900 mg, 1000 mg, 1100 mg, or 1200 mg daily. In some embodiments, the FTI is administered at a starting dose of 500 mg daily and escalated to a maximum dose of 600 mg, 700 mg, 800 mg, 900 mg, 1000 mg, 1100 mg, or 1200 mg daily. In some embodiments, the FTI is administered at a starting dose of 600 mg daily and escalated to a maximum dose of 700 mg, 800 mg, 900 mg, 1000 mg, 1100 mg, or 1200 mg daily. In some embodiments, the FTI is administered at a starting dose of 700 mg daily and escalated to a maximum dose of 800 mg, 900 mg, 1000 mg, 1100 mg, or 1200 mg daily. In some embodiments, the FTI is administered at a starting dose of 800 mg daily and escalated to a maximum dose of 900 mg, 1000 mg, 1100 mg, or 1200 mg daily. In some embodiments, the FTI is administered at a starting dose of 900 mg daily and escalated to a maximum dose of 1000 mg, 1100 mg, or 1200 mg daily. The dose escalation can be done at once, or step wise. For example, a starting dose at 600 mg daily can be escalated to a final dose of 1000 mg daily by increasing by 100 mg per day over the course of 4 days, or by increasing by 200 mg per day over the course of 2 days, or by increasing by 400 mg at once. In some embodiments, the FTI is tipifarnib.

In some embodiments, the FTI is administered at a relatively high starting dose and titrated down to a lower dose depending on the patient response and other factors. In some embodiments, the FTI is administered at a starting dose of 1200 mg daily and reduced to a final dose of 1100 mg, 1000 mg, 900 mg, 800 mg, 700 mg, 600 mg, 500 mg, 400 mg, or 300 mg daily. In some embodiments, the FTI is administered at a starting dose of 1100 mg daily and reduced to a final dose of 1000 mg, 900 mg, 800 mg, 700 mg, 600 mg, 500 mg, 400 mg, or 300 mg daily. In some embodiments, the FTI is administered at a starting dose of 1000 mg daily and reduced to a final dose of 900 mg, 800 mg, 700 mg, 600 mg, 500 mg, 400 mg, or 300 mg daily. In some embodiments, the FTI is administered at a starting dose of 900 mg daily and reduced to a final dose of 800 mg, 700 mg, 600 mg, 500 mg, 400 mg, or 300 mg daily. In some embodiments, the FTI is administered at a starting dose of 800 mg daily and reduced to a final dose of 700 mg, 600 mg, 500 mg, 400 mg, or 300 mg daily. In some embodiments, the FTI is administered at a starting dose of 600 mg daily and reduced to a final dose of 500 mg, 400 mg, or 300 mg daily. The dose reduction can be done at once, or step wise. In some embodiments, the FTI is tipifarnib. For example, a starting dose at 900 mg daily can be reduced to a final dose of 600 mg daily by decreasing by 100 mg per day over the course of 3 days, or by decreasing by 300 mg at once. In some embodiments, the FTI is tipifarnib.

A treatment cycle can have different length. In some embodiments, a treatment cycle can be one week, 2 weeks, 3 weeks, 4 weeks, 5 weeks, 6 weeks, 7 weeks, 8 weeks, 3 months, 4 months, 5 months, 6 months, 7 months, 8 months, 9 months, 10 months, 11 months, or 12 months. In some embodiments, a treatment cycle is 3 weeks. In some embodiments, a treatment cycle is 4 weeks. A treatment cycle can have intermittent schedule. In some embodiments, a 2-week treatment cycle can have 5-day dosing followed by 9-day rest. In some embodiments, a 2-week treatment cycle can have 6-day dosing followed by 8-day rest. In some embodiments, a 2-week treatment cycle can have 7-day dosing followed by 7-day rest. In some embodiments, a 2-week treatment cycle can have 8-day dosing followed by 6-day rest. In some embodiments, a 2-week treatment cycle can have 9-day dosing followed by 5-day rest. In some embodiments, a 3-week treatment cycle can have 7-day dosing, followed by 14-day rest. In some embodiments, a 3-week treatment cycles can have 14-day dosing followed by 7-day rest. In some embodiment, a 4 week treatment cycle can have 7 day dosing, followed by 21-day rest. In some embodiment, a 4 week treatment cycle can have 14 day dosing, followed by 14-day rest.In some embodiment, a 4 week treatment cycle can have 21 day dosing, followed by 7-day rest. In some embodiment, a 4 week treatment cycle can have dosing on days 1-7 and 15-21, and rest on days 8-14 and 22-28.

In some embodiments, the FTI can be administered for at least one treatment cycle. In some embodiments, the FTI can be administered for at least two, at least three, at least four, at least five, at least six, at least seven, at least eight, at least nine, at least ten, at least eleven, or at least twelve treatment cycles. In some embodiments, the FTI can be administered for at least two treatment cycles. In some embodiments, the FTI can be administered for at least three treatment cycles. In some embodiments, the FTI can be administered for at least six treatment cycles. In some embodiments, the FTI can be administered for at least nine treatment cycles. In some embodiments, the FTI can be administered for at least twelve treatment cycles. In some embodiments, the FTI is tipifarnib.

In some embodiments, the FTI is administered for up to two weeks. In some embodiments, the FTI is administered for up to three weeks, up to one month, up to two months, up to three months, up to four months, up to five months, up to six months, up to seven months, up to eight months, up to nine months, up to ten months, up to eleven months, or up to twlve months. In some embodiments, the FTI is administered for up to one month. In some embodiments, the FTI is administered for up to three months. In some embodiments, the FTI is administered for up to six months. In some embodiments, the FTI is administered for up to nine months. In some embodiments, the FTI is administered for up to twelve months. In some embodiments, the FTI treatment therapy can be maintained for at least 6 months beyond the start of the response.

In some embodiments, the FTI is administered daily for 1 (days 1-7) of out of 3 weeks in repeated 3 week cycles. In some embodiments, the FTI is administered daily for 2 (days 1-14) of out of 3 weeks in repeated 3 week cycles. In some embodiments, the FTI is administered at a dose of 300 mg b.i.d. orally for 1(days 1-7) of out of 3 weeks in repeated 3 week cycles. In some embodiments, the FTI is administered at a dose of 300 mg b.i.d. orally for 2 (days 1-14) of out of 3 weeks in repeated 3 week cycles. In some embodiments, the FTI is administered daily for 3 of out of 4 weeks in repeated 4 week cycles. In some embodiments, the FTI is administered daily in alternate weeks (one week on, one week off) in repeated 4 week cycles. In some embodiments, the FTI is administered at a dose of 300 mg b.i.d. orally for 3 of out of 4 weeks in repeated 4 week cycles. In some embodiments, the FTI is administered at a dose of 600 mg b.i.d. orally for 3 of out of 4 weeks in repeated 4 week cycles. In some embodiments, the FTI is administered at a dose of 900 mg b.i.d. orally in alternate weeks (one week on, one week off) in repeated 4 week cycles. In some embodiments, the FTI is administered at a dose of 1200 mg b.i.d. orally in alternate weeks (days 1-7 and 15-21 of repeated 28-day cycles). In some embodiments, the FTI is administered at a dose of 1200 mg b.i.d. orally for days 1-5 and 15-19 out of repeated 28-day cycles.

In some embodiments, a 300 mg bid tipifarnib alternate week regimen can be used adopted. Under the regimen, patients receive a starting dose of 300 mg, po, bid on days 1-7 and 15-21 of 28-day treatment cycles. In the absence of unmanageable toxicities, subjects can continue to receive the tipifarnib treatment for up to 12 months. The dose can also be increased to 1200 mg bid if the subject is tolerating the treatment well. Stepwise 300 mg dose reductions to control treatment-related, treatment-emergent toxicities can also be included.

In some embodiments, a 600 mg bid tipifarnib alternate week regimen can be used adopted. Under the regimen, patients receive a starting dose of 600 mg, po, bid on days 1-7 and 15-21 of 28-day treatment cycles. In the absence of unmanageable toxicities, subjects can continue to receive the tipifarnib treatment for up to 12 months. The dose can also be increased to 1200 mg bid if the subject is tolerating the treatment well. Stepwise 300 mg dose reductions to control treatment-related, treatment-emergent toxicities can also be included.

In some embodiments, a 900 mg bid tipifarnib alternate week regimen can be used adopted. Under the regimen, patients receive a starting dose of 900 mg, po, bid on days 1-7 and 15-21 of 28-day treatment cycles. In the absence of unmanageable toxicities, subjects can continue to receive the tipifarnib treatment for up to 12 months. The dose can also be increased to 1200 mg bid if the subject is tolerating the treatment well. Stepwise 300 mg dose reductions to control treatment-related, treatment-emergent toxicities can also be included.

In some other embodiments, tipifarnib is given orally at a dose of 300 mg bid daily for 21 days, followed by 1 week of rest, in 28-day treatment cycles (21-day schedule; Cheng D T, et al., J Mol Diagn. (2015) 17(3):251-64). In some embodiments, a 5-day dosing ranging from 25 to 1300 mg bid followed by 9-day rest is adopted (5-day schedule; Zujewski J., J Clin Oncol., (2000) February; 18(4):927-41). In some embodiments, a 7-day bid dosing followed by 7-day rest is adopted (7-day schedule; Lara P N Jr., Anticancer Drugs., (2005) 16(3):317-21; Kirschbaum M H, Leukemia., (2011) October; 25(10):1543-7). In the 7-day schedule, the patients can receive a starting dose of 300 mg bid with 300 mg dose escalations to a maximum planned dose of 1800 mg bid. In the 7-day schedule study, patients can also receive tipifarnib bid on days 1-7 and days 15-21 of 28-day cycles at doses up to 1600 mg bid.

In some embodiments, the tipifarnib is administered at a dose of 300 mg twice daily on days 1-14 of 21-day cycles. In some embodiments, the tipifarnib is administered at a dose of 300 mg twice daily on days 1-14 of 21-day cycles and capecitabine is administered at a dose of 1,000 mg/m2 twice daily on days 1-14 of 21-day cycles.

In previous studies FTI were shown to inhibit the growth of mammalian tumors when administered as a twice daily dosing schedule. It was found that administration of an FTI in a single dose daily for one to five days produced a marked suppression of tumor growth lasting out to at least 21 days. In some embodiments, FTI is administered at a dosage range of 50-400 mg/kg. In some embodiments, FTI is administered at 200 mg/kg. Dosing regimen for specific FTIs are also well known in the art (e.g., U.S. Pat. No. 6,838,467, which is incorporated herein by reference in its entirety). For example, suitable dosages for the compounds Arglabin (WO98/28303), perrilyl alcohol (WO 99/45712), SCH-66336 (U.S. Pat. No. 5,874,442), L778123 (WO 00/01691), 2(S)-[2(S)-[2(R)-amino-3-mercapto]propylamino-3(S)-methyl]-pentyloxy-3-phenylpropionyl-methionine sulfone (WO94/10138), BMS 214662 (WO 97/30992), AZD3409; Pfizer compounds A and B (WO 00/12499 and WO 00/12498) are given in the aforementioned patent specifications which are incorporated herein by reference or are known to or can be readily determined by a person skilled in the art.

In relation to perrilyl alcohol, the medicament may be administered 1-4g per day per 150 lb human patient. In one embodiment, 1-2 g per day per 150 lb human patient. SCH-66336 typically may be administered in a unit dose of about 0.1 mg to 100 mg, more preferably from about 1 mg to 300 mg according to the particular application. Compounds L778123 and 1-(3-chlorophenyl)-4-[1-(4-cyanobenzyl)-5-imidazolylmethyl]-2-piperazinone may be administered to a human patient in an amount between about 0.1 mg/kg of body weight to about 20 mg/kg of body weight per day, preferably between 0.5 mg/kg of bodyweight to about 10 mg/kg of body weight per day.

Pfizer compounds A and B may be administered in dosages ranging from about 1.0 mg up to about 500 mg per day, preferably from about 1 to about 100 mg per day in single or divided (i.e. multiple) doses. Therapeutic compounds will ordinarily be administered in daily dosages ranging from about 0.01 to about 10 mg per kg body weight per day, in single or divided doses. BMS 214662 may be administered in a dosage range of about 0.05 to 200 mg/kg/day, preferably less than 100 mg/kg/day in a single dose or in 2 to 4 divided doses.

It is understood that modifications which do not substantially affect the activity of the various embodiments of this invention are also provided within the definition of the invention provided herein. Accordingly, the following examples are intended to illustrate but not limit the present invention. All of the references cited to herein are incorporated by reference in their entireties.

### 5. EXAMPLES

Throughout this application various publications have been referenced. The disclosures of these publications in their entireties, including GenBank and GI number publications, are hereby incorporated by reference in this application in order to more fully describe the state of the art to which this disclosure pertains. Although the invention has been described with reference to the examples provided above, it should be understood that various modifications can be made without departing from the scope of the invention.

### Example 1: Crosstalk between the IGF1 and CXCL12 Pathways Defines Objective Responses to the Farnesyl Transferase Inhibitor Tipifarnib in AML and PTCL Patients

Gene expression profile (GEP) data generated using RNA-Seq and the Affymetrix U133A gene-chip of tumor samples from 71 patients enrolled in tipifarnib trials (CTEP-20, KO-TIP-002, KO-TIP-004) were analyzed with respect to study outcomes and complemented with analyses of mRNA expression in data sets from the cBioportal for Cancer Genomics. Clinical trial information: NCT00027872, NCT02464228, NCT02807272.

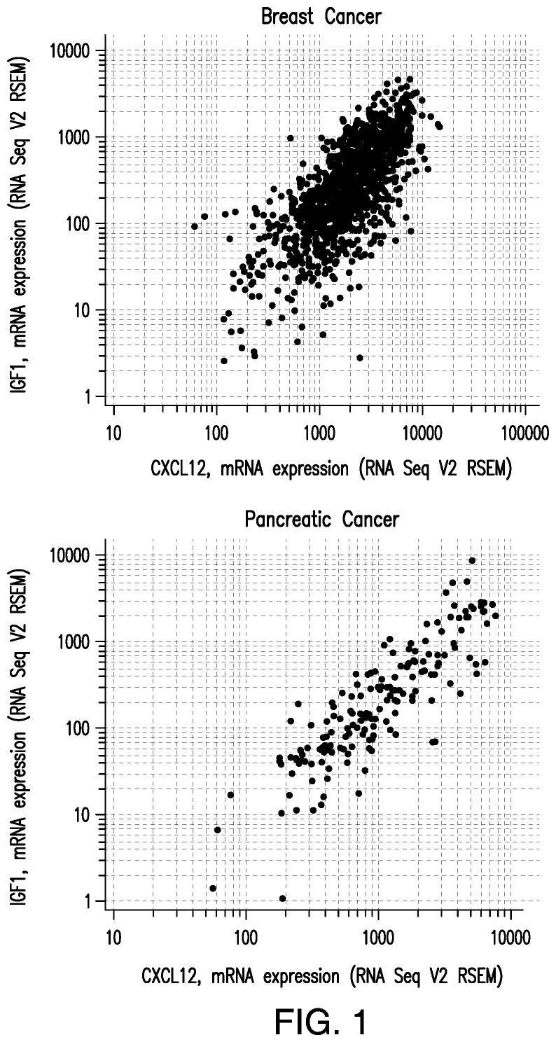

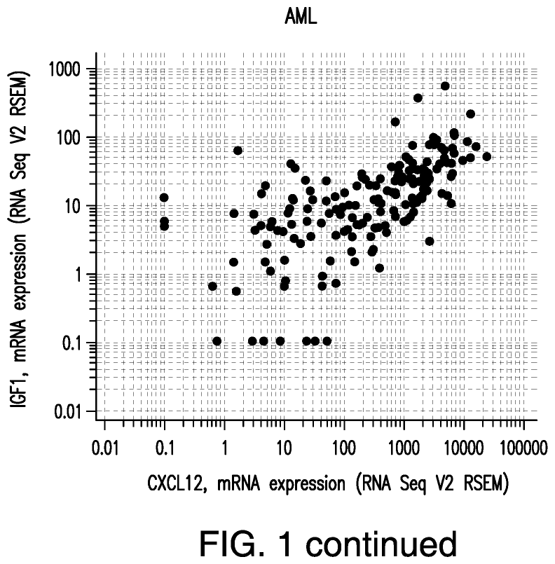

The pathology of tumor CXCL12 overexpression was investigated by examining the gene expression profiles from 8,401 cancer patients in 25 studies (available at cBioportal (TCGA, Provisional). There existed a highly significant correlation in the expression of the IGF1 and CXCL12 genes in 19 of those studies. These 25 studies are detailed below in Table 1. Highly significant correlations between the expression of the IGF1 and CXCL12 genes in breast cancer, pancreatic cancer and acute myeloid leukemia (AML) are also graphically shown in FIG. 1.

TABLE 1
Correlation of the Expression of IGF1 and CXCL12

| | CXCL12 vs IGF1 | |
|---|---|---|
| INDICATION | correlation (Spearman's) | N |
| Pancreatic cancer | 0.873 | 179 |
| Bladder cancer | 0.732 | 408 |
| Breast cancer | 0.719 | 1100 |
| Gastric cancer | 0.702 | 415 |
| Acute Myeloid leukemia | 0.698 | 173 |
| Colorectal cancer | 0.693 | 383 |
| Head and neck Cancer | 0.685 | 522 |
| Mesothelioma | 0.680 | 87 |
| Uveal melanoma | 0.663 | 80 |
| Glioblastoma | 0.613 | 166 |
| Adrenocortical carcinoma | 0.596 | 75 |
| Esophageal cancer | 0.596 | 186 |
| Melanoma | 0.588 | 498 |
| Lung adenocarcinoma | 0.565 | 517 |
| Prostate cancer | 0.558 | 472 |
| (CXCL12 vs IGF2) | | |
| Lung squamous carcinoma | 0.557 | 501 |
| Ovarian cancer | 0.536 | 307 |
| Hepatocellular carcinoma | 0.448 | 377 |
| (CXCL12 vs HGF) | | |
| Sarcoma | 0.391 | 498 |
| Prostate cancer | 0.391 | 259 |
| Diffuse B cell lymphoma | 0.351 | 48 |
| Kidney cancer | 0.191 | 534 |
| Pediatric ALL | 0.175 | 203 |
| Hepatocellular carcinoma | 0.051 | 377 |
| Cholangio carcinoma | 0.042 | 36 |

Of interest, the highest IGF1/CXCL12 correlations were observed in indications, including AML (ρ=0.698, p<0.0001, N=173), in which single agent activity of tipifarnib has been previously reported. These cancers with known monotherapy responses to tipifarnib are bladder cancer, breast cancer and AML (Table 1).

Data from a study investigating tipifarnib monotherapy in elderly patients with previously untreated AML (CTEP-20) was examined in detail to determine the effect of the expression of CXCL12 and IGF1 on the response of the patients to tipifarnib.

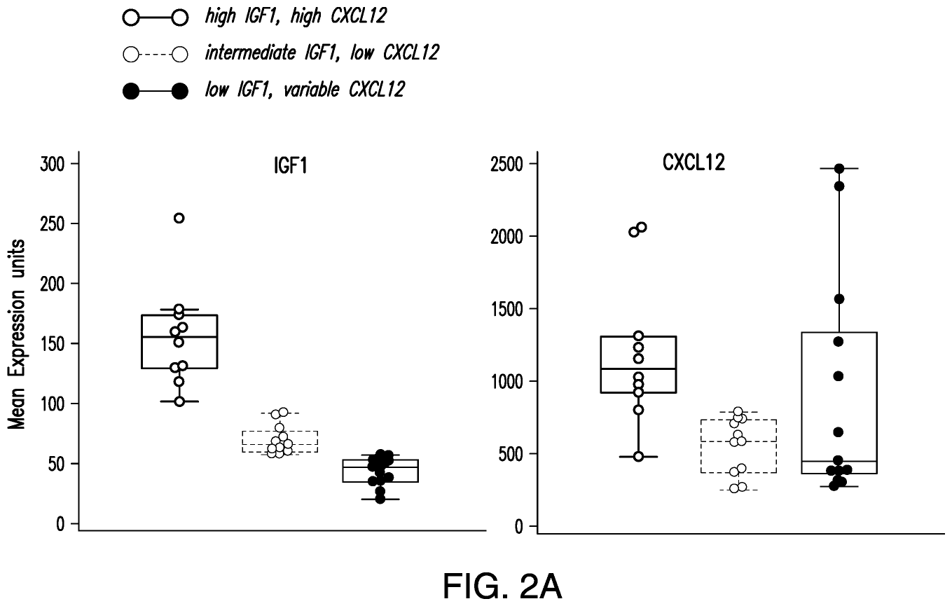

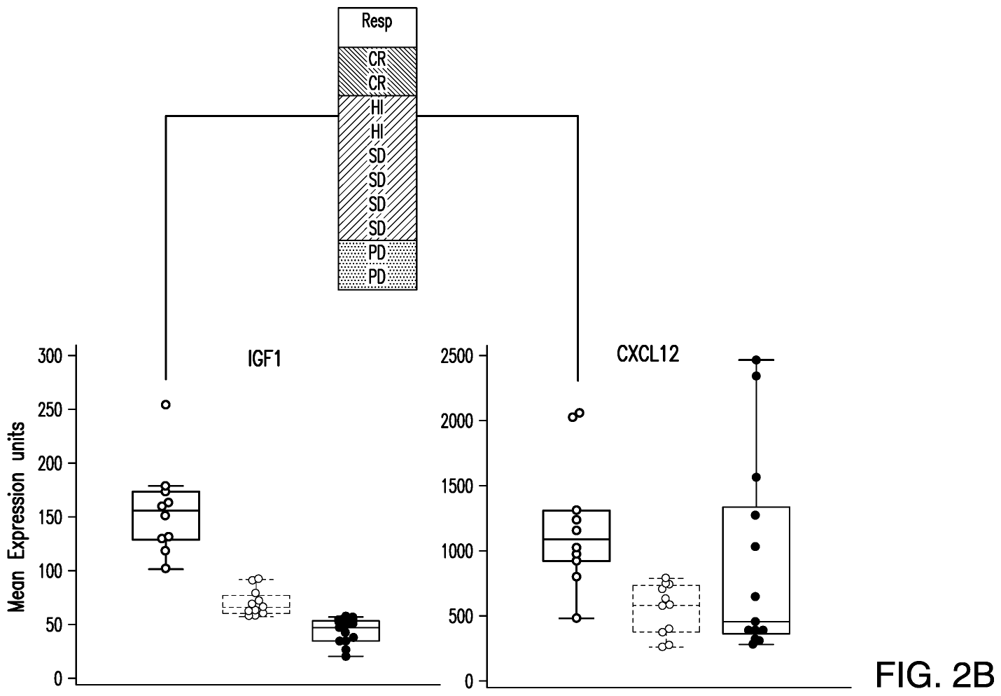

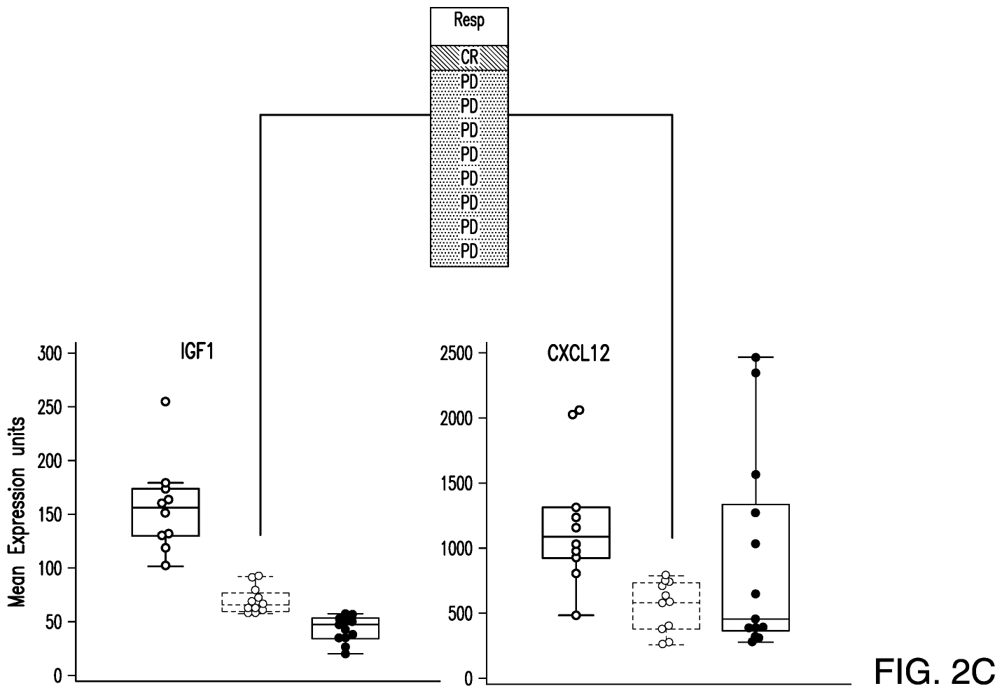

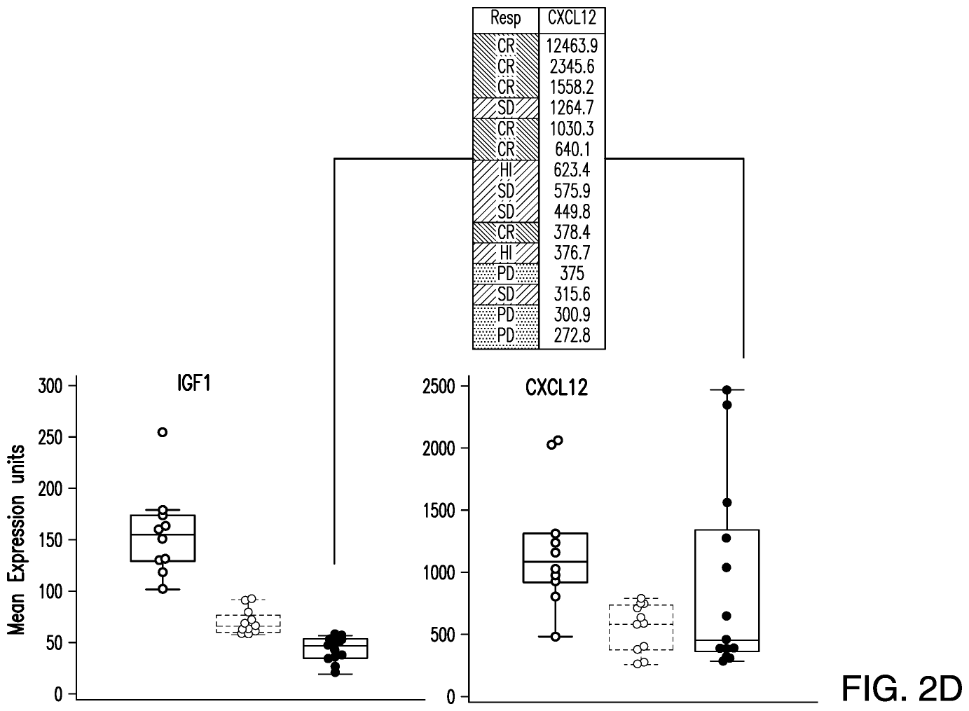

The effect of IGF1/CXCL12 co-expression on patient outcome in tipifarnib was examined. In previously untreated AML, 3 subsets of patients were identified with respect to IGF1/CXCL12 expression (FIG. 2A). These subsets were 1) high IGF1, high CXCL12 (left boxplots), 2) intermediate IGF1, low CXCL12 (middle boxplots), and 3) low IGF1, variable CXCL12, (right boxplots).

In the presence of high levels of IGF1, high levels of CXCL12 translated predominantly to an outcome of hematological improvement (HI) or stable disease (SD) with 2 patients having complete response (CR) as an outcome out of 10 subjects (FIG. 2B). When CXCL12 was at low levels and IGF1 was at intermediate levels, low CXCL12 levels translates to disease progression with 1 CR and 8 with progressive disease (PD) as outcome (FIG. 2C). When the IGF1 levels were low, CXCL12 had variable levels and six patients out of 15 experienced CR. CRs to tipifarnib were significantly associated with CXCL12 levels (p=0.013) (FIG. 2D). These data demonstrate that CXCL12 pathway activation determines objective responses with tipifarnib while high/intermediate IGF1 levels mediate drug resistance. Receiver-Operator-Characterisitics (ROC) curves for prediction of a CR in these patients confirmed that higher CXCL12 and lower IGF1 levels were predictive of complete responses in the AML patient population (FIG. 2E).

Four objective responses and 1 tumor lysis syndrome were reported in 17 tipifarnib-treated CMML subjects with available pre-treatment BM gene expression data. CXCL12 expression (p=0.07) and the CXCL12/CXCR4 ratio (p=0.03) were predictive of those events while IGF1 expression levels were barely detectable in these patients.

The effect of CXCL12 and IGF1 was also investigated in 13 nodal PTCL patients with pre-treatment GEP data who received tipifarnib monotherapy. High CXCL12 was predictive of 2 partial responses (PR) (p=0.009) despite the fact that those patients did not express low levels of IGF1. Further investigation revealed that the tumors of PTCL patients experiencing PR with tipifarnib expressed high levels of IGFBP7, an inhibitor of the IGF1 receptor (IGF1R) (Table 2). Adjusting the IGFBP7 expression to the level of IGF1R expression also identified those patients as responders to therapy.

TABLE 2
High CXCL12 and High IGFBP7 Identify Objective Responses in PTCL

| Group 1 | | | | | | | |
|---|---|---|---|---|---|---|---|
| Patient | | 2002 | 2004 | 3002 | 2001 | 4001 | 4004 |
| CXCL12 | | 469 | 759 | 613 | 2659 | 1160 | 3265 |
| IGF1 | | 295 | 137 | 174 | 382 | 13 | 207 |
| IGFBP7 | | 2906 | 4357 | 2444 | 3091 | 1777 | 10623 |
| Response | | PD | PD | PD | SD | SD | PR |
| IGF1R | | 3607 | 697 | 1167 | 1011 | 1807 | 1142 |
| IGFBP7/IGF1R | | 0.81 | 6.25 | 2.09 | 1.76 | 1.71 | 9.3 |
| Group 2 | | | | | | | |
| Patient | 2006 | 4005 | 4007 | 4006 | 2005 | 6003 | 2007 |
| CXCL12 | 1570 | 325 | 834 | 1211 | 1081 | 3728 | 14076 |
| IGF1 | 322 | 92 | 203 | 94 | 71 | 258 | 1076 |
| IGFBP7 | 6079 | 1805 | 3046 | 2560 | 5223 | 3183 | 11541 |
| Response | PD | PD | PD | PD | PD | SD | PR |
| IGF1R | 391 | 976 | 1203 | 644 | 1242 | 2127 | 1738 |
| IGFBP7/IGF1R | 4.62 | 3.12 | 4.34 | 3.98 | 4.89 | 1.5 | 6.64 |

Whole Exome Sequencing of PTCL pre-treatment biopsies revealed polymorphisms in the IGFBP7 gene in non-responding patients: No subject with a best response of PR, 1 of 4 pts with stable disease and 6 of 10 patients with PD carried the IGFBP7 variant L11F (rs11573021) (16% PR/SD vs 60% PD, p=0.15)

### Example 2: Identification of Clinical and Molecular Biomarkers Associated with clinical benefit of Tipifarnib in Advanced Pancreatic Cancer

As seen in Table 1 and FIG. 1, pancreatic cancer patients demonstrate a high correlation between the CXCL12 and IGF1 genes, higher than other tumor types in which tipifarnib activity has been observed. Pancreatic tumors also express very high levels of IGFB7. IGFBP7 was found to be coexpressed with IGF1 (p=0.643, p<0.0001, N=179) and CXCL12 (p=0.617, p<0.0001, N=173) in these tumors.

In a randomized, double-blind, placebo-controlled study of gemcitabine+tipifarnib versus gemcitabine+placebo in patients with advanced pancreatic adenocarcinoma previously untreated with systemic therapy, tipifarnib (R115777) was administered at a dosage amount of 200 mg twice a day, orally, and continuously (Study INT-17). Gemcitabine was administered intravenously at a dosage amount of 1,000 mg/m(2) weekly x 7 for 8 weeks, then weekly x 3 every 4 weeks.

686 patients were enrolled. The median overall survival for the experimental arm was 193 v 182 days for the control arm (P=0.75). Neutropenia and thrombocytopenia grade > or =3 were observed in 40% and 15% in the experimental arm versus 30% and 12% in the control arm.

While the results of the study showed that the combination of gemcitabine and tipifarnib does not prolong overall survival in advanced pancreatic cancer compared with single-agent gemcitabine, various biomarkers were examined for their relationship to therapeutic activity of tipifarnib.

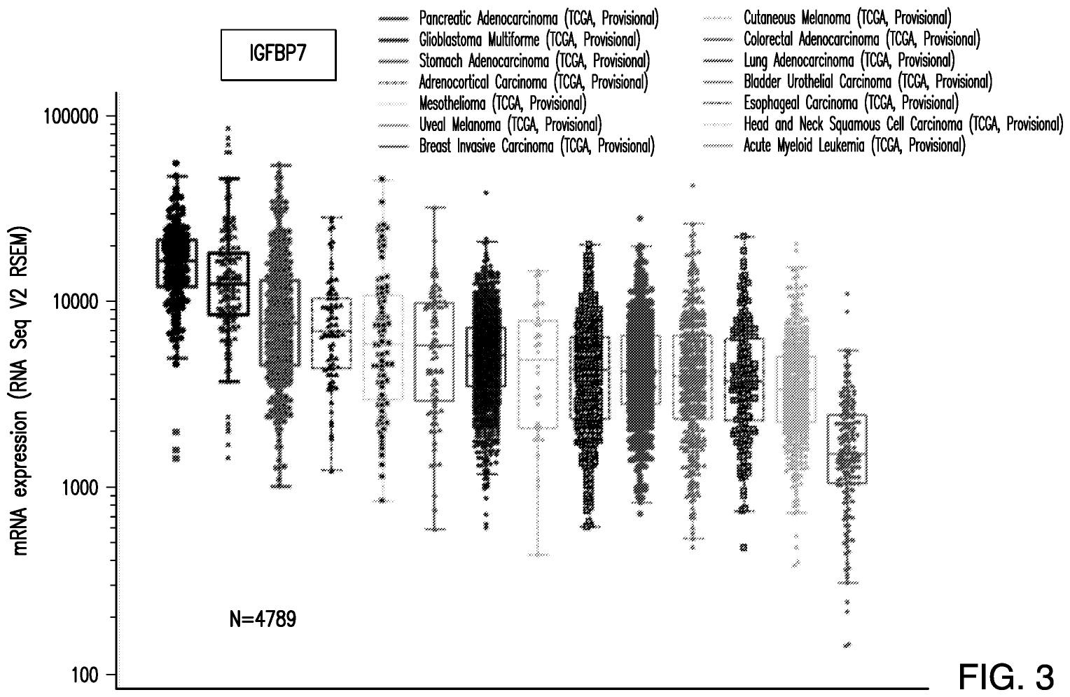

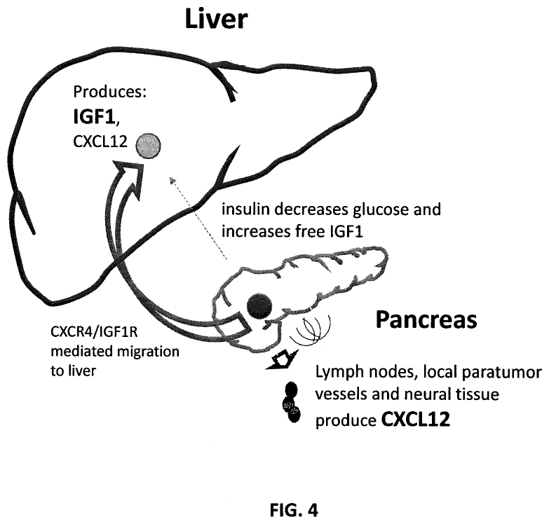

Pancreatic tumors may overexpress CXCL12 and IGF1 in different settings. Locally advanced disease, nodal disease would be expected to express high levels of CXCL12. In particular, pancreatic tumors that manifest with no abdominal pain are known to overexpress CXCL12. On the other hand, the liver is the main IGF1 producing organ, and pancreatic tumors in the liver are expected to express high levels of IGF1 and, by correlation, IGFBP7 and CXCL12 (FIG. 3).

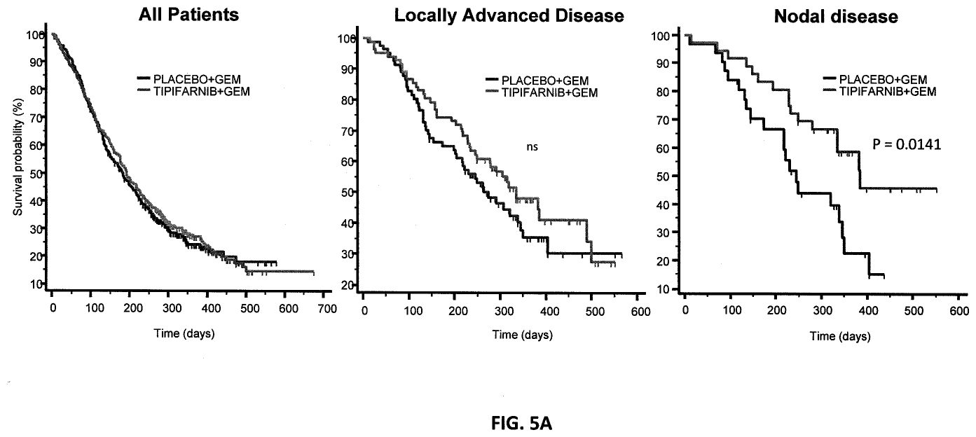

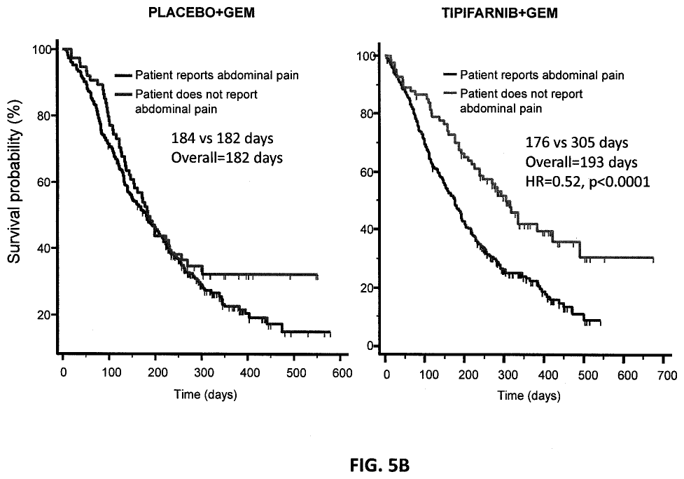

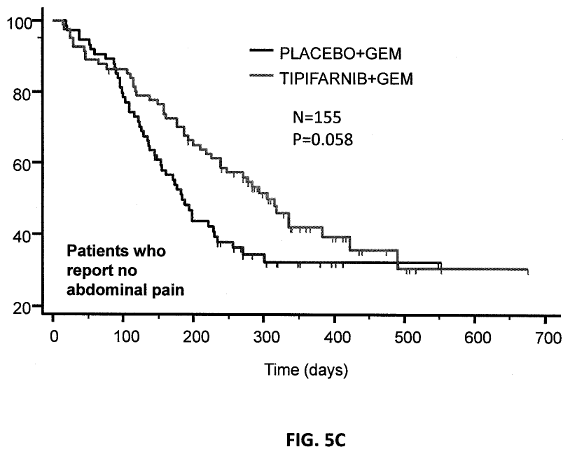

When all patients in this study were investigated as 2 overall treatment groups, no therapeutic benefit of tipifarnib was seen (FIG. 5A, left panel). A non-significant trend was observed in patients with locally advanced disease (LAD) (FIG. 5A, middle panel). Patients with tumors that spreaded to local lymph nodes (nodal disease) had better survival when treated with tipifarnib (FIG. 5A, right panel).

Abdominal pain is a hallmark clinical sign of pancreatic cancer. A subset of pancreatic cancer patients do not experience abdominal pain due to migration of peripheral nervous system Schwann cells to the tumor site by a mechanism that is dependent on CXCL12 overexpression. In study INT-11, the outcome of treatment with gemcitabine and placebo was not significantly different in patients who reported abdominal pain at study entry from those who did not (FIG. 5B, left). Patients who did not report abdominal pain had a significantly better survival from those who did not when treated with gemcitabine and tipifarnib (FIG. 5B, middle). A trend for better survival with gemcitabine and tipifarnib was observed when both treatment groups were compared in the subset of patients with no abdominal pain (FIG. 5c).

Taken together, these data indicate that clinical settings associated with high CXCL12 expression in pancreatic cancer such as local disease or absence of abdominal pain contribute to identify patient who may benefit from tipifarnib therapy.

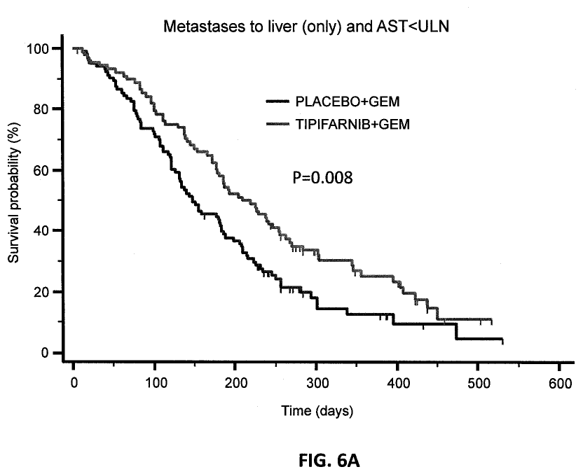

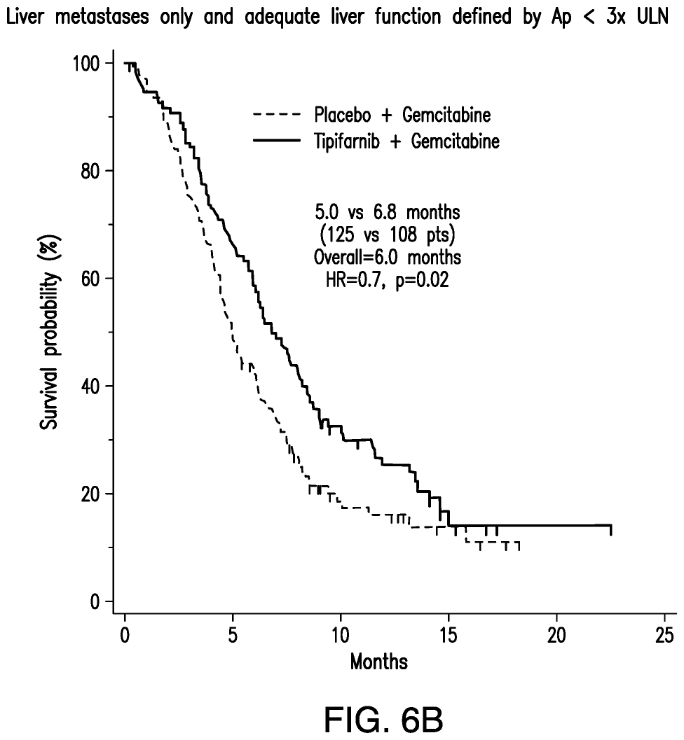

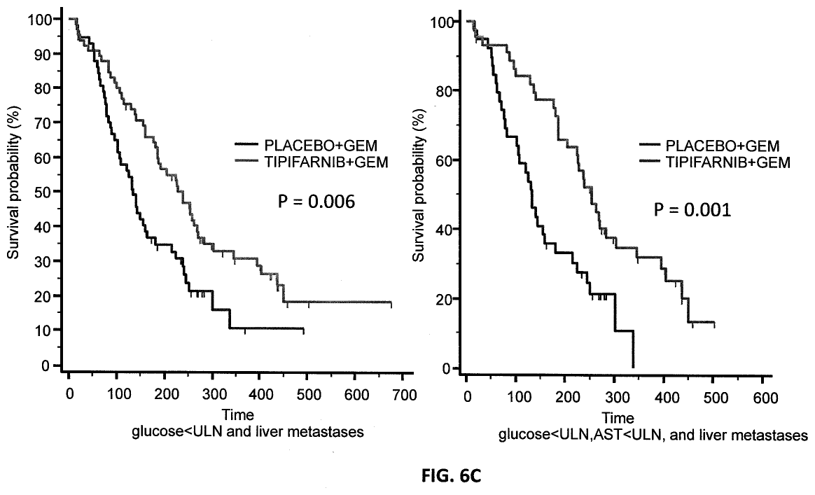

CXCR4 has been shown to functionally interact with the IGF1 receptor. IGF1 can induce the migration of cell towards IGF1 producing sites. The liver produces large amounts of IGF1. Patients with pancreatic tumors that have metastasized to the liver but not in other organs and who still have acceptable liver function manifested by aspartate transaminase within its limit of normal experienced a significant survival benefit when treated with tipifarnib and gemcitabine vs placebo and gemcitabine (FIG. 6A). No survival benefit with tipifarnib was observed when tumors metastasized to the lung or other organs. Survival benefit was also observed when tumors metastasized to the liver (only) and acceptable liver function was verified using bilirubin, alanine transaminase or alkaline phosphatase within limits of normal. Survival benefit with tipifarnib was also observed in patients previously treated with folfirinox (FIG. 6B).

IGF1 is controlled in the instertitium by small IGFBP proteins (IGFBP1 and 2) that are themselves controlled by insulin. Insulin maintains glucose levels low as part of its primary functions. Patients with pancreatic tumors that have metastasized to the liver (only) and who were not hyperglycemic at study entry experienced a survival benefit then treated with tipifarnib (FIG. 6C, left). Patients with pancreatic tumors that have metastasized to the liver (only) and who had normal liver function and were not hyperglycemic at study entry experienced improved survival benefit then treated with tipifarnib (FIG. 6C, right).

Taken together, these data indicate that clinical settings associated with IGF1 expression, and its correlate IGFBP7 in pancreatic cancer, such as presence of liver metastases, normal liver function and absence of hyperglycema contribute to identify patient who may benefit from tipifarnib therapy.

### Example 3: Low KRAS Mutation Allele Frequencies Identified Pancreatic Cancer Patients Likely to Receive Clinical Benefit from Tipifarnib

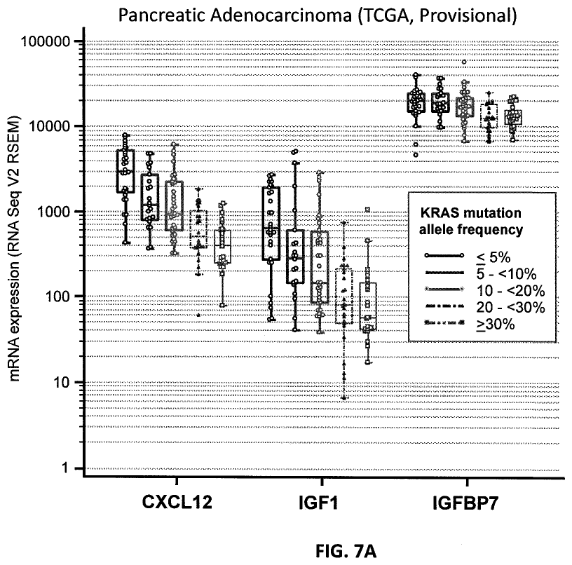

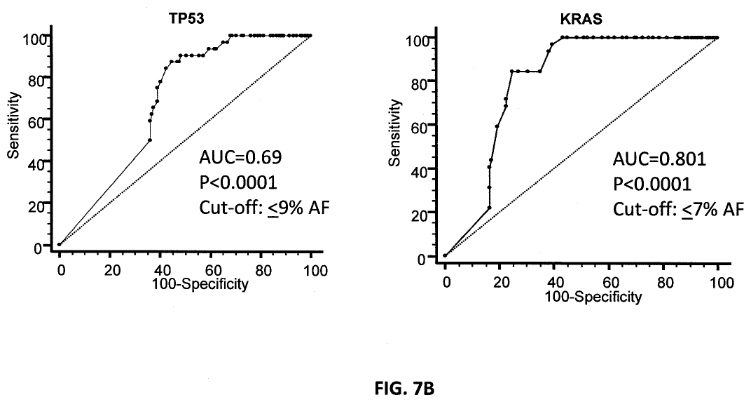

Low KRAS mutation allele frequency in pancreatic tumor samples was associated with high CXCL12 and high IGF1 gene expression. IGFBP7, however, was overall highly expressed in pancreatic cancer but trended to increase with lower KRAS mutation allele frequency (FIG. 7A). Therefore, low KRAS mutation allele frequency could identify pancreatic cancer patients with high CXCL12 who would susceptible to receive clinical benefit from tipifarnib. Likewise, low KRAS mutation allele frequency could identify pancreatic cancer patients with the highest IGFBP7 expression that could block any potential resistance to tipifarnib mediated by IGF1 (FIG. 7A).

Expression of CXCL12 was also elevated in tumors with wild type TP53 status or low allele frequency of TP53 mutation (FIG. 7B, left panel). The optimal cut-off of TP53 mutation allele frequency for CXCL12 overexpression was <9%. The optimal cut-off of KRAS mutation allele frequency for CXCL12 overexpression is <7% (FIG. 7B, right panel).

### Example 4: CXCL12 Expression and Other Molecular Markers Associated with Clinical Benefit of Tipifarnib Treatment in Breast Cancer Patients

Biomarkers in breast cancer were also examined for their relationship to CXCL12/IGF1 expression and to the activity of tipifarnib when administered to patients. A phase II study was performed with 76 patients treated with tipifarnib (Study GBR-1). Patients were tested for estrogen-receptor positivity (ER+) or negativity (ER−), progesterone receptor positivity (PGR+) or negativity (PGR−), and the presence or absence of TP53 mutation. Overall response rate in the study was 12%. Biomarker data was available for 41 patients.

Data in the TCGA database indicated that CXCL12 expression correlated inversely with estrogen receptor (ESR1) expression and directly with progesterone receptor expression (PGR) (Table 3). There was no significant relationship between IGF1 expression and PGR expression.

Data in the TCGA database indicated that CXCL12 expression correlated inversely TP53 mutation allele frequency.

Taken together these data indicate that PGR positivity and absence of TP53 mutation enriches for high CXCL12 expression and susceptibility to response to tipifarnib. PGR positivity and ER negativity also enriches for CXCL12 expression.

TABLE 3
CXCL12, IGF1 expression in relation to ESR1, PGR expression
and TP53 mutation allele frequency in breast cancer

| | | CXCL12 | IGF1 |
|---|---|---|---|
| CXCL12 | Correlation coefficient | | 0.615 |
| | Significance Level P | | <0.0001 |
| | n | | 1100 |
| IGF1 | Correlation coefficient | 0.615 | |
| | Significance Level P | <0.0001 | |
| | n | 1100 | |
| ESR1 | Correlation coefficient | −0.157 | −0.125 |
| | Significance Level P | <0.0001 | <0.0001 |
| | n | 1100 | 1100 |
| PGR | Correlation coefficient | 0.112 | 0.034 |
| | Significance Level P | 0.0002 | 0.2577 |
| | n | 1100 | 1100 |
| TP53AF | Correlation coefficient | −0.206 | −0.191 |
| | Significance Level P | <0.0001 | <0.0001 |
| | n | 1100 | 1100 |

Seven patients had breast cancer tumors that were PGR(+) and wild type TP53

(TP53(−)). Four of those patients experienced objective responses to tipifarnib (57%).

TABLE 4
PGR (+) TP53 (−) patient population and response to tipifarnib

| ER | TP53 | PGR | RESPONSE |
|---|---|---|---|
| POSITIVE | NEGATIVE | POSITIVE | NO RESPONSE |
| POSITIVE | NEGATIVE | POSITIVE | RESPONSE |
| POSITIVE | NEGATIVE | POSITIVE | NO RESPONSE |
| NEGATIVE | NEGATIVE | POSITIVE | NO RESPONSE |
| NEGATIVE | NEGATIVE | POSITIVE | RESPONSE |
| POSITIVE | NEGATIVE | POSITIVE | RESPONSE |

Four patients had breast cancer tumors that were PGR(+) and ER(−). Three of those patients experienced objective responses to tipifarnib (75%).

TABLE 5
PGR(+) ER(−) patient population and response to tipifarnib

| ER | PGR | RESPONSE |
|---|---|---|
| NEGATIVE | POSITIVE | NO RESPONSE |
| NEGATIVE | POSITIVE | RESPONSE |
| NEGATIVE | POSITIVE | RESPONSE |

### Example 5: Tipifarnib-Containing Regimens Inhibit Tumor Growth of KRAS Wild-Type, CXCL12-Expressing Patient-Derived Xenograft Models of Pancreatic Adenocarcinoma

Female BALB/c nude or Nu/nu mice (6-8 weeks) were inoculated subcutaneously on the right flank with primary human tumor model fragment (2-3 mm in diameter) for tumor development. When average tumor size reached about 250-350 mm³, mice were randomly grouped into dosing groups. Animals were dosed with tipifarnib vehicle (20% w/v hydroxypropyl-β-cyclodextrin), tipifarnib at a dose of 80 mg/kg BID PO or a combination of tipifarnib (80 mg/kg BID PO), celecoxib (5 mg/kg QD PO) and atorvastatin (2 mg/kg QD PO) for 3-4 weeks and tumor dimensions were measured twice per week.

The ability of tipifarnib to inhibit tumor growth was determined using selected patient-derived xenograft (PDX) models of PDAC. The selected models expressed either wild-type or mutant KRAS and also expressed varying levels of human CXCL12, as indicted in Table 1.

TABLE 6
KRAS mutational status and CXCL12
mRNA expression in PDAC PDX models

| | | | CXCL12 mRNA |
|---|---|---|---|
| PDX Model | Cancer Type | KRAS genotype | (Log2 FKPM) |
| PA2409 | PDAC | Wild type | 5.44 |
| PA3546 | PDAC | Wild type | 7.11 |
| PA1280 | PDAC | Wild type | −2 |
| PA6259 | PDAC | Wild type | −2 |
| PA3006 | PDAC | G12D | 2.45 |
| PA6265 | PDAC | G12D | 4.31 |

* −2 Log2 units represent zero in this RNAseq dataset

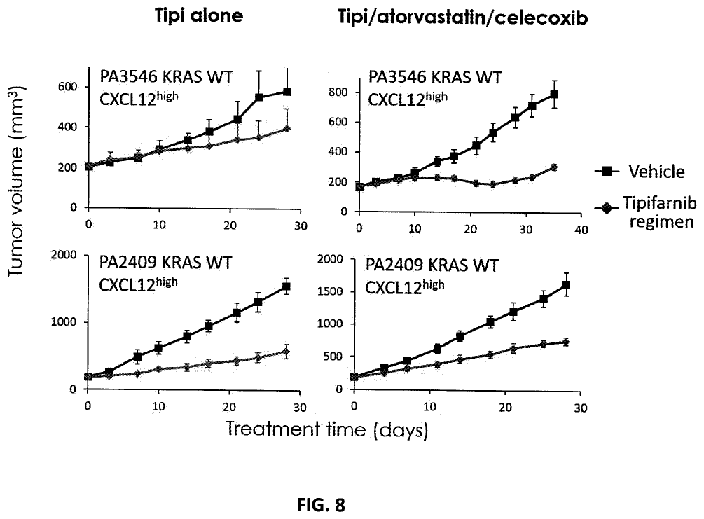

FIG. 8 shows the effectiveness of two different tipifarnib treatment regimens in reducing tumor volume in two different PDX models of KRAS wildtype-, CXCL12 mRNA expressing-PDAC. Mice were treated with either a combination consisting of tipifarnib, atorvastatin, and celecoxib or tipifarnib alone. As illustrated in FIG. 8, both tipifarnib treatment regimens reduced tumor volume of KRAS wild type-, CXCL12 expressing-PDAC.

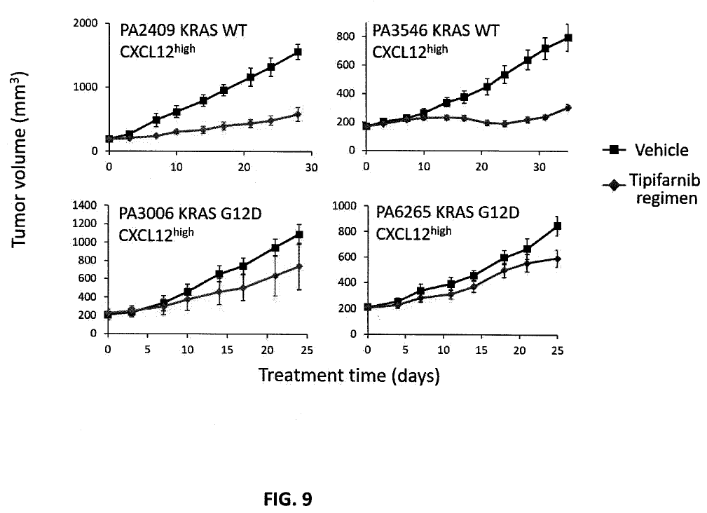

The effectiveness of tipifarnib treatment in reducing tumor volume was compared in four different PDX models of PDAC. The tumors of all four PDX models expressed high amounts of CXCL12 mRNA. FIG. 9 shows that tipifarnib administration was more effective at reducing tumor volume in tumors with wild type KRAS (PA2409 and PA3546) than tumors with a G12D KRAS mutation (PA3006 and PA6265).

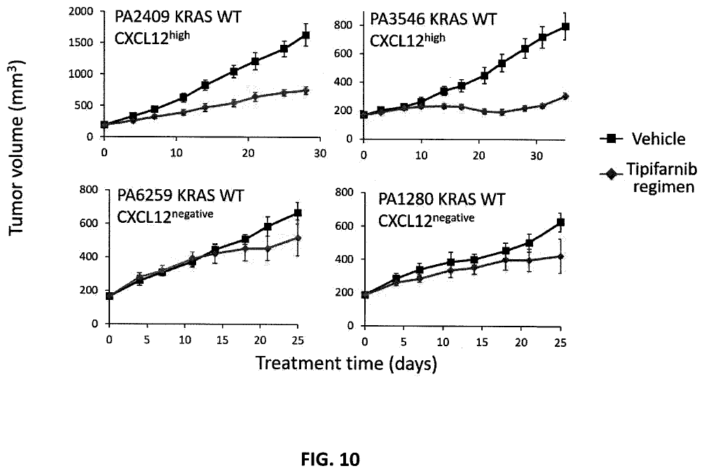

Similarly, high CXCL12 mRNA expression appears to be similarly important in predicting the effectiveness of tipifarnib as the KRAS VAF. The effectiveness of tipifarnib treatment in reducing tumor volume was compared in four different PDX models of KRAS-wildtype PDAC. FIG. 10 shows that tipifarnib administration was more effective at reducing tumor volume in tumors expressing high levels of CXCL12 mRNA (PA2409 and PA3546) than tumors expressing no CXCL12 (PA6259 and PA1280). Taken together, these data show that tipifarnib treatment reduced tumor volume in KRAS wild type tumors that express high amounts of CXCL12 in PDX models of PDAC.

### Example 6: A Randomized Phase 2 Study of Tipifarnib in Combination with Capecitabine vs. Capecitabine Alone in Subjects with Metastatic Adenocarcinoma of the Pancreas After Failure of Gemcitabine-Containing Chemotherapy

This clinical trial will evaluate the OS rate of subjects treated with tipifarnib in combination with capecitabine in subjects with histologically or cytologically confirmed PDCA that is metastatic and not amenable to local therapy with curative intent. Subjects must be refractory or have relapsed from no more than 1 prior gemcitabine-containing chemotherapy regimen.

To participate in this study, all subjects must have measurable disease that meets the criteria for selection as a target lesion according to Response Evaluation Criteria in Solid Tumors (RECIST) v1.1. The presence of at least one measurable target lesion per RECIST v1.1 must be confirmed by local radiology prior to enrollment. Subjects without at least one measurable target lesion confirmed by local radiology will not be enrolled into the study. Additionally, subjects must have at least 1 metastatic lesion in the liver.

Enrolled subjects must not have a known tumor missense KRAS mutation (defined as a missense KRAS Variant Allele Frequency, VAF, >5%) based on centralized testing. KRAS status may be assessed on tumor obtained at primary diagnosis or in recurrent or metastatic disease. If several samples are available, KRAS testing should be performed in the most recently obtained tumor sample.

Patients will be randomized 2:1 to the combination of tipifarnib and capecitabine or capecitabine alone. At least 35 subjects will be randomized (2:1) to one of two treatment groups:

The first treatment group will receive a treatment regimen comprising tipifarnib and capecitabine. Subjects in this group will receive oral capecitabine 1,000 mg/m2 twice daily plus oral tipifarnib 300 mg twice daily with food on days 1-14 of 21-day cycles. The second treatment group will receive a treatment regimen of only capecitabine. Subjects in this group will receive oral capecitabine 1,250 mg/m2 twice daily on days 1-14 of 21-day cycles.

Subjects in the combination arm who discontinue treatment with capecitabine for any reason may continue treatment with tipifarnib and vice versa. In the absence of unmanageable toxicities, subjects may continue to receive treatment until disease progression. If a complete response is observed, therapy will be maintained for at least 6 months beyond the start of response. In the combination arm, if capecitabine is discontinued for any reason, subjects may continue treatment with tipifarnib and vice versa.

One interim assessment for futility in the combination arm at 14 patients enrolled is planned.

Tumor assessment will be performed by the Investigator according to RECIST v1.1. Assessments will be performed at screening and approximately every 6 weeks for the first 12 months of a subject's participation; thereafter, tumor response assessment should occur approximately every 12 weeks until 2 years from the start of study treatment or disease progression. Radiological assessments will be discontinued at the time of tumor progression. If the subject initiates a new anticancer therapy without evidence of disease progression by RECIST v1.1, tumor scans should continue until there is evidence of disease progression unless withdrawal of subject's consent to study procedures.

Upon disease progression, all subjects will be followed approximately every 12 weeks for survival and the use of subsequent therapy until either death or End of Study (up to 2 years since the enrollment of the last study subject), whichever occurs first.

All subjects will be followed-up for safety through approximately 30 days after treatment discontinuation or until immediately before the administration of another anticancer treatment, whichever occurs first. Additional safety follow-up may be conducted if unresolved toxicity is present at the End of Treatment visit.

We claim:

1. A method of treating a KRAS wild type cancer in a subject, comprising administering a therapeutically effective amount of a farnesyltransferase inhibitor (FTI) to the subject.

2. The method of claim 1, wherein the subject has a greater C-X-C motif chemokine ligand 12 (CXCL12) expression than a reference level of CXCL12 expression.

3. The method of claim 1, wherein the cancer is a solid tumor.

4. The method of claim 3, wherein the solid tumor is pancreatic cancer.

5. The method of claim 4 wherein the cancer is pancreatic ductal adenocarcinoma (PDAC).

6. The method of any one of claims 3 to 5, wherein the tumor has a KRAS Variant Allele Frequency (VAF) of less than or equal to 5%.

7. The method of any one of claims 1 to 6, wherein the KRAS status is assessed at primary diagnosis or in recurrent or metastatic disease.

8. A method of treating a cancer in a subject comprising administering a therapeutically effective amount of a farnesyltransferase inhibitor (FTI) to said subject, wherein the subject has
(i) (a) a greater C-X-C motif chemokine ligand 12 (CXCL12) expression than a reference level of CXCL12 expression; or
(b) a CXCR4 expression greater than a reference level of CXCR4 expression; and
(ii) (a) a lower insulin-like growth factor 1 (IGF1) expression than a reference level of IGF1 expression; or
(b) a greater insulin-like growth factor binding protein 7 (IGFBP7) expression than a reference level of IGFBP7 expression.

9. The method of claim 8, wherein the subject has an IGF1 expression that is non-detectable.

10. The method of claim 8 or 9, wherein the subject further has (i) a lower insulin-like growth factor 2 (IGF2) expression than a reference level of IGF2 expression, or (ii) a greater insulin-like growth factor 2 receptor (IGF2R) expression than a reference level of IGF2R expression.

11. The method of claim 10, wherein the subject has an IGF2 expression that is non-detectable.

12. The method of any one of claims 8 to 11, wherein the subject does not have a loss of heterozygosity or loss of imprinting of the IGF2 gene.

13. The method of any one of claims 8 to 12, wherein the subject does not carry the IGFBP7 variant L11F (rs11573021).

14. The method of any one of claims 8 to 13, wherein the subject has a greater ratio of expression of CXCL12 to C-X-C chemokine receptor type 4 (CXCR4) than a reference ratio.

15. The method of any one of claims 8 to 14, wherein the subject further has an activating mutation in the CXCR4 gene.

16. The method of any one of claims 8 to 15, wherein the subject further has a greater ratio of CXCR4 to CXCR2 expression than a reference ratio.

17. The method of any one of claims 8 to 16, wherein the subject has a KRAS mutation allele frequency that is less than 15%, less than 12%, less than 10%, less than 8%, less than 7%, less than 6%, or less than 5%.

18. The method of claim 17, wherein the subject does not have an activating mutation in the KRAS gene.

19. The method of any one of claims 8 to 18, wherein the subject has a TP53 mutation allele frequency that is less than 15%, less than 12%, less than 10%, less than 8%, less than 7%, less than 6%, or less than 5%.

20. The method of claim 19, wherein the subject does not have a mutation in the TP53 gene.

21. The method of any one of claims 8 to 20, wherein the subject does not have an activating mutation in PI3K or AKT.

22. The method of any one of claims 8 to 21, further comprising measuring the expression level of CXCL12, IGF1, IGFBP7, IGF2, IGF2R, CXCR4, CXCR2, or any combination thereof, in a sample of the subject.

23. The method of claim 22, comprising measuring the protein level of CXCL12, IGF1, IGFBP7, IGF2, IGF2R, CXCR4, CXCR2, or any combination thereof, in the sample.

24. The method of claim 23, wherein the protein level is determined using a immunohistochemistry (IHC) approach, an immunoblotting assay, flow cytometry (FACS), or ELISA.

25. The method of claim 22, wherein measuring the mRNA level of CXCL12, IGF1, IGFBP7, IGF2, IGF2R, CXCR4, CXCR2, or any combination thereof, in the sample.

26. The method of claim 25, wherein the mRNA level is measured using qPCR, RT-PCR, RNA-seq, microarray analysis, SAGE, MassARRAY technique, or FISH.

27. The method of any one of claims 8 to 26, further comprising determining the mutation status of IGFBP7, KRAS, TP53, PI3K, AKT, CXCR4 or any combination thereof.

28. The method of any one of claims 22 to 27, wherein said sample is a tissue biopsy.

29. The method of any one of claims 22 to 27, wherein said sample is a tumor biopsy.

30. The method of any one of claims 22 to 27, wherein the sample is isolated cells.

31. The method of any one of claims 8 to 30, wherein cancer is a hematological cancer.

32. The method of claim 31, wherein the hematological cancer is a myeloid hematological cancer selected from the group consisting of acute myeloid leukemia (AML), myeloproliferative neoplasm (MPN), myelodysplastic syndrome (MDS), chronic myelomonocytic leukemia (CMML) and chronic myeloid leukemia (CML).

33. The method of claim 32, wherein the hematological cancer is AML.

34. The method of claim 31, wherein the hematological cancer is a lymphoid hematological cancer selected from the group consisting of natural killer cell lymphoma (NK lymphoma), natural killer cell leukemia (NK leukemia), cutaneous T-Cell lymphoma (CTCL), and peripheral T-cell lymphoma (PTCL).

35. The method of claim 34, wherein the hematological cancer is PTCL.

36. The method of any one of claims 1 to 30, wherein the cancer is a solid tumor selected from the group consisting of pancreatic cancer, bladder cancer, breast cancer, gastric cancer colorectal cancer, head and neck cancer, mesothelioma, uveal melanoma, glioblastoma, adrenocortical carcinoma, esophageal cancer, melanoma, lung adenocarcinoma, prostate cancer, lung squamous carcinoma, ovarian cancer, hepatocellular carcinoma, sarcoma, and prostate cancer.

37. The method of claim 36, wherein the solid tumor is pancreatic cancer, bladder cancer, breast cancer or gastric cancer.

38. The method of claim 36, wherein the solid tumor is breast cancer.

39. The method of claim 38, wherein the breast cancer is progesterone receptor (PR) positive.

40. The method of claim 38 or claim 39, wherein the breast cancer is estrogen receptor (ER) negative.

41. A method of treating a pancreatic cancer in a subject comprising administering a therapeutically effective amount of a FTI to said subject, wherein the subject has
(i) liver metastases; and
(ii) (1) an aspartate transaminase (AST) level, (2) an alanine transaminase level, (3) an alkaline phosphatase, and/or (4) a total bilirubin level that is no more than the normal upper limit.

42. A method of treating a pancreatic cancer in a subject comprising administering a therapeutically effective amount of a FTI to said subject, wherein the subject (i) has nodal metastasis, and (ii) does not have abdominal pain.

43. The method of claim 41 or claim 42, wherein the solid tumor is pancreatic ductal adenocarcinoma (PDAC).

44. The method of any one of claims 1 to 43, further comprising administering an inhibitor of IGF1R pathway to said subject.

45. The method of claim 44, wherein FTI is administered before, during, or after the administration of said inhibitor of IGF1R pathway.

46. The method of claim 44 or claim 45, wherein said inhibitor of IGF1R pathway is selected from the group consisting of an IGF1 inhibitor, an IGF1R inhibitor, a PI3K inhibitor, and an AKT inhibitor.

47. The method of claim 46, wherein the inhibitor of IGF1R pathway is an anti-IGF1 antibody.

48. The method of claim 46, wherein the inhibitor of IGF1R pathway is an IGF1R inhibitor selected from the group consisting of dalotuzumab, robatumumab, figitumumab, cixutumumab, ganitumab, AVE1642, OSI-906, NVP-AEW541 and NVP-ADW742.

49. The method of claim 46, wherein the inhibitor of IGF1R pathway is a PI3K inhibitor selected from the group consisting of SF1126, TGX-221, PIK-75, PI-103, SN36093, IC87114, AS-252424, AS-605240, NVP-BEZ235, GDC-0941, ZSTK474, LY294002 and wortmannin.

50. The method of claim 46, wherein the inhibitor of IGF1R pathway is an AKT inhibitor selected from the group consisting of perifosine, SR13668, A-443654, triciribine phosphate monohydrate, GSK690693, and deguelin.

51. The method of any one of claims 1 to 50, further comprising administering a radiation therapy.

52. The method of any one of claims 1 to 51, further comprising administering a therapeutically effective amount of an additional active agent.

53. The method of claim 52, wherein said additional active agent is selected from the group consisting of a DNA-hypomethylating agent, an alkylating agent, a topoisomerase inhibitor, a therapeutic antibody that specifically binds to a cancer antigen, a hematopoietic growth factor, a cytokine, an antibiotic, a cox-2 inhibitor, a CDK inhibitor, an immunomodulatory agent, an anti-thymocyte globulin, an immunosuppressive agent, and a corticosteroid or a pharmacological derivative thereof.

54. The method of claim 52, wherein said additional active agent is capecitabine.

55. The method of claim 54, wherein the capecitabine is administered at a dose of 1-1000 mg/m².

56. The method of claim 54 or 55, wherein the capecitabine is administered twice a day.

57. The method of any one of claims 54 to 56, wherein the capecitabine is administered on days 1-7 of 21-day cycles.

58. The method of any one of claims 54 to 56, wherein the capecitabine is administered on days 1-14 of 21-day cycles.

59. The method of claim 52, wherein said additional active agent is an anti-PD1 antibody, an anti-PDL1 antibody, or an anti-CTLA-4 antibody.

60. A method of treating a cancer in a subject, comprising administering a therapeutically effective amount of a FTI and a therapeutically effective amount of (i) an inhibitor of IGF1R pathway or (ii) an CXCR4 antagonist to said subject.

61. The method of claim 60, wherein the cancer is a solid tumor.

62. The method of claim 61, wherein the solid tumor is selected from the group consisting of pancreatic cancer, bladder cancer, breast cancer, gastric cancer colorectal cancer, head and neck cancer, mesothelioma, uveal melanoma, glioblastoma, adrenocortical carcinoma, esophageal cancer, melanoma, lung adenocarcinoma, prostate cancer, lung squamous carcinoma, ovarian cancer, hepatocellular carcinoma, sarcoma, and prostate cancer.

63. The method of claim 62, wherein the solid tumor is pancreatic cancer, bladder cancer, breast cancer or gastric cancer.

64. The method of claim 63, wherein the solid tumor is pancreatic cancer.

65. The method of claim 64, wherein the pancreatic cancer is pancreatic ductal adenocarcinoma (PDAC).

66. The method of claim 63, wherein the solid tumor is breast cancer.

67. The method of claim 60, wherein the cancer is a hematological cancer.

68. The method of claim 67, wherein the hematological cancer is selected from the group consisting of acute myeloid leukemia (AML), myeloproliferative neoplasm (MPN), myelodysplastic syndrome (MDS), chronic myelomonocytic leukemia (CMML), chronic myeloid leukemia (CIVIL), natural killer cell lymphoma (NK lymphoma), natural killer cell leukemia (NK leukemia), cutaneous T-Cell lymphoma (CTCL), and peripheral T-cell lymphoma (PTCL).

69. The method of any one of claims 60 to 68, wherein the FTI is administered before, during, or after the administration of the inhibitor of IGF1R pathway or the CXCR4 antagonist.

70. The method of any one of claims 60 to 69, comprising administering an FTI with an CXCR4 antagonist that is selected from the group consisting of AMD-3100, BL-8040, chloroquine, and plerixafor.

71. The method of any one of claims 60 to 69, comprising administering an FTI with an inhibitor of IGF1R pathway that is selected from the group consisting of an IGF1 inhibitor, an IGF1R inhibitor, a PI3K inhibitor, and an AKT inhibitor.

72. The method of claim 70, wherein the inhibitor of IGF1R pathway is an anti-IGF1 antibody.

73. The method of claim 70, wherein the inhibitor of IGF1R pathway is an IGF1R inhibitor selected from the group consisting of dalotuzumab, robatumumab, figitumumab, cixutumumab, ganitumab, AVE1642, OSI-906, NVP-AEW541 and NVP-ADW742.

74. The method of claim 70, wherein the inhibitor of IGF1R pathway is a PI3K inhibitor selected from the group consisting of SF1126, TGX-221, PIK-75, PI-103, SN36093, IC87114, AS-252424, AS-605240, NVP-BEZ235, GDC-0941, ZSTK474, LY294002 and wortmannin.

75. The method of claim 70, wherein the inhibitor of IGF1R pathway is an AKT inhibitor selected from the group consisting of perifosine, SR13668, A-443654, triciribine phosphate monohydrate, GSK690693, and deguelin.

76. The method of any one of claims 1 to 75, wherein the FTI is selected from the group consisting of tipifarnib, lonafarnib, arglabin, perrilyl alcohol, L778123, L739749, FTI-277, L744832, CP-609,754, R208176, AZD3409, and BMS-214662.

77. The method of claim 76, wherein the FTI is lonafarnib.

78. The method of claim 76, wherein the FTI is BMS-214662.

79. The method of claim 76, wherein the FTI is tipifarnib.

80. The method of claim 79, wherein tipifarnib is administered at a dose of 0.05-500 mg/kg body weight.

81. The method of claim 79 or claim 80, wherein tipifarnib is administered twice a day.

82. The method of claim 81, wherein tipifarnib is administered at a dose of 100-1200 mg twice a day.

83. The method of claim 82, wherein the tipifarnib is administered at a dose of 100 mg, 200 mg, 300 mg, 400 mg, 600 mg, 900 mg or 1200 mg twice a day.

84. The method of any one of claims 79 to 83, wherein the tipifarnib is administered on days 1-7 and 15-21 of a 28-day treatment cycle.

85. The method of any one of claims 79 to 83, wherein the tipifarnib is administered on days 1-21 of a 28-day treatment cycle.

86. The method of any one of claims 79 to 83, wherein the tipifarnib is administered on days 1-7 of a 28-day treatment cycle.

87. The method of any one of claims 79 to 83, wherein the tipifarnib is administered on days 1-7 of 21-day cycles.

88. The method any one of claims 79 to 83, wherein the tipifarnib is administered on days 1-14 of 21-day cycles.

89. The method of any one of claims 79 to 83, wherein the tipifarnib is administered at a dose of 300 mg twice daily on days 1-14 of 21-day cycles and the capecitabine is administered at a dose of 1,000 mg/m2 twice daily on days 1-14 of 21-day cycles.

90. The method of any one of claims 84 to 89, wherein tipifarnib is administered for at least 2 cycles.

91. The method of claim 90, wherein tipifarnib is administered for at least 3 cycles, 6 cycles, 9 cycles, or 12 cycles.

92. The method of any one of claims 84 to 91, wherein therapy can be maintained for at least 6 months beyond the start of the response.
